(19) United States
(12) Patent Application Publication
(10) Pub. No.: US 2025/0059560 A1
(43) Pub. Date: Feb. 20, 2025

(54) METHODS AND SYSTEMS OF PARTICLE PRODUCTION

(71) Applicant: Sana Biotechnology, Inc., Seattle, WA (US)

(72) Inventors: Noelle COLANT, Somerville, MA (US); Kaely B. GALLAGHER, Somerville, MA (US); Yonatan Y. LIPSITZ, Seattle, WA (US); Jeffrey John UNDERHILL, Boston, MA (US)

(73) Assignee: Sana Biotechnology, Inc., Seattle, WA (US)

(21) Appl. No.: 18/719,792

(22) PCT Filed: Dec. 15, 2022
(86) PCT No.: PCT/US2022/081722

§ 371 Date: Jun. 13, 2024

Related U.S. Application Data
(60) Provisional application No. 63/290,567, filed on Dec. 16, 2021.

(51) Int. Cl. C12N 15/86; C12N 7/00

(52) U.S. Cl. CPC C12N 15/86; C12N 7/00; C12N 2740/15023; C12N 2740/15052; C12N 2760/18045; C12N 2760/18222

(57) ABSTRACT

Provided herein are methods of producing lipid membrane bound particles, such as lentiviral vectors, that contain one or more Paramyxovirus envelope proteins in the lipid bilayer. Also provided are lipid membrane bound particles containing one or more Paramyxovirus envelope proteins in the lipid bilayer. In some embodiments, the one or more Paramyxovirus envelope protein is a Nipah virus (NiV) protein G or F or a biologically active portion or retargeted fusion thereof. In some embodiments, the lipid membrane bound particle is a lentiviral vector. Also provided are related compositions, kits and systems in connection with the provided methods.

### CROSS-REFERENCE TO RELATED APPLICATIONS

This application claims priority to U.S. provisional application 63/290,567 entitled “Methods and Systems of Particle Production,” filed Dec. 16, 2021, the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 18615_2004340.xml created Dec. 13, 2022 which is 84,392 bytes in size. The information in the electronic format of the Sequence Listing is incorporated by reference in its entirety.

### FIELD

The present disclosure provides methods of producing lipid membrane bound particles, such as lentiviral vectors, that contain one or more Paramyxovirus envelope proteins in the lipid bilayer. Also provided are lipid membrane bound particles containing one or more Paramyxovirus envelope proteins in the lipid bilayer. In some embodiments, the one or more Paramyxovirus envelope protein is a Nipah virus (NiV) protein G or F or a biologically active portion or retargeted fusion thereof. In some embodiments, the lipid membrane bound particle is a lentiviral vector. Also provided are related compositions, kits and systems in connection with the provided methods.

### SUMMARY

Provided herein is a method of producing a lipid membrane bound particle, said method comprising culturing host cells comprising one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the pH of the medium during the culturing is maintained at a culture pH of 6.7 to 7.7, wherein the lipid membrane bound particle produced by the method comprises a lipid bilayer and the one or more Paramyxovirus envelope protein or biologically active portion thereof are embedded in the lipid bilayer.

Also provided herein is a method of producing a lipid membrane bound particle, said method comprising culturing host cells transfected with one or more nucleic acids for the production of a lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the pH of the medium during the culturing is maintained at a culture pH that is slightly basic, wherein the lipid membrane bound particle produced by the method comprises a lipid bilayer enclosing a lumen and the one or more Paramyxovirus envelope protein or biologically active portion thereof are embedded in the lipid bilayer.

In some of any of the provided embodiments, at least one of the one or more nucleic acids for the production of the lipid membrane bound particle derived from a virus are stably expressed in the host cell, optionally wherein the one or more nucleic acids are integrated into the chromosome of the host cell. In some of any of the provided embodiments, the nucleic acid encoding the one or more Paramyxovirus envelope protein or a biologically active portion thereof is introduced into the cell, optionally by transfection of the one or more nucleic acids.

Provided herein is a method of producing a lipid membrane bound particle, said method comprising culturing host cells transfected with one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the culturing the pH of the medium during the culturing is maintained at a culture pH of 6.7 to 7.7, wherein the lipid membrane bound particle comprises a lipid bilayer and the one or more Paramyxovirus envelope protein or biologically active portion thereof are embedded in the lipid bilayer.

Also provided herein is a method of producing a lipid membrane bound particle, said method comprising culturing host cells transfected with one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the culturing the pH of the medium during the culturing is maintained at a culture that is slightly basic, wherein the lipid membrane bound particle comprises a lipid bilayer and the one or more Paramyxovirus envelope protein or biologically active portion thereof are embedded in the lipid bilayer.

Also provided herein is a method of culturing a cell, said method comprising culturing host cells comprising one or more nucleic acids for the production of the lipid membrane bound particle derived from a virus, wherein one of the one or more nucleic acids further encodes one or more Paramyxovirus envelope protein or a biologically active portion thereof, and wherein the pH of the medium during the culturing is maintained at a culture pH of 6.7 to 7.7.

Provided herein is a method of culturing a cell, said method comprising culturing host cost cells comprising one or more nucleic acids for the production of the lipid membrane bound particle derived from a virus, wherein one of the one or more nucleic acids further encodes one or more Paramyxovirus envelope protein or a biologically active portion thereof, and wherein the pH of the medium during the culturing is maintained at a culture pH that is slightly basic.

In some of any of the provided embodiments, the one or more nucleic acids for the production of the lipid membrane bound particle derived from a virus are stably expressed in the host cell, optionally wherein the one or more nucleic acids are integrated into the chromosome of the host cell. In some of any of the provided embodiments, the nucleic acid encoding the one or more Paramyxovirus envelope protein or a biologically active portion thereof is introduced into the cell, optionally by transfection of the one or more nucleic acids.

In some of any of the provided embodiments, the pH of the medium during the culturing is maintained at a culture pH of 6.8 to 7.5. In some of any of the provided embodiments, the pH of the medium during the culturing is maintained at a culture pH of 6.9 to 7.4. In some of any of the provided embodiments, the pH of the medium during the culturing is maintained at a culture pH of 7 to 7.3. In some of any of the provided embodiments, the lipid membrane bound particle is a viral-like particle (VLP), or vector particle derived from a retrovirus. In some of any of the provided embodiments, the lipid membrane bound particle is a VLP or vector particle derived from a lentivirus.

Provided herein is a method of producing a lentiviral vector particle, said method comprising culturing host cells transfected with one or more nucleic acids for the production of a lentiviral lipid membrane bound particle, wherein one of the one or more nucleic acids further encodes one or more Paramyxovirus envelope protein or a biologically active portion thereof, and wherein the culturing is carried out in slightly basic medium under conditions for producing the lentiviral lipid membrane bound particle by the host cells.

In some of any of the provided embodiments, the host cell is a mammalian cell, optionally wherein the host cell is selected from the group comprising HEK293 or 293T cells. In some of any of the provided embodiments, the culturing is carried out in medium with a culture pH of 7.05-7.7. In some of any of the provided embodiments, the culturing is carried out in medium with a culture pH of 7.1-7.3.

In some of any of the provided embodiments, the culture pH is allowed to change at a pH set point with a deadband from 0.05, 0.10, 0.15, 0.20, 0.25, 0.30, 0.35, 0.40, 0.45 or 0.50 or any value between any of the foregoing. In some of any of the provided embodiments, the culture pH is selected from: i) pH 7.05±0.05; (ii) pH 7.15±0.05; (iii) pH 7.20±0.05; (iv) pH 7.35±0.05; (v) pH 7.05±0.10; (vi) pH 7.15±0.10; (vii) pH 7.20±0.10; (viii) pH 7.35±0.10; (ix) pH 7.05±0.15; (x) pH 7.15±0.15; (xi) pH 7.20±0.15; or (xii) pH 7.35±0.15.

In some of any of the provided embodiments, the culture pH is at or about 7.05, at or about 7.1, at or about 7.15, at or about 7.2, at or about 7.3, or at or about 7.35.

In some of any of the provided embodiments, the culture pH is at or about 7.1. In some of any of the provided embodiments, the culture pH is at or about 7.2. In some of any of the provided embodiments, the culture pH is at or about 7.3.

In some of any of the provided embodiments, the medium has a dissolved oxygen concentration between 30 and 60 percent saturation. In some of any of the provided embodiments, the medium has a dissolved oxygen concentration of 20, 30, 40, 50, or 60 percent saturation, or any value between any of the foregoing.

In some of any of the provided embodiments, the culturing is carried out in a bioreactor. In some of any of the provided embodiments, the bioreactor is a stirred-tank bioreactor. In some of any of the provided embodiments, the culturing is carried out in a volume of at least 1 L. In some of any of the provided embodiments, the culturing is carried out in a volume of at least 5 L. In some of any of the provided embodiments, the culturing is carried out in a volume between at or about 1 L-5 L, between at or about 5 L-10 L, between at or about 10 L-20 L, between at or about 20 L-50 L, between at or about 50-100 L, or between at or about 100-200 L.

In some of any of the provided embodiments, the method further comprises monitoring the pH of the medium and, optionally adjusting the pH to maintain the culture pH of the medium. In some of any of the provided embodiments, the bioreactor comprises a pH adjustment module, wherein the pH adjustment module monitors the pH of the medium during the culturing. In some of any of the provided embodiments, the bioreactor comprises a pH adjustment module, wherein the pH adjustment module maintains the pH of the medium during the culturing.

In some of any of the provided embodiments, the one or more Paramyxovirus envelope proteins have fusogenic activity. In some of any of the provided embodiments, the native binding tropism of the one or more of the Paramyxovirus envelope proteins is reduced. In some of any of the provided embodiments, the one or more Paramyxovirus envelope proteins is derived from an H protein molecule or a biologically active portion thereof from a Paramyxovirus and/or an HN protein molecule or a biologically active portion thereof from a Paramyxovirus. In some of any of the provided embodiments, the one or more Paramyxovirus envelope proteins comprises an F protein molecule or a biologically active portion thereof and/or a glycoprotein G (G protein) or a biologically active portion thereof. In some of any of the provided embodiments, the one or more Paramyxovirus envelope proteins comprises an F protein molecule or a biologically active portion thereof from a Paramyxovirus and a glycoprotein G (G protein) or a biologically active portion thereof from a Paramyxovirus.

In some of any of the provided embodiments, the paramyxovirus is a henipavirus. In some of any of the provided embodiments, the paramyxovirus is Measles morbillivirus. In some of any of the provided embodiments, the paramyxovirus is a Hendra virus. In some of any of the provided embodiments, the paramyxovirus is Nipah virus.

In some of any of the provided embodiments, the F protein or the biologically active portion thereof is a wild-type Nipah virus F (NiV-F) protein or is a functionally active variant or biologically active portion thereof. In some of any of the provided embodiments, the F protein molecule or a biologically active portion thereof is a NiV-F protein that has the amino acid sequence set forth in SEQ ID NO: 4 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

In some of any of the provided embodiments, the NiV-F protein is a biologically active portion that is truncated and lacks up to 40 contiguous amino acid residues at or near the C-terminus of the wild-type NiV-F protein (SEQ ID NO:4). In some of any of the provided embodiments, the NiV-F protein is a biologically active portion that is truncated at the C-terminus of wild-type NiV-F and has the sequence set forth in any of SEQ ID NOS: 5-15 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 5-15. In some of any of the provided embodiments, the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:4. In some of any of the provided embodiments, the F protein is a NiV-F protein that is a biologically active portion that has a 20 amino acid truncation at or near the C-terminus of the wild-type NiV-F protein (SEQ ID NO:4). In some of any of the provided embodiments, the NiV-F protein has an amino acid sequence set forth in SEQ ID NO:37 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 37. In some of any of the provided embodiments, the F protein is a NiV-F protein that is a biologically active portion thereof that has a 22 amino acid truncation at or near the C-terminus of the wild-type NiV-F protein (SEQ ID NO:4). In some of any of the provided embodiments, the NiV-F protein has an amino acid sequence set forth in SEQ ID NO:32 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32. In some of any of the provided embodiments, the NiV-F protein has an amino acid sequence set forth in SEQ ID NO:33 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 33.

In some of any of the provided embodiments, the F protein or the biologically active portion thereof is a NiV-F protein that is a functionally active variant that comprises a point mutation on an N-linked glycosylation site of the wild-type NiV-F protein (SEQ ID NO:4) or a biologically active potion thereof.

In some of any of the provided embodiments, the F protein or the biologically active portion thereof is a NiV-F protein that is a functionally active variant that comprises: i) a 20 amino acid truncation at or near the C-terminus of the wild-type NiV-F protein (SEQ ID NO:4); and/or ii) a point mutation on an N-linked glycosylation site.

In some of any of the provided embodiments, the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

In some of any of the provided embodiments, the G protein or the biologically active portion thereof is a wild-type Nipah virus G (NiV-G) protein or a Hendra virus G protein or is a functionally active variant or biologically active portion thereof. In some of any of the provided embodiments, the G protein or the biologically active portion thereof is a wild-type NiV-G protein or a functionally active variant or biologically active portion thereof. In some of any of the provided embodiments, the G protein or the biologically active portion thereof is a mutant NiV-G protein that is modified to exhibit reduced native binding tropism. In some of any of the provided embodiments, the G protein or the biologically active portion thereof is a mutant NiV-G protein that exhibits reduced binding to Ephrin B2 or Ephrin B3.

In some of any of the provided embodiments, the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:1. In some of any of the provided embodiments, the NiV-G protein is a biologically active portion that is truncated and lacks up to 40 contiguous amino acid residues at or near the N-terminus of the wild-type NiV-G protein (SEQ ID NO:1). In some of any of the provided embodiments, the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:1, SEQ ID NO:2, or SEQ ID NO: 5.

In some of any of the provided embodiments, the NiV-G protein is a biologically active portion that is truncated at the N-terminus of wild-type NiV-G and has the sequence set forth in any of SEQ ID NOS: 2, 5, or 6 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs 1, 2, or 5. In some of any of the provided embodiments, the G protein molecule or a biologically active portion thereof NiV-G protein has the amino acid sequence set forth in SEQ ID NO: 1 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1. In some of any of the provided embodiments, the G protein or the biologically active portion thereof is a mutant NiV-G protein having the amino acid sequence set forth in SEQ ID NO: 2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:2. In some of any of the provided embodiments, the G protein or the biologically active portion thereof is a mutant NiV-G protein having the amino acid sequence set forth in SEQ ID NO: 5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5.

In some of any of the provided embodiments, the F protein comprises the sequence set forth in SEQ ID NO. 32 and the G protein comprises the sequence set forth in SEQ ID NO. 34. In some of any of the provided embodiments, at least one of the one or more Paramyxovirus envelope proteins are linked to a secondary moiety that is a targeting domain or a functional domain.

In some of any of the provided embodiments, the at least one of the one or more Paramyxovirus is a glycoprotein G (G protein) or a biologically active portion thereof and the G protein or biologically active portion thereof is linked to the secondary moiety. In some of any of the provided embodiments, the secondary moiety is a functional domain and the functional domain is selected from a cytokine, growth factor, hormone, neurotransmitter, receptor, or apoptosis ligand. In some of any of the provided embodiments, the secondary moiety is a targeting domain and the targeting domain is specific for a cell surface receptor on a target cell.

In some of any of the provided embodiments, the targeting domain is a Design ankyrin repeat proteins (DARPin), a single domain antibody (sdAb), a single chain variable fragment (scFv), or an antigen-binding fibronectin type III (Fn3) scaffold. In some of any of the provided embodiments, the at least one of the one or more Paramyxovirus envelope proteins and the secondary moiety are directly linked. In some of any of the provided embodiments, the at least one of the one or more Paramyxovirus envelope proteins and secondary moiety are indirectly linked via a linker. In some of any of the provided embodiments, the linker is a peptide linker. In some of any of the provided embodiments, the peptide linker is (GmS)n(SEQ ID NO: 11), wherein each of m and n is an integer between 1 to 4, inclusive.

In some of any of the provided embodiments, one of the one or more nucleic acids for the production of the lipid membrane bound particle derived from a virus encodes an expression cassette comprising at least one retroviral gene, optionally wherein the at least one retroviral gene is a lentiviral gene. In some of any of the provided embodiments, the at least one retroviral gene is a lentiviral gene selected from the group comprising gag, rev, and/or pol. In some of any of the provided embodiments, the at least one retroviral gene is stably expressed in the host cell, optionally wherein the one or more nucleic acids are integrated into the chromosome of the host cell. In some of any of the provided embodiments, one of the one or more nucleic acids for the production of the lipid membrane bound particle derived from a virus further encodes a transgene. In some of any of the provided embodiments, one of the one or more nucleic acids for the production of the lipid membrane bound particle derived from a virus does not encode VSV-G. In some of any of the provided embodiments, the lipid membrane bound particle derived from a lentivirus or the lentiviral lipid membrane bound particle is a VLP or vector particle derived from HIV-1. In some of any of the provided embodiments, the lipid membrane bound particle derived from a lentivirus or the lentiviral lipid membrane bound particle is a VLP or vector particle derived from gamma-retrovirus (GaL-V).

In some of any of the provided embodiments, the method further comprises purifying the lipid membrane bound particle comprising the one or more Paramyxovirus envelope protein or biologically active portion thereof. In some of any of the provided embodiments, the method further comprises collecting the supernatant from the cell culture, said supernatant containing the lipid membrane bound particle produced by the host cells. In some of any of the provided embodiments, the method further comprises clarification and/or concentration of lipid membrane bound particles. In some of any of the provided embodiments, the method further comprises purifying the lentiviral vector particles comprising the one or more Paramyxovirus envelope protein or biologically active portion thereof. In some of any of the provided embodiments, the method further comprises collecting the supernatant from the cell culture, said supernatant containing the lentiviral vector particles produced by the host cells. In some of any of the provided embodiments, the method further comprises clarification and/or concentration of lentiviral vector particle.

In some of any of the provided embodiments, the clarification and/or concentration is by centrifugation. In some of any of the provided embodiments, the clarification and/or concentration is by dialysis and/or filtration.

Provided herein is a composition, said composition comprising lipid membrane bound particles produced by any of the methods provided herein. Also provided herein is a composition, said composition comprising lentiviral vector particles produced by any of the methods provided herein.

In some of any of the provided embodiments, the ratio of infectious titer to physical titer of the lipid membrane bound particles or lentiviral vector particles is 10,000:1, 5,000:1, 2,500:1, 1,000:1, 100:1 or lower. In some of any of the provided embodiments, the ratio of infectious titer to physical titer of the lipid membrane bound particles or lentiviral vector particles is between 10,000:1 and 100:1, 10,000:1 and 1,1000:1, 10:000:′ and 2,500:1, 1:10,000 and 5000:1, 5,000:1 and 100:1, 5,000:1 and 1000:1, 5000:1 and 2500:1, 2500:1 and 100:1, 2500:1 and 1000:1, or 1000:1 and 100:1. In some of any of the provided embodiments, the ratio of infectious titer to physical titer of the lipid membrane bound particles or lentiviral vector particles is between 10,000:1 and 1000:1. In some of any of the provided embodiments, the ratio of infectious titer to physical titer of the lipid membrane bound particles or lentiviral vector particles is between 10,000:1 and 5000:1.

Provided herein is a cell culture system, wherein said system comprises a vessel comprising cell culture medium and host cells, said vessel further comprising a pH monitoring module contacting cell culture medium comprising host cells transfected with one or more nucleic acids for the production of a lipid membrane bound particle derived from a virus, wherein at least one the one of the one or more nucleic acids further encodes an envelope protein derived from a Paramyxovirus.

In some of any of the provided embodiments, the lipid membrane bound particle is a retroviral vector particle, optionally a lentiviral vector particle.

Provided herein is a cell culture system, wherein said system comprises a vessel comprising cell culture medium and host cells, said vessel further comprising a pH monitoring module contacting the cell culture medium comprising host cells transfected with one or more nucleic acids for the production of a lentiviral vector particle, wherein the one or more nucleic acids further encodes an envelope protein derived from a Paramyxovirus. Also provided herein is a cell culture system, wherein said system comprises a vessel comprising at least 5 L of cell culture medium comprising host cells transfected with one or more nucleic acids for the production of a lipid membrane bound particle derived from a virus, wherein the at least one of the one or more nucleic acids further encodes an envelope protein derived from a Paramyxovirus.

In some of any of the provided embodiments, said vessel comprises a bioreactor. In some of any of the provided embodiments, the cell culture system is for culturing the host cells under conditions for producing the lipid membrane bound particle by the host cells. In some of any of the provided embodiments, the system further comprises a pH adjustment module, wherein the pH adjustment module monitors the pH of the medium during the culturing. In some of any of the provided embodiments, the system further comprises a pH adjustment module, wherein the pH adjustment module maintains the pH of the medium during the culturing.

In some of any of the provided embodiments, the medium is maintained at a pH of 6.7 to 7.7. In some of any of the provided embodiments, the medium is maintained at a pH of 7.1-7.3. In some of any of the provided embodiments, the medium has a pH of at or about 7.1. In some of any of the provided embodiments, the medium has a pH of at or about 7.2. In some of any of the provided embodiments, the medium has a pH of at or about 7.3.

In some of any of the provided embodiments, the medium is maintained at a pH with a deadband from 0.05, 0.10, 0.15, 0.20, 0.25, 0.30, 0.35, 0.40, 0.45 or 0.50 or any value between any of the foregoing. In some of any of the provided embodiments, the medium is maintained at a pH selected from: (i) pH 7.05±0.05; (ii) pH 7.15±0.05; (iii) pH 7.20±0.05; (iv) pH 7.35±0.05; (v) pH 7.05±0.10; (vi) pH 7.15±0.10; (vii) pH 7.20±0.10; (viii) pH 7.35±0.10; (ix) pH 7.05±0.15; (x) pH 7.15±0.15; (xi) pH 7.20±0.15; or (xii) pH 7.35±0.15. In some of any of the provided embodiments, the culture pH is at or about 7.05, at or about 7.1, at or about 7.15, at or about 7.2, at or about 7.3, at or about 7.35.

In some of any of the provided embodiments, the medium has a dissolved oxygen concentration between 40 and 60 percent saturation. In some of any of the provided embodiments, the system further comprises a glucose monitor. In some of any of the provided embodiments, the system further comprises a module for controlling stirring speed.

In some of any of the provided embodiments, the one or more Paramyxovirus envelope proteins have fusogenic activity. In some of any of the provided embodiments, the native binding tropism of the one or more of the Paramyxovirus envelope proteins is reduced. In some of any of the provided embodiments, the one or more Paramyxovirus envelope proteins is derived from an H protein molecule or a biologically active portion thereof from a Paramyxovirus and/or an HN protein molecule or a biologically active portion thereof from a Paramyxovirus. In some of any of the provided embodiments, the one or more Paramyxovirus envelope proteins comprises an F protein molecule or a biologically active portion thereof and/or a glycoprotein G (G protein) or a biologically active portion thereof. In some of any of the provided embodiments, the one or more Paramyxovirus envelope proteins comprises an F protein molecule or a biologically active portion thereof from a Paramyxovirus and a glycoprotein G (G protein) or a biologically active portion thereof from a Paramyxovirus.

In some of any of the provided embodiments, the paramyxovirus is a henipavirus. In some of any of the provided embodiments, the paramyxovirus is Measles morbillivirus. In some of any of the provided embodiments, the paramyxovirus is a Hendra virus. In some of any of the provided embodiments, the paramyxovirus is Nipah virus. In some of any of the provided embodiments, the F protein or the biologically active portion thereof is a wild-type Nipah virus F (NiV-F) protein or is a functionally active variant or biologically active portion thereof.

In some of any of the provided embodiments, the F protein molecule or a biologically active portion thereof is a NiV-F protein that has the amino acid sequence set forth in SEQ ID NO: 4 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4. In some of any of the provided embodiments, the NiV-F protein is a biologically active portion that is truncated and lacks up to 40 contiguous amino acid residues at or near the C-terminus of the wild-type NiV-F protein (SEQ ID NO:4). In some of any of the provided embodiments, the NiV-F protein is a biologically active portion that is truncated at the C-terminus of wild-type NiV-F and has the sequence set forth in any of SEQ ID NO. 32 or SEQ ID NO. 33 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO. 32 or SEQ ID NO. 33. In some of any of the provided embodiments, the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:4. In some of any of the provided embodiments, the F protein is a NiV-F protein that is a biologically active portion that has a 20 amino acid truncation at or near the C-terminus of the wild-type NiV-F protein (SEQ ID NO:4). In some of any of the provided embodiments, the NiV-F protein has an amino acid sequence set forth in SEQ ID NO:37 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 37. In some of any of the provided embodiments, the F protein is a NiV-F protein that is a biologically active portion thereof that has a 22 amino acid truncation at or near the C-terminus of the wild-type NiV-F protein (SEQ ID NO:4). In some of any of the provided embodiments, the NiV-F protein has an amino acid sequence set forth in SEQ ID NO:38 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 38. In some of any of the provided embodiments, the NiV-F protein has an amino acid sequence set forth in SEQ ID NO:36 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36.

In some of any of the provided embodiments, the F protein or the biologically active portion thereof is a NiV-F protein that is a functionally active variant that comprises a point mutation on an N-linked glycosylation site of the wild-type NiV-F protein (SEQ ID NO:4) or a biologically active potion thereof. In some of any of the provided embodiments, the F protein or the biologically active portion thereof is a NiV-F protein that is a functionally active variant that comprises: i) a 20 amino acid truncation at or near the C-terminus of the wild-type NiV-F protein (SEQ ID NO:4); and/or ii) a point mutation on an N-linked glycosylation site. In some of any of the provided embodiments, the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

In some of any of the provided embodiments, the G protein or the biologically active portion thereof is a wild-type Nipah virus G (NiV-G) protein or a Hendra virus G protein or is a functionally active variant or biologically active portion thereof. In some of any of the provided embodiments, the G protein or the biologically active portion thereof is a wild-type NiV-G protein or a functionally active variant or biologically active portion thereof. In some of any of the provided embodiments, the G protein or the biologically active portion thereof is a mutant NiV-G protein that is modified to exhibit reduced native binding tropism. In some of any of the provided embodiments, the G protein or the biologically active portion thereof is a mutant NiV-G protein that exhibits reduced binding to Ephrin B2 or Ephrin B3. In some of any of the provided embodiments, the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:1. In some of any of the provided embodiments, the NiV-G protein is a biologically active portion that is truncated and lacks up to 40 contiguous amino acid residues at or near the N-terminus of the wild-type NiV-G protein (SEQ ID NO:1). In some of any of the provided embodiments, the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:1, SEQ ID NO:2, or SEQ ID NO: 5. In some of any of the provided embodiments, the NiV-G protein is a biologically active portion that is truncated at the N-terminus of wild-type NiV-G and has the sequence set forth in any of SEQ ID NOS: 1, 2, or 5 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 1, 2, or 5. In some of any of the provided embodiments, the G protein molecule or a biologically active portion thereof NiV-G protein has the amino acid sequence set forth in SEQ ID NO: 1 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1. In some of any of the provided embodiments, the G protein or the biologically active portion thereof is a mutant NiV-G protein having the amino acid sequence set forth in SEQ ID NO: 2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:2. In some of any of the provided embodiments, the G protein or the biologically active portion thereof is a mutant NiV-G protein having the amino acid sequence set forth in SEQ ID NO: 5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5.

In some of any of the provided embodiments, the F protein comprises the sequence set forth in SEQ ID NO. 32 and the G protein comprises the sequence set forth in SEQ ID NO. 34.

In some of any of the provided embodiments, at least one of the one or more Paramyxovirus envelope proteins are linked to a secondary moiety that is a targeting domain or a functional domain. In some of any of the provided embodiments, the at least one of the one or more Paramyxovirus is a glycoprotein G (G protein) or a biologically active portion thereof and the G protein or biologically active portion thereof is linked to the secondary moiety. In some of any of the provided embodiments, the secondary moiety is a functional domain and the functional domain is selected from a cytokine, growth factor, hormone, neurotransmitter, receptor, or apoptosis ligand. In some of any of the provided embodiments, the secondary moiety is a targeting domain and the targeting domain is specific for a cell surface receptor on a target cell. In some of any of the provided embodiments, the targeting domain is a Design ankyrin repeat proteins (DARPin), a single domain antibody (sdAb), a single chain variable fragment (scFv), a VHH antibody (nanobody), or an antigen-binding fibronectin type III (Fn3) scaffold. In some of any of the provided embodiments, the at least one of the one or more Paramyxovirus envelope proteins and the secondary moiety are directly linked. In some of any of the provided embodiments, the at least one of the one or more Paramyxovirus envelope proteins and secondary moiety are indirectly linked via a linker. In some of any of the provided embodiments, the linker is a peptide linker. In some of any of the provided embodiments, the peptide linker is (GₘS)ₙ(SEQ ID NO: 11), wherein each of m and n is an integer between 1 to 4, inclusive.

Provided herein is a method of producing a lipid membrane bound particle, said method comprising culturing host cost cells in a cell culture system, wherein said host cells comprise one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid bilayer.

Provided herein is a method of producing a lipid membrane bound particle, said method comprising culturing host cost cells in a cell culture system, wherein said host cells are transfected with one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid bilayer.

Provided herein is a method of culturing a cell, said method comprising culturing host cells in a cell culture system, wherein said host cells are transfected with one or more nucleic acids for the production of a lipid membrane bound particle derived from a virus, wherein one of the one or more nucleic acids further encodes one or more Paramyxovirus envelope protein or a biologically active portion thereof, and wherein the culturing is carried out in medium with a culture pH that is slightly basic under conditions for producing the lipid membrane bound particle by the host cells, wherein the lipid membrane bound particle comprises a lipid bilayer enclosing a lumen and the one or more Paramyxovirus envelope protein or biologically active portion thereof are embedded in the lipid bilayer.

### BRIEF DESCRIPTION OF THE DRAWINGS

FIG. 1A depicts the functional titer of VSV-G pseudotyped vector across a range of pH, while cell specific productivity is shown for the same VSV-G pseudotyped vector in FIG. 1B. FIG. 1C shows functional titer observed with production of CD8-retargeted Nipah pseudotyped vector, cell specificity for this vector is depicted in FIG. 1D.

FIG. 2 depicts the predicted effect of pH and DO on the ratio of physical titer to functional titer for production of a CD8-retargeted Nipah pseudotyped vector.

Mean functional titer for various exemplary culture conditions is shown in FIG. 3A. FIG. 3B depcits mean infectivity as VLP/TU for exemplary culture conditions.

### DETAILED DESCRIPTION

Provided herein are methods and compositions of producing a lipid membrane bound particle (e.g. lentiviral vector) containing one or more Paramyxovirus envelope protein expressed on the lipid bilayer. In some embodiments, the one or more Paramyxovirus envelope protein includes a Nipah F protein and a Nipah G protein or biologically active portions of such proteins. In some embodiments, the provided method includes culturing permissive host cells with at least one plasmid for the production of a lipid membrane particle derived from a lentivirus wherein one of the at least one plasmid further encodes the one or more Paramyxovirus envelope protein or a biologically active portion thereof. In some of any embodiments, the culturing of permissive host cells is carried out in medium with a pH of 6.7 to 7.7 under conditions for producing the lipid membrane bound particle by the host cells. In some of any embodiments, the one or more Paramyxovirus envelope protein or biologically active portion thereof are embedded in the lipid bilayer of the produced lipid membrane bound particle. Also provided herein are lipid membrane bound particles and compositions thereof containing any of the provided lipid membrane produced by any of the methods herein.

The provided embodiments relate to methods for improving production of certain lipid particles, such as lentiviral vectors. Recent developments in the fields of gene therapy and gene engineering using recombinant membrane bound lipid particles, such as viral vectors and virus-like particles, have created a large unmet need for large scale production of specific clinical quality particles. One such family of viral vectors is the genus of lentiviruses within the retrovirus family of viruses. Lentiviral vectors used in gene therapy or engineering applications are conventionally manufactured by calcium phosphate transfection of adherent cells which require fetal bovine serum in the culture media, with a lentiviral construct DNA system (Merten et al, 2011, Hum Gene Ther. 22(3):343-56). Current methods for production of these vectors include adherent cell cultures in roller bottles, cell factories, or cell cubes. The presence of an animal derived component in the culture constitutes a safety risk that limits the GMP compliance of these methods. In addition these culture methods are severely limited in terms of scale up and is not adapted to the production of large amounts of lipid membrane bound particles required for therapeutic, commercial and/or industrial applications of gene therapy.

In some cases, methods of suspension culture can allow for increased production of lipid membrane bound particles. For example, the production of viral vectors by transient transfection in suspension cultures in the absence of serum has recently been described. Although limited in volume to only 3 liters, Ansorge et al. have proposed a process for the production of lentiviruses by transient transfection of suspension-grown HEK293SF-3F6 cells in perfusion cultures (Ansorge et al, 2009, J Gene Med, 11: 868-876). In such processes, different conditions like agitation of the culture, temperature, glucose availability, total biomass, pH, and dissolved oxygen are parameters that may impact cellular viability and lipid membrane bound particle production. Thus, there remains a need for improved methods of viral vector production. Further, there is limited information is available for the large scale production of membrane bound lipid particles with one or more Paramyxovirus envelope protein embedded in the lipid bilayer.

The present disclosure relates to an optimized method for the production of lipid particles containing a Paramyxovirus envelope protein, such as Nipah Virus (NiV) proteins NiV-G and NiV-F. For instance, provided are methods for the efficient production of NiV pseudotyped Lentiviral Vectors (LVs). It has been surprisingly found that certain culture conditions, such as pH conditions, in the cell culture substantially affect total particle production, functional particle production, and the ratio of the total to functional particles of NiV pseudotyped LVs. In particular, pH conditions in the culture impact functional titer and cell specific productivity of produced lentiviral vector preparations. For instance, optimal functional titer production of NiV pseudotyped vectors was achieved under slightly basic conditions, such as between pH 7.1 and 7.3. In addition, cell culture medium with particular dissolved oxygen concentration, such as between 40 and 60 percent air saturation, also can impact NiV vector production. This was surprising because functional particle production of the more common VSV-G pseudotyped lentiviral vector can be achieved under mildly acidic conditions (pH 6.8-6.9) (Holic et al. Hum Gene Ther Clin Dev. 2014; 25(3):178-185; U.S. Pat. No. 10,125,352).

In some embodiments, the provided methods can be conducted in scalable stirred tank bioreactors. Thus, the provided methods can be adapted for scale to provide an optimized means to produce large quantities of NiV pseudotyped viral vectors. The provided embodiments provide for a simple and scalable method to produce Nipah Virus pseudotyped lentiviral vectors with enhanced quality (infectivity) and quantity (functional titer) compared to traditional methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Production of Paramyxovirus (e.g. Nipah Protein) Lipid Particles

The provided methods include methods of producing a lipid membrane bound particle containing one or more Paramyxovirus envelope protein embedded in the lipid bilayer. In some embodiments, the one or more Paramyxovirus envelope protein act as a fusogen to support or mediate fusion of the produced lipid particle with a target cells. In particular embodiments, the one or more Paramyxovirus envelope protein are Nipah virus envelope proteins NiV-F and NiV-G, including such proteins that are retargeted to permit delivery of genes to desired target cells. Features of the methods and lipid membrane bound particles are described in the following sections.

In some embodiments, the provided methods include culturing host cells containing one or more nucleic acids for the production of a lipid membrane bound particle (e.g. lentiviral vector) under conditions for producing the lipid membrane bound particle by the host cells, wherein one of the at least one nucleic acid further encodes one or more Paramyxovirus envelope protein (e.g. NiV—F and NiV-G) or a biologically active portion thereof. In some embodiments, the one more nucleic acids for the production of a lipid membrane bound particle are derived from a virus, such as a lentivirus. Thus, in some embodiments, the lipid membrane bound particles are lentiviral vectors and are pseudotyped with the one or more Paramyxovirus envelope protein. In some embodiments, the produced lipid membrane bound particle includes a lipid bilayer enclosing a lumen and the one or more Paramyxovirus envelope protein or biologically active portion thereof are embedded in the lipid bilayer.

In some embodiments, the provided methods include introducing one or more plasmids into the host cells prior to the culturing of the host cells. In some embodiments, the host cells for culturing may carry the one or more nucleic acids having been previously introduced with the one or more nucleic acids, such as under conditions for their stable expression in the host cell. In some embodiments, the one or more nucleic acids may include packaging plasmids, transfer plasmids and nucleic acids encoding the one or more Paramyxovirus envelope protein(s) and, in some cases, a nucleic acid encoding an exogenous agent. In some embodiments, the nucleic acid encoding the Paramyxovirus envelope protein(s) is contained within the packaging plasmid or is carried in a separate plasmid. In some embodiments, the nucleic acid encoding the exogenous agent is within the transfer plasmid or is carried on another separate plasmid. In some embodiments, the one or more plasmids can be introduced into the host cells by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones.

In some of any embodiments, the introducing one or more plasmids into the host cells (e.g., a transfection) is done within at or about 6 hours, 12 hours, 24 hours, 36 hours, 48 hours, or 72 hours following cell seeding (i.e., cultivation as described in Section I.D), or any time period between any of the foregoing. In some of any embodiments, the introducing one or more plasmids into the host cells is done between 6-12 hours, 6-24 hours, 6-36 hours, 6-48 hours, 6-72 hours, 12-36 hours, 12-48 hours, 12-72 hours, 24-36 hours, 24-48 hours, 24-72 hours, 36-48 hours, 36-72 hours, or 48-72 hours following cell seeding, each inclusive.

In some of any embodiments, the introducing one or more plasmids into the host cells (e.g., a transfection) is done once a threshold target cell density is reached following cell seeding (i.e., cultivation as described in Section I.D). In some of any embodiments, the threshold target cell density is at or about 2.0×10⁶, 2.5×10⁶, 3.0×10⁶, 3.5×10⁶, or 4.0×10⁶′ or any target cell density between any of the foregoing.

In some of any embodiments, the target cell density is between 2.0×10⁶ and 2.5×10⁶, 2.0×10⁶ and 3.0×10⁶, 2.0×10⁶ and 3.5×10⁶, 2.0×10⁶ and 4.0×10⁶, 2.5×10⁶ and 3.0×10⁶, 2.5×10⁶ and 3.5×10⁶, 2.5×10⁶ and 4.0×10⁶, 3.0×10⁶ and 3.5×10⁶, 3.0×10⁶ and 4.0.×10⁶, or 4.0×10⁶ and 4.5×10⁶, each inclusive.

In some of any embodiments, the host cells are cultured following the introduction of the one or more plasmids into the same. In some of any embodiments, the host cells are cultured in media that is supplemented with one or more factors following the introduction of the one or more plasmids into the same. In some of any embodiments, the host cells are cultured in media that is supplemented with Sodium butyrate and/or glucose.

In some of any embodiments, Sodium butyrate and/or glucose are supplemented with cell media. In some embodiments, the supplements, such as sodium butyrate and/or glucose, are added to the cell media within at or about 6 hours, 12 hours, 24 hours, 36 hours, 48 hours, or 72 hours, or any time period between any of the foregoing, following the introduction of one or more plasmids (e.g., transfection). In some of any embodiments, the supplements, such as sodium butyrate and/or glucose, are added to the cell media (supplemented with cell media) at a time between 6-12 hours, 6-24 hours, 6-36 hours, 6-48 hours, 6-72 hours, 12-36 hours, 12-48 hours, 12-72 hours, 24-36 hours, 24-48 hours, 24-72 hours, 36-48 hours, 36-72 hours, or 48-72 hours, each inclusive, following the introduction of one or more plasmids, each inclusive.

In some embodiments, the methods include culturing host cells wherein the pH of the medium during the culturing, or at least a portion of the culturing, is maintained at a desired pH. In provided embodiments, the pH of the medium during the culturing is maintained at a pH of 6.7 to 7.7. In some embodiments, the pH is a slightly basic pH. For instance, in some embodiments, the pH is at or about 7.1 to at or about 7.3. In some embodiments, the pH is at or about 7.1, 7.2, 7.3.

In some embodiments, the provided methods can be carried out or performed in a cell culture system. For instance, in some embodiments, host cells that carry or are introduced with the one or more viral nucleic acids can be grown in a cell culture system, such as described herein in Section II.

In some embodiments, the lipid membrane bound particle can be a viral particle, a virus-like particle, a lentivirus, a viral vector, or a lentiviral vector, a viral based particle, or a virus like particle (VLP). Also provided herein are the lipid membrane bound particles and compositions thereof according to the provided embodiments and methods.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid membrane particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the lipid membrane bound particle packages nucleic acids from host cells during the expression process. In particular embodiments, a polynucleotide or polypeptide is encapsulated within the lumen of the lipid membrane bound particle in which the lipid particle contains a lipid bilayer, a lumen surrounded by the lipid bilayer. In some embodiments, the culturing of the host cells containing nucleic derived from a virus is under conditions for producing a lipid particle by the host cells. Viral vectors, and especially retroviral vectors such as those derived from lentivirus, have become the most widely used method for inserting genes into mammalian cells, e.g., any of the cells disclosed above. Other viral vectors can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the lipid membrane bound particles produced by the provided methods can be a viral particle, a virus-like particle, a lentivirus, a viral vector, or a lentiviral vector, a viral based particle, or a virus like particle (VLP). In some embodiments, the lipid membrane bound particles produced by the provided methods is a virus based vector particle. In some embodiments, the virus based vector particles are lentivirus. In some embodiments, the lentiviral vector particle is Human Immunodeficiency Virus-1 (HIV-1). In some embodiments, the nucleic acids do not encode any genes involved in virus replication. In particular embodiments, the lipid membrane bound particle is a virus-like particle, e.g. retrovirus-like particle such as a lentivirus-like particle, that is replication defective.

### A. Paramyxovirus Envelope Protein and Methods and Genes for Particle Targeting and Retargeting

In some embodiments of the provided methods one of the one or more nucleic acids further encodes one or more Paramyxovirus envelope protein or a biologically active portion thereof, In some embodiments, the Paramyxovirus envelope protein or a biologically active portion thereof harbor the attachment and/or fusion glycoproteins and are capable of binding to target cells and delivering the vehicle contents to the cytoplasm of the target cells.

In some embodiments, the paramyxovirus envelope protein binds to sialic acid receptors, and hence the corresponding derivative lipid membrane particles can deliver their contents generically to nearly any kind of cell that expresses sialic acid receptors. In some embodiments, the paramyxovirus envelope protein binds to protein receptors, and hence the corresponding lipid membrane particle produced by the method can deliver their contents to any cell that expresses that protein receptor. For example, Nipah virus binds to protein receptors, and hence the corresponding vehicles have a specificity that matches the natural tropisms for this virus and its surface proteins.

In some embodiments, the nucleic acid encoding the Paramyxovirus is modified with a targeting moiety to specifically bind to a target molecule on a target cells. Hence, the lipid membrane bound particle produced by the provided method further comprises a vector-surface targeting moiety which specifically binds to a target ligand. In some embodiments, the targeting moiety can be any targeting protein, including but not necessarily limited to antibodies and antigen binding fragments thereof, receptor ligands, and other approaches that will be apparent to those skilled in the art given the benefit of the present disclosure. In some embodiments, the particle-surface targeting moiety is a polypeptide.

In some embodiments, the Paramyxovirus envelope protein is a fusogen that support or mediate fusion of the produced lipid particle with a target cell. In some embodiments, the vector particle, e.g. viral vector or viral-like particle, contains an exogenous or overexpressed fusogen. In some embodiments, the fusogen is disposed in the lipid bilayer. In some embodiments, the fusogen facilitates the fusion of the lipid membrane bound particle to a membrane. In some embodiments, the membrane is a plasma cell membrane. In some embodiments, the lipid particle, such as a viral vector or VLP, comprising the fusogen integrates into the membrane into a lipid bilayer of a target cell. In some embodiments, the fusogen results in mixing between lipids in the lipid particle and lipids in the target cell. In some embodiments, the fusogen results in formation of one or more pores between the interior of the vector vehicle particle and the cytosol of the target cell. In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a Tupaia paramyxovirus F protein, a paramyxovirus F protein, a Hendra virus F protein, a Henipavirus F protein, a Morbilivirus F protein, a respirovirus F protein, a Sendai virus F protein, a canine distemper virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the sequence of the F protein is as set forth in SEQ ID. NO 4. In some embodiments, the F protein is truncated and lacks up to 42 contiguous amino acids, such as up to 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the C-terminus of SEQ ID. NO 4.

In some embodiments, the sequence of the HN protein is as set forth in SEQ ID. NO 47. In some embodiments, the HN protein is modified, such as by modification to the C-terminal domain. In some embodiments, the sequence of the HN protein is as set forth in SEQ ID. NO 48.

In some embodiments, the fusogen is a F and/or HN protein from the murine parainfluenza virus type 1 (See e.g., U.S. patent Ser. No. 10/704,061).

### 1. F Proteins

In some embodiments, the lipid membrane bound particle-surface comprises a protein with a hydrophobic fusion peptide domain. In some embodiments, the protein with a hydrophobic fusion peptide domain may be an envelope glycoprotein F protein of the Paramyxoviridae family (i.e., a paramyxovirus F protein). In some embodiments, the envelope glycoprotein F protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mojiang virus F protein or a bat Paramyxovirus F protein or a biologically active portion thereof.

Table 1 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:16). Following cleavage of the signal peptide, the mature F₀ (e.g. SEQ ID NO:17) is transported to the cell surface, then endocytosed and cleaved by cathepsin L into the mature fusogenic subunits F1 and F2. The F1 and F2 subunits are associated by a disulfide bond and recycled back to the cell surface. The F1 subunit contains the fusion peptide domain located at the N terminus of the F1 subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93(13):e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 1
F proteins

| | | | SEQ ID |
|---|---|---|---|
| | | | (without |
| Full Gene | | SEQ | signal |
| Name | Sequence | ID | sequence) |
| Hendra virus | MATQEVRLKCLLCGIIVLVLSLEGLGILHYEKLSKIGLVKGITR | 16 | 17 |
| F Protein | KYKIKSNPLTKDIVIKMIPNVSNVSKCTGTVMENYKSRLTGIL | | |
| | SPIKGAIELYNNNTHDLVGDVKLAGVVMAGIAIGIATAAQITA | | |
| | GVALYEAMKNADNINKLKSSIESTNEAVVKLQETAEKTVYVL | | |
| | TALQDYINTNLVPTIDQISCKQTELALDLALSKYLSDLLFVFGP | | |
| | NLQDPVSNSMTIQAISQAFGGNYETLLRTLGYATEDFDDLLES | | |
| | DSIAGQIVYVDLSSYYIIVRVYFPILTEIQQAYVQELLPVSENN | | |
| | DNSEWISIVPNFVLIRNTLISNIEVKYCLITKKSVICNQDYATP | | |
| | MTASVRECLTGSTDKCPRELVVSSHVPRFALSGGVLFANCISV | | |
| | TCQCQTTGRAISQSGEQTLLMIDNTTCTTVVLGNIIISLGKYLG | | |
| | SINYNSESIAVGPPVYTDKVDISSQISSMNQSLQQSKDYIKEAQ | | |
| | KILDTVNPSLISMLSMIILYVLSIAALCIGLITFISFVIVEKKRGN | | |
| | YSRLDDRQVRPVSNGDLYYIGT | | |
| Nipah virus F | MVVILDKRCYCNLLILILMISECSVGILHYEKLSKIGLVKGVTR | 18 | 19 |
| Protein | KYKIKSNPLTKDIVIKMIPNVSNMSQCTGSVMENYKTRLNGIL | | |
| | TPIKGALEIYKNNTHDLVGDVRLAGVIMAGVAIGIATAAQITA | | |
| | GVALYEAMKNADNINKLKSSIESTNEAVVKLQETAEKTVYVL | | |
| | TALQDYINTNLVPTIDKISCKQTELSLDLALSKYLSDLLFVFGP | | |
| | NLQDPVSNSMTIQAISQAFGGNYETLLRTLGYATEDFDDLLES | | |
| | DSITGQIIYVDLSSYYIIVRVYFPILTEIQQAYIQELLPVSENNDN | | |
| | SEWISIVPNFILVRNTLISNIEIGFCLITKRSVICNQDYATPMTN | | |
| | NMRECLTGSTEKCPRELVVSSHVPRFALSNGVLFANCISVTCQ | | |
| | CQTTGRAISQSGEQTLLMIDNTTCPTAVLGNVIISLGKYLGSV | | |
| | NYNSEGIAIGPPVFTDKVDISSQISSMNQSLQQSKDYIKEAQRL | | |
| | LDTVNPSLISMLSMIILYVLSIASLCIGLITFISFIIVEKKRNTYSR | | |
| | LEDRRVRPTSSGDLYYIGT | | |
| Cedar Virus F | MSNKRTTVLIIISYTLFYLNNAAIVGFDFDKLNKIGVVQGRVL | 20 | 21 |
| Protein | NYKIKGDPMTKDLVLKFIPNIVNITECVREPLSRYNETVRRLL | | |
| | LPIHNMLGLYLNNTNAKMTGLMIAGVIMGGIAIGIATAAQITA | | |
| | GFALYEAKKNTENIQKLTDSIMKTQDSIDKLTDSVGTSILILNK | | |
| | LQTYINNQLVPNLELLSCRQNKIEFDLMLTKYLVDLMTVIGPN | | |
| | INNPVNKDMTIQSLSLLFDGNYDIMMSELGYTPQDFLDLIESK | | |
| | SITGQIIYVDMENLYVVIRTYLPTLIEVPDAQIYEFNKITMSSN | | |
| | GGEYLSTIPNFILIRGNYMSNIDVATCYMTKASVICNQDYSLP | | |
| | MSQNLRSCYQGETEYCPVEAVIASHSPRFALTNGVIFANCINTI | | |
| | CRCQDNGKTITQNINQFVSMIDNSTCNDVMVDKFTIKVGKY | | |
| | MGRKDINNINIQIGPQIIIDKVDLSNEINKMNQSLKDSIFYLREA | | |
| | KRILDSVNISLISPSVQLFLIIISVLSFIILLIIIVYLYCKSKHSYKY | | |
| | NKFIDDPDYYNDYKRERINGKASKSNNIYYVGD | | |
| Mojiang virus, | MALNKNMFSSLFLGYLLVYATTVQSSIHYDSLSKVGVIKGLT | 22 | 23 |
| Tongguan 1 F | YNYKIKGSPSTKLMVVKLIPNIDSVKNCTQKQYDEYKNLVRK | | |
| Protein | ALEPVKMAIDTMLNNVKSGNNKYRFAGAIMAGVALGVATA | | |
| | ATVTAGIALHRSNENAQAIANMKSAIQNTNEAVKQLQLANK | | |
| | QTLAVIDTIRGEINNNIIPVINQLSCDTIGLSVGIRLTQYYSEIIT | | |
| | AFGPALQNPVNTRITIQAISSVFNGNFDELLKIMGYTSGDLYEI | | |
| | LHSELIRGNIIDVDVDAGYIALEIEFPNLTLVPNAVVQELMPIS | | |
| | YNIDGDEWVTLVPRFVLTRTTLLSNIDTSRCTITDSSVICDNDY | | |
| | ALPMSHELIGCLQGDTSKCAREKVVSSYVPKFALSDGLVYAN | | |
| | CLNTICRCMDTDTPISQSLGATVSLLDNKRCSVYQVGDVLISV | | |
| | GSYLGDGEYNADNVELGPPIVIDKIDIGNQLAGINQTLQEAED | | |
| | YIEKSEEFLKGVNPSIITLGSMVVLYIFMILIAIVSVIALVLSIKL | | |
| | TVKGNVVRQQFTYTQHVPSMENINYVSH | | |
| Bat | MKKKTDNPTISKRGHNHSRGIKSRALLRETDNYSNGLIVENL | 24 | 25 |
| Paramyxoviru | VRNCHHPSKNNLNYTKTQKRDSTIPYRVEERKGHYPKIKHLI | | |
| s Eid_hel/GH- | DKSYKHIKRGKRRNGHNGNIITIILLLILILKTQMSEGAIHYETL | | |
| M74a/GHA/2 | SKIGLIKGITREYKVKGTPSSKDIVIKLIPNVTGLNKCTNISMEN | | |
| 009 F protein | YKEQLDKILIPINNIIELYANSTKSAPGNARFAGVIIAGVALGV | | |
| | AAAAQITAGIALHEARQNAERINLLKDSISATNNAVAELQEAT | | |
| | GGIVNVITGMQDYINTNLVPQIDKLQCSQIKTALDISLSQYYSE | | |
| | ILTVFGPNLQNPVTTSMSIQAISQSFGGNIDLLLNLLGYTANDL | | |
| | LDLLESKSITGQITYINLEHYFMVIRVYYPIMTTISNAYVQELIK | | |
| | ISFNVDGSEWVSLVPSYILIRNSYLSNIDISECLITKNSVICRHDF | | |
| | AMPMSYTLKECLTGDTEKCPREAVVTSYVPRFAISGGVIYAN | | |
| | CLSTTCQCYQTGKVIAQDGSQTLMMIDNQTCSIVRIEEILISTG | | |
| | KYLGSQEYNTMHVSVGNPVFTDKLDITSQISNINQSIEQSKFY | | |
| | LDKSKAILDKINLNLIGSVPISILFIIAILSLILSIITFVIVMIIVRRY | | |
| | NKYTPLINSDPSSRRSTIQDVYIIPNPGEHSIRSAARSIDRDRD | | |

In some embodiments, the F protein is encoded by a nucleotide sequence that encodes the sequence set forth by any one of SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, SEQ ID NO:24, or SEQ ID NO:25 or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, SEQ ID NO:24, or SEQ ID NO:25.

In particular embodiments, the F protein or the functionally active variant or biologically active portion thereof retains fusogenic activity in conjunction with a Henipavirus G protein, such as a G protein set forth in Section I.A.2. Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L(e.g. corresponding to the cleavage site between amino acids 109-110 of SEQ ID NO:18).

In some embodiments, a NiV-F, such as a mutant or truncated NiV-F, of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:18) to produce two chains that can be linked by disulfide bond. In some embodiments, the NiV-F, such as wild-type NiV-F or a truncated or mutated NiV-F protein, is produced or encoded as an F₀ precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the F₀ precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:18 or truncated or mutant sequence thereof, and an F2 corresponding to amino acid residues 27-109 of NiV-F set forth in SEQ ID NO:18.

In particular embodiments, the F protein has the sequence of amino acids set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25., or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25.

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (HeV) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25,

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 16-25. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO: 36. The encoding nucleic acid can encode a signal peptide sequence that has the sequence MVVILDKRCY CNLLILILMI SECSVG (SEQ ID NO: 26). In some embodiments, the F protein has the sequence set forth in SEQ ID NO:18. In some examples, the F protein is cleaved into an F1 subunit comprising the sequence set forth in SEQ ID NO:28 and an F2 subunit comprising the sequence set forth in SEQ ID NO: 27.

In some embodiments, the F protein is a NiV-F protein that is encoded by a sequence of nucleotides encoding the sequence set forth in SEQ ID NO:18, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 18. In some embodiments, the NiV-F-protein has the sequence of set forth in SEQ ID NO: 19, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 19. In particular embodiments, the F protein or the functionally active variant or biologically active portion thereof retains the cleavage site cleaved by cathepsin L.

In some embodiments, the F protein or the functionally active variant or the biologically active portion thereof includes an F1 subunit that has the sequence set forth in SEQ ID NO: 28, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28.

In some embodiments, the F protein or the functionally active variant or biologically active portion thereof includes an F2 subunit that has the sequence set forth in SEQ ID NO: 27, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:27.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:28). In some embodiments, the mutant NiV-F protein comprises an amino acid sequence set forth in SEQ ID NO:29. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 29. In some embodiments, the mutant F protein contains an F1 protein that has the sequence set forth in SEQ ID NO:30. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 30.

In some embodiments, the F protein is a mutant NiV-F protein that is a biologically active portion thereof that comprises a 20 amino acid truncation at or near the C-terminus of the wild-type NiV-F protein (SEQ ID NO:28); and a point mutation on an N-linked glycosylation site. In some embodiments, the mutant NiV-F protein comprises an amino acid sequence set forth in SEQ ID NO: 31. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 31.

In some embodiments, the F protein is a mutant NiV-F protein that is a biologically active portion thereof that comprises a 22 amino acid truncation at or near the C-terminus of the wild-type NiV-F protein (SEQ ID NO:28). In some embodiments, the F protein or the biologically active portion is a truncated NiV-F that lacks amino acids 525-546 of SEQ ID NO:3. In some embodiments, the NiV-F protein is encoded by a nucleotide sequence that encodes the sequence set forth in SEQ ID NO: 32. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

In some embodiments, the F protein is a mutant NiV-F protein that is a biologically active portion thereof that comprises a 22 amino acid truncation at or near the C-terminus of the wild-type NiV-F protein (SEQ ID NO:28). In some embodiments, the F protein or the biologically active portion is a truncated NiV-F that lacks amino acids 525-546 of SEQ ID NO:3. In some embodiments, the NiV-F protein is encoded by a nucleotide sequence that encodes the sequence set forth in SEQ ID NO: 33. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 33.

### 2. G Proteins

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof. A non-limited list of exemplary G proteins is shown in Table 2.

The attachment G proteins are type II transmembrane glycoproteins containing an N-terminal cytoplasmic tail (e.g. corresponding to amino acids 1-49 of SEQ ID NO:5), a transmembrane domain (e.g. corresponding to amino acids 50-70 of SEQ ID NO:5, and an extracellular domain containing an extracellular stalk (e.g. corresponding to amino acids 71-187 of SEQ ID NO:5), and a globular head (corresponding to amino acids 188-602 of SEQ ID NO:5). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lipid particle can contain heterologous proteins from different species.

TABLE 2
Exemplary Henipavirus G Proteins

| | | | SEQ ID NO |
|---|---|---|---|
| | | | (without N- |
| Viral G | | SEQ | terminal |
| Protein | Sequence | ID NO | methionine) |
| Hendra Virus G | MMADSKLVSLNNNLSGKIKDQGKVIKNYYGTMDIKK | 6 | 7 |
| Protein | INDGLLDSKILGAFNTVIALLGSIIIIVMNIMIIQNYTRTT | | |
| | DNQALIKESLQSVQQQIKALTDKIGTEIGPKVSLIDTSS | | |
| | TITIPANIGLLGSKISQSTSSINENVNDKCKFTLPPLKIHE | | |
| | CNISCPNPLPFREYRPISQGVSDLVGLPNQICLQKTTSTI | | |
| | LKPRLISYTLPINTREGVCITDPLLAVDNGFFAYSHLEK | | |
| | IGSCTRGIAKQRIIGVGEVLDRGDKVPSMFMTNVWTPP | | |
| | NPSTIHHCSSTYHEDFYYTLCAVSHVGDPILNSTSWTE | | |
| | SLSLIRLAVRPKSDSGDYNQKYIAITKVERGKYDKVM | | |
| | PYGPSGIKQGDTLYFPAVGFLPRTEFQYNDSNCPIIHCK | | |
| | YSKAENCRLSMGVNSKSHYILRSGLLKYNLSLGGDIIL | | |
| | QFIEIADNRLTIGSPSKIYNSLGQPVFYQASYSWDTMIK | | |
| | LGDVDTVDPLRVQWRNNSVISRPGQSQCPRFNVCPEV | | |
| | CWEGTYNDAFLIDRLNWVSAGVYLNSNQTAENPVFA | | |
| | VFKDNEILYQVPLAEDDTNAQKTITDCFLLENVIWCIS | | |
| | LVEIYDTGDSVIRPKLFAVKIPAQCSES | | |
| Nipah Virus G | MPAENKKVRFENTTSDKGKIPSKVIKSYYGTMDIKKIN | 8 | 9 |
| Protein | EGLLDSKILSAFNTVIALLGSIVIIVMNIMIIQNYTRSTD | | |
| | NQAVIKDALQGIQQQIKGLADKIGTEIGPKVSLIDTSSTI | | |
| | TIPANIGLLGSKISQSTASINENVNEKCKFTLPPLKIHEC | | |
| | NISCPNPLPFREYRPQTEGVSNLVGLPNNICLQKTSNQI | | |
| | LKPKLISYTLPVVGQSGTCITDPLLAMDEGYFAYSHLE | | |
| | RIGSCSRGVSKQRIIGVGEVLDRGDEVPSLFMTNVWTP | | |
| | PNPNTVYHCSAVYNNEFYYVLCAVSTVGDPILNSTYW | | |
| | SGSLMMTRLAVKPKSNGGGYNQHQLALRSIEKGRYD | | |
| | KVMPYGPSGIKQGDTLYFPAVGFLVRTEFKYNDSNCPI | | |
| | TKCQYSKPENCRLSMGIRPNSHYILRSGLLKYNLSDGE | | |
| | NPKVVFIEISDQRLSIGSPSKIYDSLGQPVFYQASFSWD | | |
| | TMIKFGDVLTVNPLVVNWRNNTVISRPGQSQCPRFNT | | |
| | CPEICWEGVYNDAFLIDRINWISAGVFLDSNQTAENPV | | |
| | FTVFKDNEILYRAQLASEDTNAQKTITNCFLLKNKIWC | | |
| | ISLVEIYDTGDNVIRPKLFAVKIPEQCT | | |
| Cedar Virus G | MLSQLQKNYLDNSNQQGDKMNNPDKKLSVNFNPLEL | 10 | 11 |
| Protein | DKGQKDLNKSYYVKNKNYNVSNLLNESLHDIKFCIYC | | |
| | IFSLLIIITIINIITISIVITRLKVHEENNGMESPNLQSIQDS | | |
| | LSSLTNMINTEITPRIGILVTATSVTLSSSINYVGTKTNQ | | |
| | LVNELKDYITKSCGFKVPELKLHECNISCADPKISKSA | | |
| | MYSTNAYAELAGPPKIFCKSVSKDPDFRLKQIDYVIPV | | |
| | QQDRSICMNNPLLDISDGFFTYIHYEGINSCKKSDSFKV | | |
| | LLSHGEIVDRGDYRPSLYLLSSHYHPYSMQVINCVPVT | | |
| | CNQSSFVFCHISNNTKTLDNSDYSSDEYYITYFNGIDRP | | |
| | KTKKIPINNMTADNRYIHFTFSGGGGVCLGEEFIIPVTT | | |
| | VINTDVFTHDYCESFNCSVQTGKSLKEICSESLRSPTNS | | |
| | SRYNLNGIMIISQNNMTDFKIQLNGITYNKLSFGSPGRL | | |
| | SKTLGQVLYYQSSMSWDTYLKAGFVEKWKPFTPNW | | |
| | MNNTVISRPNQGNCPRYHKCPEICYGGTYNDIAPLDL | | |
| | GKDMYVSVILDSDQLAENPEITVFNSTTILYKERVSKD | | |
| | ELNTRSTTTSCFLFLDEPWCISVLETNRFNGKSIRPEIYS | | |
| | YKIPKYC | | |
| Bat | MPQKTVEFINMNSPLERGVSTLSDKKTLNQSKITKQG | 12 | 13 |
| Paramyxovirus G | YFGLGSHSERNWKKQKNQNDHYMTVSTMILEILVVL | | |
| Protein, | GIMFNLIVLTMVYYQNDNINQRMAELTSNITVLNLNL | | |
| Eid_hel/GH- | NQLTNKIQREIIPRITLIDTATTITIPSAITYILATLTTRISE | | |
| M74a/GHA/2009 | LLPSINQKCEFKTPTLVLNDCRINCTPPLNPSDGVKMSS | | |
| | LATNLVAHGPSPCRNFSSVPTIYYYRIPGLYNRTALDE | | |
| | RCILNPRLTISSTKFAYVHSEYDKNCTRGFKYYELMTF | | |
| | GEILEGPEKEPRMFSRSFYSPTNAVNYHSCTPIVTVNE | | |
| | GYFLCLECTSSDPLYKANLSNSTFHLVILRHNKDEKIV | | |
| | SMPSFNLSTDQEYVQIIPAEGGGTAESGNLYFPCIGRLL | | |
| | HKRVTHPLCKKSNCSRTDDESCLKSYYNQGSPQHQV | | |
| | VNCLIRIRNAQRDNPTWDVITVDLTNTYPGSRSRIFGSF | | |
| | SKPMLYQSSVSWHTLLQVAEITDLDKYQLDWLDTPYI | | |
| | SRPGGSECPFGNYCPTVCWEGTYNDVYSLTPNNDLFV | | |
| | TVYLKSEQVAENPYFAIFSRDQILKEFPLDAWISSARTT | | |
| | TISCFMFNNEIWCIAALEITRLNDDIIRPIYYSFWLPTDC | | |
| | RTPYPHTGKMTRVPLRSTYNY | | |
| Mojiang virus, | MATNRDNTITSAEVSQEDKVKKYYGVETAEKVADSIS | 14 | 15 |
| Tongguan 1 G | GNKVFILMNTLLILTGAIITITLNITNLTAAKSQQNMLKI | | |
| Protein | IQDDVNAKLEMFVNLDQLVKGEIKPKVSLINTAVSVSI | | |
| | PGQISNLQTKFLQKYVYLEESITKQCTCNPLSGIFPTSG | | |
| | PTYPPTDKPDDDTTDDDKVDTTIKPIEYPKPDGCNRTG | | |
| | DHFTMEPGANFYTVPNLGPASSNSDECYTNPSFSIGSSI | | |
| | YMFSQEIRKTDCTAGEILSIQIVLGRIVDKGQQGPQASP | | |
| | LLVWAVPNPKIINSCAVAAGDEMGWVLCSVTLTAAS | | |
| | GEPIPHMFDGFWLYKLEPDTEVVSYRITGYAYLLDKQ | | |
| | YDSVFIGKGGGIQKGNDLYFQMYGLSRNRQSFKALCE | | |
| | HGSCLGTGGGGYQVLCDRAVMSFGSEESLITNAYLKV | | |
| | NDLASGKPVIIGQTFPPSDSYKGSNGRMYTIGDKYGLY | | |
| | LAPSSWNRYLRFGITPDISVRSTTWLKSQDPIMKILSTC | | |
| | TNTDRDMCPEICNTRGYQDIFPLSEDSEYYTYIGITPNN | | |
| | GGTKNFVAVRDSDGHIASIDILQNYYSITSATISCFMYK | | |
| | DEIWCIAITEGKKQKDNPQRIYAHSYKIRQMCYNMKS | | |
| | ATVTVGNAKNITIRRY | | |

In some embodiments, the G protein has a sequence set forth in any of SEQ ID NOS: 5-15 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 5, 6, 7, 8, 9, 10, 11, 12, 13, 14, or 15. In particular embodiments, the G protein or functionally active variant or biologically active portion is a protein that retains fusogenic activity in conjunction with a Henipavirus F protein, e.g. NiV—F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 and retains fusogenic activity in conjunction with a Henipavirus F protein (e.g., NiV—F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 and retains fusogenic activity in conjunction with a Henipavirus F protein (e.g., NiV—F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:5, SEQ ID NO:8 or SEQ ID NO:9, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:5, SEQ ID NO:8 or SEQ ID NO:9.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:5, SEQ ID NO:8 or SEQ ID NO:9), up to 6 contiguous amino acid residues at or near the N-terminus of the wild-type NiV-G protein (SEQ ID NO:5, SEQ ID NO:8 or SEQ ID NO:9), up to 7 contiguous amino acid residues at or near the N-terminus of the wild-type NiV-G protein (SEQ ID NO:5, SEQ ID NO:8 or SEQ ID NO:9), up to 8 contiguous amino acid residues at or near the N-terminus of the wild-type NiV-G protein (SEQ ID NO:5, SEQ ID NO:8 or SEQ ID NO:9), up to 9 contiguous amino acid residues at or near the N-terminus of the wild-type NiV-G protein (SEQ ID NO:5, SEQ ID NO:8 or SEQ ID NO:9) up to 10 contiguous amino acid residues at or near the N-terminus of the wild-type NiV-G protein (SEQ ID NO:5, SEQ ID NO:8 or SEQ ID NO:9), up to 11 contiguous amino acid residues at or near the N-terminus of the wild-type NiV-G protein (SEQ ID NO:5, SEQ ID NO:8 or SEQ ID NO:9), up to 12 contiguous amino acid residues at or near the N-terminus of the wild-type NiV-G protein SEQ ID NO:9, SEQ ID NO:5, SEQ ID NO:8 or SEQ ID NO:8), up to 13 contiguous amino acid residues at or near the N-terminus of the wild-type NiV-G protein (SEQ ID NO:5, SEQ ID NO:8 or SEQ ID NO:9), up to 14 contiguous amino acid residues at or near the N-terminus of the wild-type NiV-G protein (SEQ ID NO:5, SEQ ID NO:8 or SEQ ID NO:9), up to 15 contiguous amino acid residues at or near the N-terminus of the wild-type NiV-G protein (SEQ ID NO:5, SEQ ID NO:8 or SEQ ID NO:9) up to 16 contiguous amino acid residues at or near the N-terminus of the wild-type NiV-G protein (SEQ ID NO:5, SEQ ID NO:8 or SEQ ID NO:9), up to 17 contiguous amino acid residues at or near the N-terminus of the wild-type NiV-G protein (SEQ ID NO:5, SEQ ID NO:8 or SEQ ID NO:9), up to 18 contiguous amino acid residues at or near the N-terminus of the wild-type NiV-G protein (SEQ ID NO:5, SEQ ID NO:8 or SEQ ID NO:9), up to 19 contiguous amino acid residues at or near the N-terminus of the wild-type NiV-G protein (SEQ ID NO:5, SEQ ID NO:8 or SEQ ID NO:9), up to 20 contiguous amino acid residues at or near the N-terminus of the wild-type NiV-G protein (SEQ ID NO:5, SEQ ID NO:8 or SEQ ID NO:9), up to 21 contiguous amino acid residues at or near the N-terminus of the wild-type NiV-G protein SEQ ID NO:5, SEQ ID NO:8 or SEQ ID NO:9), up to 22 contiguous amino acid residues at or near the N-terminus of the wild-type NiV-G protein (SEQ ID NO:5, SEQ ID NO:8 or SEQ ID NO:9), up to 23 contiguous amino acid residues at or near the N-terminus of the wild-type NiV-G protein (SEQ ID NO:5, SEQ ID NO:8 or SEQ ID NO:9), up to 24 contiguous amino acid residues at or near the N-terminus of the wild-type NiV-G protein (SEQ ID NO:5, SEQ ID NO:8 or SEQ ID NO:9), up to 25 contiguous amino acid residues at or near the N-terminus of the wild-type NiV-G protein (SEQ ID NO:5, SEQ ID NO:8 or SEQ ID NO:9), up to 26 contiguous amino acid residues at or near the N-terminus of the wild-type NiV-G protein (SEQ ID NO:5, SEQ ID NO:8 or SEQ ID NO:9), up to 27 contiguous amino acid residues at or near the N-terminus of the wild-type NiV-G protein (SEQ ID NO:5, SEQ ID NO:8 or SEQ ID NO:9), up to 28 contiguous amino acid residues at or near the N-terminus of the wild-type NiV-G protein (SEQ ID NO:5, SEQ ID NO:8 or SEQ ID NO:9), up to 29 contiguous amino acid residues at or near the N-terminus of the wild-type NiV-G protein (SEQ ID NO:5, SEQ ID NO:8 or SEQ ID NO:9), up to 30 contiguous amino acid residues at or near the N-terminus of the wild-type NiV-G protein (SEQ ID NO:5, SEQ ID NO:8 or SEQ ID NO:9), up to 31 contiguous amino acid residues at or near the N-terminus of the wild-type NiV-G protein (SEQ ID NO:5, SEQ ID NO:8 or SEQ ID NO:9), up to 32 contiguous amino acid residues at or near the N-terminus of the wild-type NiV-G protein (SEQ ID NO:5, SEQ ID NO:8 or SEQ ID NO:9), up to 33 contiguous amino acid residues at or near the N-terminus of the wild-type NiV-G protein (SEQ ID NO:5, SEQ ID NO:8 or SEQ ID NO:9), up to 34 contiguous amino acid residues at or near the N-terminus of the wild-type NiV-G protein (SEQ ID NO:5, SEQ ID NO:8 or SEQ ID NO:9), up to 35 contiguous amino acid residues at or near the N-terminus of the wild-type NiV-G protein (SEQ ID NO:5, SEQ ID NO:8 or SEQ ID NO:9), up to 36 contiguous amino acid residues at or near the N-terminus of the wild-type NiV-G protein (SEQ ID NO:5, SEQ ID NO:8 or SEQ ID NO:9), up to 37 contiguous amino acid residues at or near the N-terminus of the wild-type NiV-G protein (SEQ ID NO:5, SEQ ID NO:8 or SEQ ID NO:9)up to 38 contiguous amino acid residues at or near the N-terminus of the wild-type NiV-G protein (SEQ ID NO:5, SEQ ID NO:8 or SEQ ID NO:9), up to 39 contiguous amino acid residues at or near the N-terminus of the wild-type NiV-G protein (SEQ ID NO:5, SEQ ID NO:8 or SEQ ID NO:9), up to 40 contiguous amino acid residues at or near the N-terminus of the wild-type NiV-G protein (SEQ ID NO:5, SEQ ID NO:8 or SEQ ID NO:9), up to 41 contiguous amino acid residues at or near the N-terminus of the wild-type NiV-G protein (SEQ ID NO:5, SEQ ID NO:8 or SEQ ID NO:9), up to 42 contiguous amino acid residues at or near the N-terminus of the wild-type NiV-G protein (SEQ ID NO:5, SEQ ID NO:8 or SEQ ID NO:9), up to 43 contiguous amino acid residues at or near the N-terminus of the wild-type NiV-G protein (SEQ ID NO:5, SEQ ID NO:8 or SEQ ID NO:9), up to 44 contiguous amino acid residues at or near the N-terminus of the wild-type NiV-G protein (SEQ ID NO:5, SEQ ID NO:8 or SEQ ID NO:9), or up to 45 contiguous amino acid residues at or near the N-terminus of the wild-type NiV-G protein (SEQ ID NO:5, SEQ ID NO:8 or SEQ ID NO:9).

In some embodiments, the NiV-G protein is a biologically active portion that does not contain a cytoplasmic domain. In some embodiments, the NiV-G protein without the cytoplasmic domain is encoded by SEQ ID NO: 39.

In some embodiments, the mutant NiV-G protein comprises a sequence set forth in any of SEQ ID NOS: 29-31, or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 29-31.

In some embodiments, the mutant NiV-G protein has a 5 amino acid truncation at or near the N-terminus of the wild-type NiV-G protein (SEQ ID NO:1, SEQ ID NO: 5, SEQ ID NO: 8, SEQ ID NO:9). In some embodiments, the encoded NiV-G has a truncation up to amino acid residue 5 of SEQ ID NO:8 but lacks the N-terminal methionine. In some embodiments, the encoded NiV-G has a truncation up to amino acid residue 5 but retains the initial methionine. For instance, in some embodiments, the encoded NiV-G has a truncation of amino acids 2-5 of SEQ ID NO:8. In some embodiments, the NiV-G is set forth in SEQ ID NO: 29 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:29.

In some embodiments, the mutant NiV-G protein has a 20 amino acid truncation at or near the N-terminus of the wild-type NiV-G protein (SEQ ID NO: 1, SEQ ID NO: 5, SEQ ID NO: 8, SEQ ID NO:9). In some embodiments, the encoded NiV-G has a truncation up to amino acid residue 20 of SEQ ID NO:8 but lacks the N-terminal methionine. In some embodiments, the encoded NiV-G has a truncation up to amino acid residue 20 amino acids but retains the initial methionine. For instance, in some embodiments, the encoded NiV-G has a truncation of amino acids 2-20 of SEQ ID NO:8. In some embodiments, the mutant NiV-G is set forth in SEQ ID NO: 30 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:30

In some embodiments, the mutant NiV-G protein has a 25 amino acid truncation at or near the N-terminus of the wild-type NiV-G protein (SEQ ID NO: 1, SEQ ID NO: 5, SEQ ID NO: 8, SEQ ID NO:9). In some embodiments, the encoded NiV-G has a truncation up to amino acid residue 25 of SEQ ID NO:8 but lacks the N-terminal methionine. In some embodiments, the encoded NiV-G has a truncation up to amino acid residue 25 but retains the initial methionine. For instance, in some embodiments, the encoded NiV-G has a truncation of amino acids 2-25 of SEQ ID NO:8. In some embodiments, the mutant NiV-G is set forth in SEQ ID NO: 31 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:31.

In some embodiments, the mutant NiV-G protein has a 34 amino acid truncation at or near the N-terminus of the wild-type NiV-G protein (SEQ ID NO: 1, SEQ ID NO: 5, SEQ ID NO: 8, SEQ ID NO:9). In some embodiments, the encoded NiV-G has a truncation up to amino acid residue 34 of SEQ ID NO:8 but lacks the N-terminal methionine. In some embodiments, the encoded NiV-G has a truncation up to amino acid residue 34 but retains the initial methionine. For instance, in some embodiments, the encoded NiV-G has a truncation of amino acids 2-34 of SEQ ID NO:8. In some embodiments, the mutant NiV-G has the sequence set forth in SEQ ID NO: 34 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:34. In some embodiments, the mutant NiV-G protein has a 33 amino acid truncation at or near the N-terminus of the wild-type NiV-G protein (SEQ ID NO: 1, SEQ ID NO: 5, SEQ ID NO: 8, SEQ ID NO:9), such as set forth in SEQ ID NO: 35 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35.

In some embodiments, the G protein is a mutant HeV-G protein that is a biologically active portion of a wild-type HeV-G. In some embodiments, the biologically active portion is an N-terminally truncated fragment.

In some embodiments, the mutant G protein is a mutant HeV-G protein that has the sequence set forth in SEQ ID NO:40 or 41, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40 or 41.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:40 or 41), up to 6 contiguous amino acid residues at or near the N-terminus of the wild-type HeV-G protein (SEQ ID NO:40 or 41), up to 7 contiguous amino acid residues at or near the N-terminus of the wild-type HeV-G protein (SEQ ID NO:40 or 41 or up to 8 contiguous amino acid residues at or near the N-terminus of the wild-type HeV-G protein (SEQ ID NO:40 or 41), up to 9 contiguous amino acid residues at or near the N-terminus of the wild-type HeV-G protein (SEQ ID NO:40 or 41), up to 10 contiguous amino acid residues at or near the N-terminus of the wild-type HeV-G protein (SEQ ID NO:40 or 41), up to 11 contiguous amino acid residues at or near the N-terminus of the wild-type HeV-G protein (SEQ ID NO:40 or 41), up to 12 contiguous amino acid residues at or near the N-terminus of the wild-type HeV-G protein (SEQ ID NO:40 or 41), up to 13 contiguous amino acid residues at or near the N-terminus of the wild-type HeV-G protein (SEQ ID NO: 40 or 41), up to 14 contiguous amino acid residues at or near the N-terminus of the wild-type HeV-G protein (SEQ ID NO: 40 or 41), up to 15 contiguous amino acid residues at or near the N-terminus of the wild-type HeV-G protein (SEQ ID NO: 40 or 41), up to 16 contiguous amino acid residues at or near the N-terminus of the wild-type HeV-G protein (SEQ ID NO: 40 or 41), up to 17 contiguous amino acid residues at or near the N-terminus of the wild-type HeV-G protein (SEQ ID NO: 40 or 41), up to 18 contiguous amino acid residues at or near the N-terminus of the wild-type HeV-G protein (SEQ ID NO: 40 or 41), up to 19 contiguous amino acid residues at or near the N-terminus of the wild-type HeV-G protein (SEQ ID NO: 40 or 41), up to 20 contiguous amino acid residues at or near the N-terminus of the wild-type HeV-G protein (SEQ ID NO: 40 or 41), up to 21 contiguous amino acid residues at or near the N-terminus of the wild-type HeV-G protein (SEQ ID NO: 40 or 41), up to 22 contiguous amino acid residues at or near the N-terminus of the wild-type HeV-G protein (SEQ ID NO: 40 or 41), up to 23 contiguous amino acid residues at or near the N-terminus of the wild-type HeV-G protein (SEQ ID NO: 40 or 41), up to 24 contiguous amino acid residues at or near the N-terminus of the wild-type HeV-G protein (SEQ ID NO: 40 or 41), up to 25 contiguous amino acid residues at or near the N-terminus of the wild-type HeV-G protein (SEQ ID NO: 40 or 41), up to 26 contiguous amino acid residues at or near the N-terminus of the wild-type HeV-G protein (SEQ ID NO: 40 or 41), up to 27 contiguous amino acid residues at or near the N-terminus of the wild-type HeV-G protein (SEQ ID NO: 40 or 41), up to 28 contiguous amino acid residues at or near the N-terminus of the wild-type HeV-G protein (SEQ ID NO: 40 or 41), up to 29 contiguous amino acid residues at or near the N-terminus of the wild-type HeV-G protein (SEQ ID NO: 40 or 41), up to 30 contiguous amino acid residues at or near the N-terminus of the wild-type HeV-G protein (SEQ ID NO: 40 or 41), up to 31 contiguous amino acid residues at or near the N-terminus of the wild-type HeV-G protein (SEQ ID NO: 40 or 41), up to 32 contiguous amino acid residues at or near the N-terminus of the wild-type HeV-G protein (SEQ ID NO: 40 or 41), up to 33 contiguous amino acid residues at or near the N-terminus of the wild-type HeV-G protein (SEQ ID NO: 40 or 41), up to 34 contiguous amino acid residues at or near the N-terminus of the wild-type HeV-G protein (SEQ ID NO: 40 or 41), up to 35 contiguous amino acid residues at or near the N-terminus of the wild-type HeV-G protein (SEQ ID NO: 40 or 41), up to 36 contiguous amino acid residues at or near the N-terminus of the wild-type HeV-G protein (SEQ ID NO: 40 or 41), up to 37 contiguous amino acid residues at or near the N-terminus of the wild-type HeV-G protein (SEQ ID NO: 40 or 41), up to 38 contiguous amino acid residues at or near the N-terminus of the wild-type HeV-G protein (SEQ ID NO: 40 or 41), up to 39 contiguous amino acid residues at or near the N-terminus of the wild-type HeV-G protein (SEQ ID NO: 40 or 41), up to 40 contiguous amino acid residues at or near the N-terminus of the wild-type HeV-G protein (SEQ ID NO: 40 or 41), up to 41 contiguous amino acid residues at or near the N-terminus of the wild-type HeV-G protein (SEQ ID NO: 40 or 41), up to 42 contiguous amino acid residues at or near the N-terminus of the wild-type HeV-G protein (SEQ ID NO: 40 or 41), up to 43 contiguous amino acid residues at or near the N-terminus of the wild-type HeV-G protein (SEQ ID NO: 40 or 41), up to 44 contiguous amino acid residues at or near the N-terminus of the wild-type HeV-G protein (SEQ ID NO: 40 or 41), or up to 45 contiguous amino acid residues at or near the N-terminus of the wild-type HeV-G protein (SEQ ID NO: 40 or 41). In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO: 40 or 41), such as set forth in SEQ ID NO:68 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:42.

In some embodiments, the G protein or the functionally active variant or biologically active portion thereof binds to Ephrin B2 or Ephrin B3. In some aspects, the G protein has the sequence of amino acids set forth in any one of SEQ ID NO:41, SEQ ID NO:40 or SEQ ID NO:8, SEQ ID NO:10, SEQ ID NO: 9, SEQ ID NO:12 or SEQ ID NO:14, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:41, SEQ ID NO:40 or SEQ ID NO:8, SEQ ID NO:10, SEQ ID NO: 9, SEQ ID NO:12 or SEQ ID NO:14, or a functionally active variant or biologically active portion thereof, and retains binding to Ephrin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, and retains binding to Ephrin B2 or B3. Reference to retaining binding to Ephrin B2 or B3 includes binding that is at least or at least about 5% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 10% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 15% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 20% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 25% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion, 30% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 35% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 40% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 45% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 50% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 55% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 60% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 65% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 70% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 75% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 80% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 85% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 90% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, or such as at least or at least about 95% of the level or degree of binding of the corresponding wild-type protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:8, SEQ ID NO:9 or SEQ ID NO:44, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID SEQ ID NO:8, SEQ ID NO:9 or SEQ ID NO:44 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NO:8, SEQ ID NO:9 or SEQ ID NO:44, 10% of the level or degree of binding of the corresponding wild-type NiV-G, such as set forth in SEQ ID NO:8, SEQ ID NO:9 or SEQ ID NO:44, 15% of the level or degree of binding of the corresponding wild-type NiV-G, such as set forth in SEQ ID NO:8, SEQ ID NO:9 or SEQ ID NO:44, 20% of the level or degree of binding of the corresponding wild-type NiV-G, such as set forth in SEQ ID NO:8, SEQ ID NO:9 or SEQ ID NO:44, 25% of the level or degree of binding of the corresponding wild-type NiV-G, such as set forth in SEQ ID NO:8, SEQ ID NO:9 or SEQ ID NO:4444, 30% of the level or degree of binding of the corresponding wild-type NiV-G, such as set forth in SEQ ID NO:8, SEQ ID NO:9 9or SEQ ID NO:44, 35% of the level or degree of binding of the corresponding wild-type NiV-G, such as set forth in SEQ ID NO:8, SEQ ID NO:9 or SEQ ID NO:44, 40% of the level or degree of binding of the corresponding wild-type NiV-G, such as set forth in SEQ ID NO:8, SEQ ID NO:9 or SEQ ID NO:44, 45% of the level or degree of binding of the corresponding wild-type NiV-G, such as set forth in SEQ ID NO:8, SEQ ID NO:9 or SEQ ID NO:4450% of the level or degree of binding of the corresponding wild-type NiV-G, such as set forth in SEQ ID NO:8, SEQ ID NO:9 or SEQ ID NO:44, 55% of the level or degree of binding of the corresponding wild-type NiV-G, such as set forth in SEQ ID NO:8, SEQ ID NO:9 or SEQ ID NO:44, 60% of the level or degree of binding of the corresponding wild-type NiV-G, such as set forth in SEQ ID NO:8, SEQ ID NO:9 or SEQ ID NO:44, 65% of the level or degree of binding of the corresponding wild-type NiV-G, such as set forth in SEQ ID NO:8, SEQ ID NO:9 or SEQ ID NO:44, 70% of the level or degree of binding of the corresponding wild-type NiV-G, such as set forth in SEQ ID NO:8, SEQ ID NO:9 or SEQ ID NO:44, such as at least or at least about 75% of the level or degree of binding of the corresponding wild-type NiV-G, such as set forth in SEQ ID NO:8, SEQ ID NO:9 or SEQ ID NO:44, such as at least or at least about 80% of the level or degree of binding of the corresponding wild-type NIV-G, such as set forth in SEQ ID NO:8, SEQ ID NO:9 or SEQ ID NO:44, such as at least or at least about 85% of the level or degree of binding of the corresponding wild-type NiV-G, such as set forth in SEQ ID NO:8, SEQ ID NO:9 or SEQ ID NO:44, such as at least or at least about 90% of the level or degree of binding of the corresponding wild-type NiV-G, such as set forth in SEQ ID NO:8, SEQ ID NO:9 or SEQ ID NO:44, or such as at least or at least about 95% of the level or degree of binding of the corresponding wild-type NiV-G, such as set forth in SEQ ID NO:8, SEQ ID NO:9 or SEQ ID NO:44.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:40 or 41, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 40 or 41 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO: 40 or 41, 10% of the level or degree of binding of the corresponding wild-type HeV-G, such as set forth in SEQ ID NO: 40 or 41, 15% of the level or degree of binding of the corresponding wild-type HeV-G, such as set forth in SEQ ID NO:66 or 67, 20% of the level or degree of binding of the corresponding wild-type HeV-G, such as set forth in SEQ ID NO: 40 or 41, 25% of the level or degree of binding of the corresponding wild-type HeV-G, such as set forth in SEQ ID NO: 40 or 41, 30% of the level or degree of binding of the corresponding wild-type HeV-G, such as set forth in SEQ ID NO: 40 or 41, 35% of the level or degree of binding of the corresponding wild-type HeV-G, such as set forth in SEQ ID NO: 40 or 41, 40% of the level or degree of binding of the corresponding wild-type HeV-G, such as set forth in SEQ ID NO: 40 or 41, 45% of the level or degree of binding of the corresponding wild-type HeV-G, such as set forth in SEQ ID NO: 40 or 41, 50% of the level or degree of binding of the corresponding wild-type HeV-G, such as set forth in SEQ ID NO: 40 or 41, 55% of the level or degree of binding of the corresponding wild-type HeV-G, such as set forth in SEQ ID NO: 40 or 41, 60% of the level or degree of binding of the corresponding wild-type HeV-G, such as set forth in SEQ ID NO: 40 or 41, 65% of the level or degree of binding of the corresponding wild-type HeV-G, such as set forth in SEQ ID NO: 40 or 41, 70% of the level or degree of binding of the corresponding wild-type HeV-G, such as set forth in SEQ ID NO:18 or 52, such as at least or at least about 75% of the level or degree of binding of the corresponding wild-type HeV-G, such as set forth in SEQ ID NO: 40 or 41, such as at least or at least about 80% of the level or degree of binding of the corresponding wild-type NIV-G, such as set forth in SEQ ID NO: 40 or 41, such as at least or at least about 85% of the level or degree of binding of the corresponding wild-type HeV-G, such as set forth in SEQ ID NO: 40 or 41, such as at least or at least about 90% of the level or degree of binding of the corresponding wild-type HeV-G, such as set forth in SEQ ID NO: 40 or 41, or such as at least or at least about 95% of the level or degree of binding of the corresponding wild-type HeV-G, such as set forth in SEQ ID NO:40 or 41.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:38.

In some embodiments, the G protein is a mutant G protein containing one or more amino acid substitutions selected from the group consisting of E501A, W504A, Q530A and E533A with reference to numbering set forth in SEQ ID NO:8. In some embodiments, the G protein is a mutant G protein that contains one or more amino acid substitutions elected from the group consisting of E501A, W504A, Q530A and E533A with reference to SEQ ID NO:8and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO: 8), 6 contiguous amino acid residues at or near the N-terminus of the wild-type NiV-G protein (SEQ ID NO: 8), 7 contiguous amino acid residues at or near the N-terminus of the wild-type NiV-G protein (SEQ ID NO: 8), 8 contiguous amino acid residues at or near the N-terminus of the wild-type NiV-G protein (SEQ ID NO: 8), 9 contiguous amino acid residues at or near the N-terminus of the wild-type NiV-G protein (SEQ ID NO: 8), up to 10 contiguous amino acid residues at or near the N-terminus of the wild-type NiV-G protein (SEQ ID NO: 8), 11 contiguous amino acid residues at or near the N-terminus of the wild-type NiV-G protein (SEQ ID NO: 8), 12 contiguous amino acid residues at or near the N-terminus of the wild-type NiV-G protein (SEQ ID NO: 8), 13 contiguous amino acid residues at or near the N-terminus of the wild-type NiV-G protein (SEQ ID NO: 8), 14 contiguous amino acid residues at or near the N-terminus of the wild-type NiV-G protein (SEQ ID NO: 8), up to 15 contiguous amino acid residues at or near the N-terminus of the wild-type NiV-G protein (SEQ ID NO: 8), 16 contiguous amino acid residues at or near the N-terminus of the wild-type NiV-G protein (SEQ ID NO: 8), 17 contiguous amino acid residues at or near the N-terminus of the wild-type NiV-G protein (SEQ ID NO: 8), 18 contiguous amino acid residues at or near the N-terminus of the wild-type NiV-G protein (SEQ ID NO: 8), 19 contiguous amino acid residues at or near the N-terminus of the wild-type NiV-G protein (SEQ ID NO: 8), up to 20 contiguous amino acid residues at or near the N-terminus of the wild-type NiV-G protein (SEQ ID NO: 8), 21 contiguous amino acid residues at or near the N-terminus of the wild-type NiV-G protein (SEQ ID NO: 8) 22 contiguous amino acid residues at or near the N-terminus of the wild-type NiV-G protein (SEQ ID NO: 8), 23 contiguous amino acid residues at or near the N-terminus of the wild-type NiV-G protein (SEQ ID NO: 8), 24 contiguous amino acid residues at or near the N-terminus of the wild-type NiV-G protein (SEQ ID NO: 8), up to 25 contiguous amino acid residues at or near the N-terminus of the wild-type NiV-G protein (SEQ ID NO: 8), 26 contiguous amino acid residues at or near the N-terminus of the wild-type NiV-G protein (SEQ ID NO: 8), 27 contiguous amino acid residues at or near the N-terminus of the wild-type NiV-G protein (SEQ ID NO: 8), 28 contiguous amino acid residues at or near the N-terminus of the wild-type NiV-G protein (SEQ ID NO: 8), 29 contiguous amino acid residues at or near the N-terminus of the wild-type NiV-G protein (SEQ ID NO: 8), up to 30 contiguous amino acid residues at or near the N-terminus of the wild-type NiV-G protein (EQ ID NO: 8), up to 31 contiguous amino acid residues at or near the N-terminus of the wild-type NiV-G protein (SEQ ID NO: 8), 32 contiguous amino acid residues at or near the N-terminus of the wild-type NiV-G protein (SEQ ID NO: 8), 33 contiguous amino acid residues at or near the N-terminus of the wild-type NiV-G protein (SEQ ID NO: 8) 34 contiguous amino acid residues at or near the N-terminus of the wild-type NiV-G protein (SEQ ID NO: 8), 35 contiguous amino acid residues at or near the N-terminus of the wild-type NiV-G protein (SEQ ID NO: 8) up to 36 contiguous amino acid residues at or near the N-terminus of the wild-type NiV-G protein (EQ ID NO: 8), up to 37 contiguous amino acid residues at or near the N-terminus of the wild-type NiV-G protein (EQ ID NO: 8), up to 38 contiguous amino acid residues at or near the N-terminus of the wild-type NiV-G protein (EQ ID NO: 8), up to 39 contiguous amino acid residues at or near the N-terminus of the wild-type NiV-G protein (SEQ ID NO: 8), or up to 40 contiguous amino acid residues at or near the N-terminus of the wild-type NiV-G protein (EQ ID NO: 8).

In some embodiments, the mutant NiV-G protein has the amino acid sequence set forth in SEQ ID NO: 34 or 35 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 34 or 35. In particular embodiments, the G protein has the sequence of amino acids set forth in SEQ ID NO 34 or 35.

In some embodiments, the mutant NiV-G protein has the amino acid sequence set forth in SEQ ID NO:34 and the mutant NiV-F protein has the amino acid sequence set forth in SEQ ID NO:32. In some embodiments, the mutant NiV-G protein has the amino acid sequence set forth in SEQ ID NO:35 and the mutant NiV-F protein has the amino acid sequence set forth in SEQ ID NO:32.

In some embodiments, the G protein is a mutant G protein containing one or more amino acid substitutions selected from the group consisting of E501A, W504A, Q530A and E533A with reference to numbering set forth in SEQ ID NO:10. In some embodiments, the G protein is a mutant G protein that contains one or more amino acid substitutions elected from the group consisting of E501A, W504A, Q530A and E533A with reference to SEQ ID NO:10 and is a biologically active portion thereof containing an N-terminal truncation.

a. Retargeting Moieties

In some embodiments, the G protein, e.g. NiV-G, is a targeted envelope protein that contains a targeting moiety. In some embodiments, the targeting moiety binds a target ligand. In some embodiments, the target ligand can be expressed in an organ or cell type of interest, e.g., the lung. In particular embodiments, the G protein, such as NiV-G, is mutated to reduce binding for the native binding partner of the G protein. In some embodiments, the G protein is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a G protein, such as NiV-G, can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred.

In some embodiments, the G protein (e.g. NiV-G) may be re-targeted by covalently conjugating a targeting-moiety to the fusion protein. In some embodiments, the G protein (e.g. NiV-G) and targeting moiety are covalently conjugated by expression of a chimeric protein comprising the G protein linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to G proteins to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to G proteins to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to G proteins to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, G proteins may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered G proteins may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi- specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s.

In embodiments, the re-targeted G protein binds a cell surface marker on the target cell, e.g., a protein, glycoprotein, receptor, cell surface ligand, agonist, lipid, sugar, class I transmembrane protein, class II transmembrane protein, or class III transmembrane protein.

In some embodiments, lipid membrane bound particle-surface targeting moiety is a peptide. In some embodiments, vector-surface targeting moiety is an antibody, such as a single domain antibody. In some embodiments, the antibody can be human or humanized. In some embodiments, antibody or portion thereof is naturally occurring. In some embodiments, the antibody or portion thereof is synthetic.

In some embodiments, the antibody can be generated from phage display libraries to have specificity for a desired target ligand. In some embodiments the target ligand is expressed in the lung, such as ACE2. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the lipid membrane bound particle-surface targeting moiety is attached to the C-terminus of the G protein or biologically active portion thereof. In some embodiments, the N-terminus of the vector-surface targeting moiety is exposed on the exterior surface of the lipid bilayer. In some embodiments, the N-terminus of the vector-surface targeting moiety binds to a cell surface molecule of a target cell. In some embodiments, the lipid membrane bound particle-surface targeting moiety specifically binds to a cell surface molecule present on a target cell. In some embodiments, the vector-surface targeting moiety is a protein, glycan, lipid or low molecular weight molecule.

In some embodiments, the cell surface ligand of a target cell is an antigen or portion thereof. In some embodiments, the vector-surface targeting moiety or portion thereof is an antibody having a single monomeric domain antigen binding/recognition domain that is able to bind selectively to a specific antigen. In some embodiments, the single domain antibody binds an antigen present on a target cell.

Exemplary cells include lung stem cells, bronchiolar epithelial cells, alveolar epithelial cells, stromal cells, type 1 and II pneumocytes also known as alveolar type I and II epithelial cells, basal cells, secretory cells, club cells, clara cells, ciliated cells, capillary cells, alveolar macrophages, and lung epithelial cells. In some embodiments, the target cell is an epithelial cell. In some embodiments, the ligand is expressed on a host cell, such as an epithelial cell.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments, the target tissue is the lung.

In some embodiments, the C-terminus of the vector-surface targeting moiety is attached to the C-terminus of the G protein or biologically active portion thereof. In some embodiments, the N-terminus of the vector-surface targeting moiety is exposed on the exterior surface of the lipid bilayer. In some embodiments, the N-terminus of the vector-surface targeting moiety binds to a cell surface molecule of a target cell. In some embodiments, the vector-surface targeting moiety specifically binds to a cell surface molecule present on a target cell. In some embodiments, the vector-surface targeting moiety is a protein, glycan, lipid or low molecular weight molecule.

In some embodiments, the cell surface marker is a molecule expressed on a target cell that is an antigen or portion thereof recognized by the targeting moiety.

Exemplary target cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cardiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+cancer cell, a CD19+ cancer cell, a Her2/Neu+cancer cell, a GluA2+ neuron, a GluA4+ neuron, a NKG2D+natural killer cell, a SLC1A3+ astrocyte, a SLC7A10+ adipocyte, or a CD30+ lung epithelial cell.

In some embodiments, the targeting moiety binds to a target antigen on a T cell. In some embodiments, the target antigen is CD4. In some embodiments, the target antigen is CD8. The targeting moiety may be any binding domain able to bind to the target antigen, e.g. CD4 or CD8. For instance, the targeting moiety can be an antibody or antigen-binding fragment, such as an scFv or a single domain antibody (e.g. VHH). Various antibodies and antigen-binding fragments thereof for targeting a T cell antigen, such as CD4 or CD8, are known.

Exemplary targeting moieties for binding to CD8 (CD8 binding agents) include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Ser. No. 10/730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SKi, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,587,022, 9,745,552; as well as antibodies B486A1, RPA-T4, CE9.1 (Novus Biologicals); GK1.5, RM4-5, RPA-T4, OKT4, 4SM95, S3.5, N1UGO (ThermoFisher); GTX50984, ST0488, 10BS, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds.

In some embodiments, the target cell is an antigen presenting cell, an MHC class II+cell, a professional antigen presenting cell, an atypical antigen presenting cell, a macrophage, a dendritic cell, a myeloid dendritic cell, a plasmacyteoid dendritic cell, a CD11c+cell, a CD11b+cell, a splenocyte, a B cell, a hepatocyte, a endothelial cell, or a non-cancerous cell).

In some embodiments, lipid membrane bound particles may display targeting moieties that are not conjugated to G proteins in order to redirect the fusion activity towards a cell that is bound by the targeting moiety, or to affect homing.

In some embodiments, the targeting moiety added to the lipid membrane bound particle is modulated to have different binding strengths. In some embodiments, scFvs and antibodies with various binding strengths may be used to alter the fusion activity of the vector vehicle particle towards cells that display high or low amounts of the target antigen (doi:10.1128/JVI.01415-07, doi:10.1038/cgt.2014.25, DOI: 10.1002/jgm.1151). In some embodiments, DARPins with different affinities may be used to alter the fusion activity of the vector vehicle particle towards cells that display high or low amounts of the target antigen (doi:10.1038/mt.2010.298). In some embodiments, targeting moieties may also be modulated to target different regions on the target ligand, which will affect the fusion rate with cells displaying the target (doi: 10.1093/protein/gzv005).

### B. Viral Vector Particles and Methods For Producing Same

In some embodiments, the lipid particles are viral-based particles and the host cells contain or are introduced with one or more nucleic acid sequences derived from a virus, in which culture of the cells can result in production of the viral-based particles. In some embodiments, the lipid membrane bound particle produced by the provided methods is a viral vector, such as a vector particle derived from lentivirus.

The lipid membrane bound particles produced by the provided methods include lentiviral vectors, including those that can be used to transduce cells in connection with cell or gene therapy. Methods of producing a lentivirus are known. Exemplary methods are described in, e.g., Wang et al., J. Vis Exp. (32):1499, 2009; Merten et al., Mol Ther Methods Clin Dev (3): 16017, 2016, and Salmon et al., Curr Protoc Hum Genet (12): 12, 2007.

Current methods of producing a lentivirus are in most aspects related to the transient transfection of cells. More recent developments include suspension culture processes, and the implementation of stable producer cell lines. In some aspects, downstream processing results in lipid membrane bound particles, i.e., lentiviral vector, which can be used in methods of viral based vector gene delivery.

In some embodiments, the methods include use of viral nucleic acids, and in some cases one or more transgene or exogenous payload gene, provided on plasmids. In some embodiments, the plasmids include transfer vectors and packaging vectors for producing viral vectors, such as lentiviral vectors. The viral vector genome is typically constructed in a plasmid form that can be transfected into a packaging or producer cell line. Any of a variety of known methods can be used to produce retroviral particles, such as lentiviral particles, whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components. Exemplary features of transfer and packaging plasmids for contained or introduced into host cells for producing viral vectors, such as lentiviral vectors, are described in subsections below.

In some embodiments, the method include use of plasmids for lentiviral replication. In some embodiments, the assembly of a lipid membrane bound particle produced by the provided methods, such as a viral vector, is optionally initiated by binding of the core protein to a unique encapsidation sequence within the viral genome (e.g. UTR with stem-loop structure). In some embodiments, the interaction of the core with the encapsidation sequence facilitates oligomerization.

In some embodiments, the viral vector particle produced by the provided methods is replication deficient. In some embodiments, the vector particle is integration deficient. A number of preclinical studies have demonstrated therapeutic and prophylactic efficacy of viral based vector gene delivery in animal models and in clinical trials. In some aspects, viral and virally derived vectors capable of replication provide consistent gene expression over time. In some aspects, replication competent viruses can result in undesired immunogenicity, toxicity, and cell death. In some embodiments, vectors capable of insertion are efficient for transduction of a variety of cells. However, in some aspects, they can pose a risk of insertional mutagenesis. Integration-deficient vectors can persist episomally but can also retain the transduction efficiency of standard integrating vectors. Various methods of rendering a vector insertional or replication deficient are known in the art.

In some embodiments, the lipid membrane particle produced by the provided methods is a virus-like particle (VLP) derived from a virus, such as lentivirus. The VLPs include those derived from retroviruses or lentiviruses. While VLPs mimic native virion structure, they lack the viral genomic information necessary for independent replication within a host cell. Therefore, in some aspects, VLPs are non-infectious. In some embodiments, the lipid membrane bound particle's bilayer of amphipathic lipids is or comprises lipids derived from a cell. A VLP typically comprises at least one type of structural protein from a virus. In most cases this protein will form a proteinaceous capsid (e.g. VLPs comprising a lentivirus, adenovirus or paramyxovirus structural protein). In some cases the capsid will also be enveloped in a lipid bilayer originating from the cell from which the assembled VLP has been released (e.g. VLPs comprising a human immunodeficiency virus structural protein such as GAG). In some embodiments, the VLP's bilayer of amphipathic lipids is or comprises the viral envelope. In some embodiments, the VLP produced by the provided method further comprises a targeting moiety as an envelope protein within the lipid bilayer.

In some embodiments, the lipid membrane bound particle is a lipid particle which comprises a sequence that is devoid of or lacking viral RNA, which in some aspects may be the result of removing or eliminating the viral RNA from the sequence. In some embodiments, this may be achieved by using an endogenous packaging signal binding site on gag. In some embodiments, the endogenous packaging signal binding site is on pol. In some embodiments, the polynucleotides provided herein, will contain a cognate packaging signal. In some embodiments, a heterologous binding domain (which is heterologous to gag) located on the polynucleotides provided herein to be delivered, and a cognate binding site located on gag or pol, can be used to ensure packaging of any of the heterologous genes or polynucleotides provided herein to be delivered. In some embodiments, the lipid membrane bound particles could be used to deliver the polynucleotides or polypeptides provided herein, in which case functional integrase and/or reverse transcriptase is not required.

In some embodiments, the lipid membrane bound particle produced by the provided method comprises supramolecular complexes formed by viral proteins that self-assemble into capsids. In some embodiments, the lipid membrane bound particle produced by the provided method is a virus-like particle derived from viral capsid proteins. In some embodiments, the lipid membrane bound particle is a virus-like particle derived from viral nucleocapsid proteins. In some embodiments, the lipid membrane bound particle comprises nucleocapsid-derived proteins that retain the property of packaging nucleic acids. In some embodiments, the viral-based lipid membrane bound particle produced by the provided method, such as virus-like particles comprises only viral structural glycoproteins. In some embodiments, lipid membrane bound particle does not contain a viral genome. 1. Transfer Plasmids

In some embodiments, the provided viral vectors contain a genome derived from a retroviral genome based vector, such as derived from a gammaretroviral or lentiviral genome based vector. For instance, in provided aspects, the viral vectors contain a nucleic acid that includes virus-derived nucleic acid elements that typically facilitate transfer of the nucleic acid molecule or integration into the genome of a cell or to a viral particle that mediates nucleic acid transfer. Typically, this is provided by a transfer plasmid (sometimes also referred to as a transfer vector) that contains the sequences that will incorporate into the host cell genome but cannot produce functional viral particles without the genes encoded in the envelope and packaging vectors. The transfer plasmid typically also contains a gene of interest such as a gene encoding an exogenous agent as described herein. In some aspects, vector particles will typically include various viral components and sometimes also host cell components in addition to nucleic acid(s). In some embodiments, a lipid membrane bound particle that is a viral vector is capable of transferring a nucleic acid into a cell.

Any of a large number of such suitable vector genomes are known ((see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557; Pfeifer and Verma (2001) Annu. Rev. Genomics Hum. Genet., 2:177-211).

In some embodiments, the transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral lipid membrane bound particle) may optionally comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, in the viral vector particles described herein at least part of one or more protein coding regions that contribute to or are essential for replication may be absent compared to the corresponding wild-type virus. In some embodiments, the viral vector is replication-defective. In some embodiments, the viral vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques. Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV).

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1 and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral lipid membrane bound vector particle comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit viral based vector to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV—I and HIV-2. In addition this method could be used to increase expression of genes from HTLV-I, HTLV-2, HFV, HSRV and human endogenous retroviruses (HERV), MLV and other retroviruses.

In embodiments, the retroviral vector particle comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector particle, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector particle is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector particle described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Packaging Plasmids

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acid encoding an exogenous agent or payload gene, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an exogenous agent; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

Large scale vector particle production is often useful to achieve a desired concentration of vector particles. Vector vehicle particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a producer cell, and are introduced into the cell via transfection, transduction or infection. A retroviral, e.g., lentiviral, transfer vector can be introduced into a producer cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines (also called packaging cell line or packaging cells) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, a producer source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the producer source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, the producer or packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

### C. Plasmids for the Expression of an Exogenous Agent

In some embodiments, the lipid membrane bound particle as described contains an exogenous agent. In some embodiments, the lipid membrane bound described herein contains a nucleic acid that encodes an exogenous agent. In some embodiments, the lipid membrane bound particle contains the exogenous agent. In some embodiments, the lipid membrane bound particle contains a nucleic acid that encodes an exogenous agent. Reference to the coding sequence of the nucleic acid encoding the exogenous agent also is referred to herein as a payload gene or heterologous gene. In some embodiments, the exogenous agent or the nucleic acid encoding the exogenous agent are present in the lumen of the lipid membrane bound particle. In some embodiments, a nucleic acid encoding the exogenous agent is incorporated into the transfer plasmid for production of the lipid particle (e.g. viral vectors, such as lentiviral vector).

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule. In some embodiments, the lipid membrane bound particle is a particle which further comprises an encapsulated polypeptide or polynucleotide encoding a heterologous gene, a therapeutic gene, an exogenous gene, and/or a recombinant gene, such as any recombinant gene, particularly a therapeutic gene.

In some embodiments, the exogenous agent is loaded into the lipid membrane bound particle via electroporation into the lipid membrane bound particle itself or into the cell from which the lipid membrane bound particle is derived. In some embodiments, the exogenous agent is loaded into the lipid membrane bound particle via transfection (e.g., of a DNA such as a heterologous gene or an mRNA encoding the exogenous agent) into the lipid membrane bound particle itself or into the cell from which the lipid membrane bound particle is derived.

In some embodiments, the exogenous agent may include one or more nucleic acid sequences, one or more polypeptides, a combination of nucleic acid sequences and/or polypeptides, one or more organelles, and any combination thereof.

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells.

In some embodiments, a lipid membrane bound particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide.

In some embodiments, the lipid membrane bound particle contains a nucleic acid that encodes a protein exogenous agent (also referred to as a “heterologous, recombinant, exogenous, or therapeutic gene.”). In some embodiments, the lipid membrane bound particle is a particle which further comprises an exogenous agent that is an encapsulated polypeptide.

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell.

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the exogenous agent is a nuclease for use in gene editing methods. In some embodiments, the nuclease is a zinc-finger nucleases (ZFNs), transcription-activator like effector nucleases (TALENs), or a CRISPR-associated protein- nuclease (Cas). In some embodiments, the Cas is Cas9 from Streptococcus pyogenes. In some embodiments, the Cas is a Cas12a (also known as cpf1) from a Prevotella or Francisella bacteria, or the Cas is a Cas12b from a Bacillus, optionally Bacillus hisashii. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020)

In some embodiments, the provided the lipid membrane bound particle contains a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, the provided lipid membrane bound particle comprises one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid membrane bound particle (e.g. paramyxovirus lipid particles). For instance, a chimeric Cas9-protein fusion with the structural GAG protein can be packaged inside a paramyxovirus lipid particle. In some embodiments, the fusion protein is a cleavable fusion protein between (i) a viral structural protein (e.g. GAG) and (ii) a nuclease protein (e.g. Cas protein, such as Cas 9).

In some embodiments, the lipid membrane bound particle is a particle which further comprises an encapsulated polypeptide or polynucleotide encoding a heterologous gene, a therapeutic gene, an exogenous gene, and/or a recombinant gene, such as any recombinant gene, particularly a therapeutic gene.

In some embodiments, the exogenous agent comprises a nucleic acid (i.e., a heterologous, recombinant, exogenous, or therapeutic gene) that encodes a cytosolic protein, e.g., a protein that is produced in the recipient cell and localizes to the recipient cell cytoplasm. In some embodiments, the exogenous agent comprises a nucleic acid that encodes a secreted protein, e.g., a protein that is produced and secreted by the recipient cell. In some embodiments, the exogenous agent comprises a nucleic acid that encodes a nuclear protein, e.g., a protein that is produced in the recipient cell and is imported to the nucleus of the recipient cell. In some embodiments, the exogenous agent comprises a nucleic acid that encodes an organellar protein (e.g., a mitochondrial protein), e.g., a protein that is produced in the recipient cell and is imported into an organelle (e.g., a mitochondrial) of the recipient cell.

In some embodiments, the exogenous agent comprises a nucleic acid (i.e., a heterologous, recombinant, exogenous, or therapeutic gene) that encodes a membrane protein. In some embodiments, the membrane protein comprises a nucleic acid that encodes a chimeric antigen receptor (CAR), a T cell receptor, an integrin, an ion channel, a pore forming protein, a Toll-Like Receptor, an interleukin receptor, a cell adhesion protein, or a transport protein. In some embodiments, delivery of the nuclease is by a provided vector encoding the nuclease (e.g. Cas).

For example, the heterologous, recombinant, exogenous, or therapeutic gene can be, but is not limited to antisense ras, antisense myc, antisense raf, antisense erb, antisense src, antisense fins, antisense jun, antisense trk, antisense ret, antisense gsp, antisense hst, antisense bc1, antisense ab1, Rb, CFTR, pi 6, p21, p27, p57, p73, C-CAM, APC, CTS-I, zac1, scFV ras, DCC, NF-I, NF-2, WT-I, MEN-I, MEN-II, BRCA1, VHL, MMACl, FCC, MCC, BRCA2, IL-I, IL-2, IL-3, IL-4, IL-5, IL-6, IL-7, IL-8, IL-9, IL-10, IL-11 IL-12, GM-CSF, G-CSF, thymidine kinase, mda7, fus-1, interferon α, interferon β, interferon γ, ADP, p53, ABLI, BLCl, BLC6, CBFAl, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIMI, PML, RET, SRC, TALI, TCL3, YES, MADH4, RB1, TP53, WT1, TNF, BDNF, CNTF, NGF, IGF, GMF, aFGF, bFGF, NT3, NT5, ApoAI, ApoAIV, ApoE, RaplA, cytosine deaminase, Fab, ScFv, BRCA2, zac1, ATM, HIC-I, DPC-4, FHIT, PTEN, ING1, NOEY1, NOEY2, OVCAl, MADR2, 53BP2, IRF-I, Rb, zac1, DBCCR-I, rks-3, COX-I, TFPI, PGS, Dp, E2F, ras, myc, neu, raf, erb, fins, trk, ret, gsp, hst, abl, ElA, p300, VEGF, FGF, thrombospondin, BAI-I, GDAIF, or MCC. In further embodiments of the present invention, the recombinant gene is a gene encoding an ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyrne, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. In other embodiments of the present invention, the recombinant gene is a gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, gmcose-6-phosphatase, low-density-lipoprotein receptor, porphobilinogen deaminase, factor VIII, factor IX, cystathione α-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase. galactose- 1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase. Alternatively, the recombinant gene may encode growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

### D. Culture of Permissive Host Cells and Conditions for Cultivation

In some embodiments, the provided methods include culturing host cost cells comprising one or more nucleic acids for the production of the lipid membrane bound particles as described. In some embodiments, the host cells are mammalian cells. Mammalian cells for the production of a membrane bound lipid particle, such as any of the provided virus-like particles, a lentiviruses, viral vectors, or lentiviral vectors, viral based particles, or VLPs, are known in the art. Examples of contemplated cells include Human Embryonic Kidney (HEK) 293 cells and derivatives thereof (for example the 293SF-3F6 line). HEK are capable of growth in suspension under serum-free conditions and are considered highly transfectable. Other cell types include, but are not limited to, HeLa (Henrietta Lacks) cells, A549 cells, KB cells, CKT1 cells, NIH/sT3 cells, Vera cells, Chinese Hamster Ovary (CHO) cells, or any mammalian and/or eukaryotic cell which is permissive to lentiviral infection or can support the lentiviral lifecycle.

In a some embodiments, the cells are 293T cells, which are commercially available from a number of suppliers. This cell line is derived from human embryonic kidney cells transformed with SV40 large-T antigen and requiring serum supplementation, often fetal bovine, for growth. Specifically, the HEK 293 cell line was originally derived from human embryonic kidney cells transfected with fragments of sheared human adenovirus type 5 (Ad5) and subsequent selection of cells that showed transformation or characteristic thereof. The transforming region of human adenovirus contains early region 1 (E1), consisting of two transcription units, E1a and E1b, whose products are necessary and sufficient for mammalian cell transformation by Ads. These 293 cells are a subclone of the original Frank Graham 293 cells which were selected for higher virus yield (likely using adenovirus) and better cell growth (Graham et al, 1977, J Gen Virol, 36, 59-74). From HEK 293 cells, the 293T cell line was created in the laboratory of Michele Calos (Stanford University) by transfection with a gene encoding the SV-40 T-antigen and a neomycin resistance gene. Adherent 293 T cells have also been used for producing lentiviral vectors.

In some embodiments, the cells are cultured in a serum-free medium, which is selected with respect to the specific cell used. The serum- free medium allows production of lentiviral lipid particle suitable for therapeutic applications. In general, serum free media can be manipulated to enhance growth of the respective cell line in culture, with a potential for supplementation with any of the following: a selection of secreted cellular proteins, diffusible nutrients, amino acids, organic and/or inorganic salts, vitamins, trace metals, sugars, and lipids as well as other compounds such as growth promoting substances (e.g., cytokines). In some embodiments, such media is supplemented with glutamine or an alternative to glutamine such as GlutaMAX™, as disclosed herein. Such media are commercially available, and the person skilled in the art will be able to select the appropriate ones with respect to the mammalian and/or eukaryotic host cells. In some embodiments, the medium may be supplemented with additives such as a non- ionic surfactant used for controlling shear forces in suspension cultures, an anti-clumping agent and L-glutamine or an alternative to L-glutamine such as a L-alanyl-L- glutamine dipeptide, e.g. GlutaMAX™ (Invitrogen, catalogue No 35050-038). The media and additives used in the present invention are advantageously GMP compliant. For example, representative commercially available serum- free media which can be used for growing 293T cells in suspension include F17 medium® (Invitrogen) and Ex-Cell 293® (SAFC). In particular, 293T cells can be grown in customized F17 medium® supplemented with additives preventing the formation of cell aggregates.

In some embodiments, the cells, e.g., the host cells are cultivated in a volume of media that is, is about, or is at least 100 mL, 200 mL, 300 mL, 400 mL, 500 mL, 600 mL, 700 mL, 800 mL, 900 mL, 1,000 mL, 2,000 mL, 3,000 mL, 4,000 mL, 5,000 mL. In some embodiments, the host cells are cultivated in a volume of media that is, is about, or is at least 6,000 mL, 7,000 mL, 8,000 mL, 9,000 mL, or 10,000 mL. In some embodiments, the host cells are cultivated in a volume of media that is, is about, or is at least 11,000 mL, 12,000 mL, 13,000 mL, 14,000 mL, 15,000 mL, 16,000 mL, 17,000 mL, 18,000 mL, 19,000 mL, 20,000 mL. In some embodiments, the host cells are cultivated in a volume of media that is, is about, or is at least 25,000 mL, 30,000 mL, 35,000 mL, 40,000 mL, 45,000 mL, 50,000 mL, 55,000 mL, 60,000 mL, 65,000 mL, 70,000 mL, 75,000 mL, 80,000 mL, 90,000 mL, 95,000 mL, 100,000 mL. In some embodiments, the host cells are cultivated in a volume of media that is, is about, or is at least, 125,000 mL, 150,000 mL, 175,000 mL, 200,000 mL.

In some embodiments, the cells are cultivated at an initial volume that is later adjusted to a different volume. In particular embodiments, the volume is later adjusted during the cultivation. In particular embodiments, the volume is increased from the initial volume during the cultivation. In certain embodiments, the volume is increased when the cells achieve a density during the cultivation. In certain embodiment, the initial volume is or is about 5,000 mL.

After culture is initiated in the culture medium, parameters such as pH, temperature, and level of dissolved oxygen are controlled to the prescribed levels during, or during at least a portion of, the cell culture. Exemplary methods for controlling or maintaining culture parameters are described below.

In some embodiments, the cultivation is performed under conditions that generally include a temperature suitable for the growth of primary human cells, for example, at least about 25 degrees Celsius, generally at least about 30 degrees, and generally at or about 37 degrees Celsius. In some embodiments, the composition of host cells is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, concentration, number or dose of cells. In some embodiments, the incubation is carried out for a time period until the culture, e.g. cultivation or expansion, results in a desired or threshold density, concentration, number or dose of viable cells. In some embodiments, the incubation is greater than or greater than about or is for about or 24 hours, 48 hours, 72 hours, 96 hours, 5 days, 6 days, 7 days, 14 days, 21 days or more. In some embodiments, density, concentration and/or number or dose of the cells can be determined or monitored during the cultivation, such as by using methods as described, including optical methods, including digital holography microscopy (DHM) or differential digital holography microscopy (DDHM).

### 1. Potential of Hydrogen (pH)

In some aspects, provided embodiments relate to findings that the pH set point of a cell culture process can affect the cell-culture performance (e.g., including in the production of lipid particles such as the paramyxovirus lipid particles described herein). The wide fluctuations in pH that often occur during the process of cell culture as, for example, when medium is replenished or cells reach the exponential growth phase, can have an effect on the cells and their ability to efficiently produce paramyxovirus lipid particles in high titers. In provided embodiments, the provided methods maintain or control the pH during at least a portion of the culturing of host cells for the production of the lipid particles to improve the efficient production and functional titer of the produced lipid particles, such as pseudotyped lentiviral vectors.

In some embodiments, pH can be shifted (e.g., maintained) by an active and/or passive approach as described in more detail below.

In some embodiments, an “active shift” in the pH is a change in the setpoint of the pH to a new value. In some embodiments, active shift is induced by adding any respective pH changing and regulating agent(s) known to those skilled in the art. In some embodiments, “passive shift” with respect to pH indicates that during a passive shift in pH the cells themselves are allowed to change the pH of the medium by accumulation of metabolic products, thus following a cell culture specific metabolic pH profile within a predefined pH range. In some embodiments, this is accomplished by defining one pH setpoint and a deadband (±) wherein the pH is allowed to change. In contrast to the active shift, the passive shift or change in pH is not induced by adding the respective pH changing agent(s).

In some embodiments, pH regulating agents are added to the cultures in order to maintain the pH at a specific setpoint or to change the pH during a pH shift. Typical pH regulating agents used for cell culturing purposes include liquid base or acid solutions such as NaOH or HCl.

In some embodiments, the cells are cultivated at a pH that is maintained during culturing of the cells. In some embodiments, the pH of the culture is at or about 6.7 to at or about 7.7. In some embodiments, the pH of the culture is at or about 6.8 to at or about 7.5. In some embodiments, the pH of the culture is at or about 6.9 to at or about 7.4. In some embodiments, the pH of the culture is at or about 7.0 to at or about 7.4. In some embodiments, the pH of the culture is at or about 7.1 to at or about 7.3.

In some embodiments, the methods include culturing in which the pH is maintained at or about pH 6.7, at or about pH 6.8, at or about pH 6.9, at or about pH 7.0, at or about pH 7.1, at or about pH 7.2, at or about pH 7.3, at or about pH 7.4, at or about pH 7.5, or at or about pH 7.6, at or about pH 7.7 or any value between any of the foregoing. In some embodiments, it is understood that maintaining the pH at any of the above-described pH does not mean that the pH is kept constant during the entire culturing as some fluctuation of pH may occur that is then adjusted during the culturing the maintain the pH. Typically the pH is within a prescribed range of any of the above pH values, such as a range that is ±0.05, ±0.10, ±0.20, or ±0.50.

In some embodiments, a pH setpoint and dead band is selected. In some embodiments, the pH set point is at or about pH 6.7, at or about pH 6.8, at or about pH 6.9, at or about pH 7.0, at or about pH 7.1, at or about pH 7.2, at or about pH 7.3, at or about pH 7.4, at or about pH 7.5, or at or about pH 7.6, at or about pH 7.7 or any value between any of the foregoing. In some embodiments, the pH set point is a set point at a pH between 7.0 and 7.4. In some embodiments, the pH set point is at or about pH 7.0, at or about pH 7.05, at or about pH 7.10, at or about pH 7.15, at or about pH 7.2, at or about pH 7.25, at or about pH 7.3, at or about pH 7.35 or at or about pH 7.40. In some embodiments, the pH is at or about pH 7.05. In some embodiments, the pH is at or about pH 7.1. In some embodiments, the pH is at or about 7.15. In some embodiments, the pH is at or about 7.20. In some embodiments, the pH 8s at or about 7.35.

In some embodiments, the pH is slightly basic. In some embodiments, the pH is at or about 7.1 to at or about 7.3.

In some embodiments, the pH is 7.05. In some embodiments, the pH is 7.20. In some embodiments, the pH is 7.35. In some embodiments, the pH is 7.15.

In some embodiments, the pH is allowed to change from the set point within a deadband that ranges from at or about 0.05 to at or about 0.50, such as from 0.05 to 0.20. In some embodiments, the pH is allowed to change at a pH set point in a deadband from 0.05, 0.10, 0.15, 0.20, 0.25, 0.30, 0.35, 0.40, 0.45 or 0.50 or any value between any of the foregoing. In some of any of the above embodiments, the pH dead band is 0.05 (i.e. ±0.05). In some embodiments, the pH dead band is at or about pH 0.10 (i.e. ±0.10). In some embodiments, the pH dead band is at or about pH 0.15 (i.e. ±0.15). In some embodiments, the pH dead band is at or about pH 0.20 (i.e. ±0.20). In some embodiments, the pH dead band is at or about pH 0.30 (i.e. ±0.30). In some embodiments, the pH dead band is at or about pH 0.40 (i.e. ±0.40). In some embodiments, the pH dead band is at or about pH 0.50 (i.e. ±0.50).

In some embodiments, the pH set point is a set point at a pH between 7.0 and 7.4 and with a deadband value that is a value from 0.05 to 0.20. In some embodiments, the pH set point is at or about pH 7.0 with a deadband of ±0.05 to ±0.20, at or about pH 7.05 with a deadband of ±0.05 to ±0.20, at or about pH 7.10 with a deadband of ±0.05 to ±0.20, at or about pH 7.15 with a deadband of ±0.05 to ±0.20, at or about pH 7.2 with a deadband of ±0.05 to ±0.20, at or about pH 7.25 with a deadband of ±0.05 to ±0.20, at or about pH 7.3 with a deadband of ±0.05 to ±0.20, at or about pH 7.35 with a deadband of ±0.05 to ±0.20 or at or about pH 7.40 with a deadband of ±0.05 to ±0.20. In some embodiments, the pH is at or about pH 7.05 with a deadband of ±0.05. In some embodiments, the pH is at or about pH 7.1 with a deadband of ±0.05. In some embodiments, the pH is at or about 7.15 with a deadband of ±0.05. In some embodiments, the pH is at or about 7.20 with a deadband of ±0.05. In some embodiments, the pH is at or about 7.35 with a deadband of ±0.05. In some embodiments, the pH is at or about pH 7.05 with a deadband of ±0.10. In some embodiments, the pH is at or about pH 7.1 with a deadband of ±0.10. In some embodiments, the pH is at or about 7.15 with a deadband of ±0.10. In some embodiments, the pH is at or about 7.20 with a deadband of ±0.10. In some embodiments, the pH is at or about 7.35 with a deadband of ±0.10. In some embodiments, the pH is at or about pH 7.05 with a deadband of ±0.15. In some embodiments, the pH is at or about pH 7.1 with a deadband of ±0.15. In some embodiments, the pH is at or about 7.15 with a deadband of ±0.15. In some embodiments, the pH is at or about 7.20 with a deadband of ±0.15. In some embodiments, the pH is at or about 7.35 with a deadband of ±0.15. In some embodiments, the pH is at or about pH 7.05 with a deadband of ±0.20. In some embodiments, the pH is at or about pH 7.1 with a deadband of ±0.20. In some embodiments, the pH is at or about 7.15 with a deadband of ±0.20. In some embodiments, the pH is at or about 7.20 with a deadband of ±0.20. In some embodiments, the pH is at or about 7.35 with a deadband of ±0.20.

In some embodiments, the pH during culturing is maintained at 7.1±0.50. In some embodiments, the pH during culturing is maintained at pH is 7.1±0.20. In some embodiments, the pH during culturing is maintained at pH is 7.1±0.10. In some embodiments, the pH during culturing is maintained at pH is 7.1±0.05. In some embodiments, the pH during culturing is maintained at pH is 7.2±0.50. In some embodiments, the pH during culturing is maintained at pH is 7.2±0.20. In some embodiments, the pH during culturing is maintained at pH is 7.2±0.10. In some embodiments, the pH during culturing is maintained at pH is 7.2±0.05. In some embodiments, the pH during culturing is maintained at pH is 7.1±0.50. In some embodiments, the pH during culturing is maintained at pH is 7.1±0.20. In some embodiments, the pH during culturing is maintained at pH is 7.1±0.10. In some embodiments, the pH during culturing is maintained at pH is 7.1±0.05. In some embodiments, the pH is at or about 7.1, 7.2, 7.3.

In some embodiments, the media pH is monitored during the cultivation step. In some embodiments, the monitoring is performed manually, such as by a human operator. In some embodiments, the monitoring is performed by an automated system. In some embodiments, the monitoring is performed by a module. The automated system may require minimal or no manual input to monitor the pH of media for cultivated cells. In some embodiments, the monitoring is performed both manually and by an automated system.

In certain embodiments, the pH is monitored by an automated system, such as system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the media pH conditions are monitored by a module compatible with an automated system or bioreactor, such as an automated module.

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every hour for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 12 hours for the duration of the cultivating step. In some embodiments, the monitoring is performed at least once a day for the duration of the cultivating step. In some embodiments, the monitoring is performed at least once during the cultivating step.

In some embodiments, the cells are cultivated at a pH that is maintained. In some embodiments, the pH is at or about 6.7-7.7. In some embodiments, the pH is mildly basic. In some embodiments, the pH is 7.1-7.3. In some embodiments, the pH is at or about 7.0, at or about 7.1, at or about 7.2, or at or about 7.3.

In some embodiments, the media pH is maintained during the cultivation step. In some embodiments, the maintaince is performed manually, such as by a human operator. In some embodiments, the maintaince is performed by an automated system. In some embodiments, the maintaince is performed by a module. The automated system may require minimal or no manual input to maintain the pH of media for cultivated cells. In some embodiments, the maintaince is performed both manually and by an automated system.

In certain embodiments, the pH is maintained by an automated system, such as system requiring no manual input. In some embodiments, the automated system is compatible with a bioreactor, for example a bioreactor as described herein, such that media can be maintained within the bioreactor. In some embodiments, the media pH conditions are maintained by a module compatible with an automated system or bioreactor, such as an automated module.

After culture is initiated in the culture medium, the pH of the culture may be maintained or controlled to the prescribed levels during, or during at least a portion of, the cell culture. pH is typically controlled by adding basic or acidic solutions when necessary during the process. Commonly used base solutions include sodium bicarbonate, sodium carbonate and sodium hydroxide solutions. In some embodiments, a buffer may be incorporated into the medium to maintain a set pH. In some embodiments maintenance of pH may be achieved by the combination of a) controlling the flow of biogas allowed to exit the bioreactor b) controlling the flow of gas that is allowed to enter the bioreactor; and c) addition of carbonate, bicarbonate, and/or hydroxide of alkali or alkali earth metals including Na, Ca, and Mg to the bioreactor.

In some embodiments, carbon dioxide (CO2) is often necessary to maintain pH because it is constantly being stripped by the air/oxygen sparge. In some embodiments, dissolution of carbon dioxide (CO2) is commonly used to achieve a more acidic pH. In some embodiments, to increase pH a base may be added, such as a carbonate. In some embodiments, the pH control strategy is achieved by using carbon dioxide to lower pH (make more acidic) and sodium carbonate to increase pH (more basic). In some aspects, bicarbonate buffer is used. In other embodiments, the dissolved CO2 and sodium bicarbonate combination forms can be used as a buffer system for the cell culture. In some embodiments, the pH in the culture is thereafter maintained with further additions of bicarbonate or carbon dioxide. For example, lactic acid generation by the cell culture process may prompt further bicarbonate addition until a pH of 7.05-7.7 (i.e., 7.1-7.3) is attained when the bicarbonate partially decomposes into carbon dioxide.

In some embodiments, pH is maintained by adding sodium hydroxide as required to maintain pH within the desired range. In some embodiments, the use of sodium hydroxide may avoid further increase in bicarbonate and an associated increase in dissolved carbon dioxide.

In some embodiments, maintaining the pH in the culture of permissive host cells comprises providing an initial minimum amount of bicarbonate to adjust the pH of the cell culture medium to fall within the desired pH range (i.e., 7.05-7.7). In some embodiments, pH is maintained by adding sodium hydroxide as required to maintain pH within the desired range (i.e., 7.05-7.7) to avoid further increase in bicarbonate and an associated increase in dissolved carbon dioxide. In some embodiments, sodium hydroxide is used to also maintain pH within the desired range.

Other means of maintaining a set pH with a buffer include sodium carbonate and CO₂. For example, in some embodiments changes in pH can be controlled by adding HCO₃ or increasing the carbon dioxide tension. In some embodiments, cell culture pH can also be controlled when replenishing with fresh medium.

### 2. Dissolved Oxygen (DO)

Oxygen is necessary for cell respiration and cell division, and is therefore added to cell culture to maximize cell growth and lipid membrane bound particle production. Dissolving oxygen into liquid cell culture medium is impacted by a number of factors, including temperature and pH, all of which must be considered when making measurements of dissolved oxygen (DO) in liquid, such as liquid media for the culture of permissive cells as described herein.

In some embodiments, the DO is monitored during the cultivation step. In some embodiments, the monitoring is performed manually, such as by a human operator. In some embodiments, the monitoring is performed by an automated system. In some embodiments, the monitoring is performed by a module. The automated system may require minimal or no manual input to monitor DO of media for cultivated cells. In some embodiments, the monitoring is performed both manually and by an automated system.

In certain embodiments, DO is monitored by an automated system, such as system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the media DO conditions are monitored by a module compatible with an automated system or bioreactor, such as an automated module.

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every hour for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 12 hours for the duration of the cultivating step. In some embodiments, the monitoring is performed at least once a day for the duration of the cultivating step. In some embodiments, the monitoring is performed at least once during the cultivating step.

In some embodiments, the cells are cultivated at a dissolved oxygen that is maintained. In some embodiments, the DO is at or about 20%, 25%, 30%, 35%, 40%, 45%, 50%, 55%, 60%, 65%, or any value between any of the foregoing. In some embodiments, the DO is at or about between 40% to 60%, inclusive. In some embodiments, the DO is 20%. In some embodiments, the DO is 25%. In some embodiments, the DO is 30%. In some embodiments, the DO is 35%. In some embodiments, the DO is 40%. In some embodiments, the DO is 45%. In some embodiments, the DO is 50%. In some embodiments, the DO is 55%. In some embodiments, the DO is 60%. In some embodiments, the DO is 65%.

In some embodiments, the methods include culturing in which the DO is maintained at or about 30%, at or about 35%, at or about 40%, at or about 45%, at or about 50%, at or about 55% or at or about 60% or any value between any of the foregoing. In some embodiments, the DO is maintained at 25%. In some embodiments, the DO is maintained at 30%. In some embodiments, the DO is maintained at 35%. In some embodiments, the DO is maintained at 40%. In some embodiments, the DO is maintained at 45%. In some embodiments, the DO is maintained at 50%. In some embodiments, the DO is maintained at 55%. In some embodiments, the DO is maintained at 60%. In some embodiments, the DO is maintained at 65%.

In some embodiments, it is understood that maintaining the DO at any of the above-described DO does not mean that the DO is kept constant during the entire culturing as some fluctuation of DO may occur that is then adjusted during the culturing the maintain the DO. Typically the pH is within a prescribed range of any of the above DO values, such as a range that is ±0.10%, ±1.0%, or ±10%.

In some embodiments, the media DO is maintained during the cultivation step. In some embodiments, the maintaince is performed manually, such as by a human operator. In some embodiments, the maintaince is performed by an automated system. In some embodiments, the maintaince is performed by a module. The automated system may require minimal or no manual input to maintain the DO of media for cultivated cells. In some embodiments, the maintaince is performed both manually and by an automated system.

In certain embodiments, the DO is maintained by an automated system, such as system requiring no manual input. In some embodiments, the automated system is compatible with a bioreactor, for example a bioreactor as described herein, such that media can be maintained within the bioreactor. In some embodiments, the media DO conditions are maintained by a module compatible with an automated system or bioreactor, such as an automated module.

In some embodiments, the desired level of dissolved oxygen in the culture medium or solution is achieved through air sparging, such as to remove or strip dissolved carbon dioxide from a mammalian cell culture solution. In some embodiments, dissolved oxygen can be controlled by sparging the cell culture solution with air or a gas mixture of air/oxygen/nitrogen. In some embodiments, the desired level of dissolved oxygen in the cell culture is achieved using sparger installed on the bottom of the bioreactor, along with agitation of the culture medium or solution using impellers which breakup the large air/oxygen bubbles to enhance the transfer of oxygen to the cell medium from the sparged air bubbles. In some embodiments, sparging is achieved in agitated tanks. In some embodiments, the desired level of dissolved oxygen in the culture medium or solution is achieved by surface gas exchange.

In some embodiments, the DO is maintained via a bioreactor system having an upward flow impeller disposed within a draft tube disposed in the bioreactor vessel. The upward pumping impeller is driven via shaft by a motor outside the bioreactor vessel. The upward flow of the impeller provides a top surface renewal method that enhances surface gas exchange in a highly controllable manner. The upward pumping impeller moves cell culture medium and suspended mammalian cells from the bottom of the bioreactor vessel toward the liquid/headspace gas interface in the upper part of the reactor. In doing so, dissolved carbon dioxide in the cell culture solution or medium is continuously and rapidly brought to the surface of the liquid in the bioreactor where gas-liquid exchange is occurring. A high turnover in the surface liquid allows rapid removal of dissolved carbon dioxide to the headspace. The upward flow impeller allows a higher pumping velocity without creating sufficient shear to damage or kill the mammalian cells. A sweeping gas composed of oxygen, nitrogen, air, carbon dioxide or other suitable gases and mixtures thereof that is introduced to the headspace in the bioreactor vessel, where it interacts with the top surface of the solution to achieve the desired liquid gas exchange, and is subsequently exhausted from the headspace in the bioreactor vessel. Such a system is described in WO 2010/017338.

In some embodiments, carbon dioxide is added at least at the start of the culture of permissive cells to adjust the medium pH to a set value (i.e., between 30 and 60% saturation). In some embodiments, additional carbon dioxide is used to maintain culture grown in lower volume.

In some aspects, cells in the exponential growth phase become maximally metabolically active (i.e., each cell produces its maximum carbon dioxide output). In some embodiments, carbon dioxide can be removed by sparging the broth with air or sweeping the headspace of the bioreactor with a cover gas or air. Examples of carbon dioxide stripping systems and methods are disclosed in United States provisional patent application serial number WO 2010/0173377 and in WO 2010/017338.

In some embodiments, the cells are cultured according to the methods above. In some embodiments, the pH is 7.05, the deadband is 0.05 (±0.05), and the DO is 20%. In some embodiments, the pH is 7.05, the deadband is 0.05 (±0.05), and the DO is 25%. In some embodiments, the pH is 7.05, the deadband is 0.05 (±0.05), and the DO is 30%. In some embodiments, the pH is 7.05, the deadband is 0.05 (±0.05), and the DO is 35%. In some embodiments, the pH is 7.05, the deadband is 0.05 (±0.05), and the DO is 40%. In some embodiments, the pH is 7.05, the deadband is 0.05 (±0.05), and the DO is 45%. In some embodiments, the pH is 7.05, the deadband is 0.05 (±0.05), and the DO is 50%. In some embodiments, the pH is 7.05, the deadband is 0.05 (±0.05), and the DO is 55%. In some embodiments, the pH is 7.05, the deadband is 0.05 (±0.05), and the DO is 60%.

In some embodiments, the pH is 7.20, the deadband is 0.05 (±0.05), and the DO is 20%. In some embodiments, the pH is 7.20, the deadband is 0.05 (±0.05), and the DO is 25%. In some embodiments, the pH is 7.20, the deadband is 0.05 (±0.05), and the DO is 30%. In some embodiments, the pH is 7.20, the deadband is 0.05 (±0.05), and the DO is 35%. In some embodiments, the pH is 7.20, the deadband is 0.05 (±0.05), and the DO is 40%. In some embodiments, the pH is 7.20, the deadband is 0.05 (±0.05), and the DO is 45%. In some embodiments, the pH is 7.20, the deadband is 0.05 (±0.05), and the DO is 50%. In some embodiments, the pH is 7.20, the deadband is 0.05 (±0.05), and the DO is 55%. In some embodiments, the pH is 7.20, the deadband is 0.05 (±0.05), and the DO is 60%.

In some embodiments, the pH is 7.35, the deadband is 0.05 (±0.05), and the DO is 20%. In some embodiments, the pH is 7.35, the deadband is 0.05 (±0.05), and the DO is 25%. In some embodiments, the pH is 7.35, the deadband is 0.05 (±0.05), and the DO is 30%. In some embodiments, the pH is 7.35, the deadband is 0.05 (±0.05), and the DO is 35%. In some embodiments, the pH is 7.35, the deadband is 0.05 (±0.05), and the DO is 40%. In some embodiments, the pH is 7.35, the deadband is 0.05 (±0.05), and the DO is 45%. In some embodiments, the pH is 7.35, the deadband is 0.05 (±0.05), and the DO is 50%. In some embodiments, the pH is 7.35, the deadband is 0.05 (±0.05), and the DO is 55%. In some embodiments, the pH is 7.35, the deadband is 0.05 (±0.05), and the DO is 60%.

In some embodiments, the cells are cultured according to the methods above. In some embodiments, the pH is 7.15, the deadband is 0.15 (±0.15), and the DO is 20%. In some embodiments, the pH is 7.15, the deadband is 0.15 (±0.15), and the DO is 25%. In some embodiments, the pH is 7.15, the deadband is 0.15 (±0.15), and the DO is 30%. In some embodiments, the pH is 7.15, the deadband is 0.15 (±0.15), and the DO is 35%. In some embodiments, the pH is 7.15, the deadband is 0.15 (±0.15), and the DO is 40%. In some embodiments, the pH is 7.15, the deadband is 0.15 (±0.15), and the DO is 45%. In some embodiments, the pH is 7.15, the deadband is 0.15 (±0.15), and the DO is 50%. In some embodiments, the pH is 7.15, the deadband is 0.15 (±0.15), and the DO is 55%. In some embodiments, the pH is 7.15, the deadband is 0.15 (±0.15), and the DO is 60%.

In some embodiments, the cells are cultured according to the methods above. In some embodiments, the pH is 7.15, the deadband is 0.10 (±0.10), and the DO is 20%. In some embodiments, the pH is 7.15, the deadband is 0.10 (±0.10), and the DO is 25%. In some embodiments, the pH is 7.15, the deadband is 0.10 (±0.10), and the DO is 30%. In some embodiments, the pH is 7.15, the deadband is 0.10 (±0.10), and the DO is 35%. In some embodiments, the pH is 7.15, the deadband is 0.10 (±0.10), and the DO is 40%. In some embodiments, the pH is 7.15, the deadband is 0.10 (±0.10), and the DO is 45%. In some embodiments, the pH is 7.15, the deadband is 0.10 (±0.10), and the DO is 50%. In some embodiments, the pH is 7.15, the deadband is 0.10 (±0.10), and the DO is 55%. In some embodiments, the pH is 7.15, the deadband is 0.10 (±0.10), and the DO is 60%.

E. Harvest and/or Purification of Viral Particles

Following culture of permissive cells as described in Sections I.A-C and disclosed in any of the methods herein, the lipid membrane bound particles may then be harvested (or collected), with one or more harvesting step. In some embodiments, the provided method further comprises a single harvest of the particles present in the cell culture. In some embodiments, the method further comprises several collections of the culture, optionally with medium changes. In some embodiments, the method comprises a single harvest, optionally without changing the culture medium from seeding into the bioreactor to the harvest. In some embodiments, a single harvest is carried out 48 hours post-transfection. The lipid membrane bound particles thus produced by any of the methods disclosed herein can then be harvested and purified according to methods also well known by the skilled artisan.

In some embodiments, transformed permissive host cells are harvested and lysed to release the lipid membrane bound particles, such as viral vector particles, from the cells in the form of crude cell lysate. The produced crude cell lysate (CCL) is then purified by double CsCl gradient ultracentrifugation. The cells may be harvested on day 1, 2, 3, 4, 5, 6 post infection. In some embodiments, the permissive host cells are harvested following infection but prior to lysis. Lysis includes, but is not limited to freeze-thaw, autolysis, or detergent lysis methods. In certain aspects cell lysis is by detergent lysis.

In some embodiments, the method further comprises lysing the host cells to provide a cell lysate comprising vector particles using hypotonic solution, a hypertonic solution, an impinging jet, microfludization, solid shear, a detergent, liquid shear, high pressure extrusion, autolysis, sonication methods, or any combination thereof. Suitable detergents include those commercially available as Thesit®., NP-40®, Tween-20®, Brij-58®, Triton X-100® and octyl glucoside. According to one aspect of the invention the detergent is present in the lysis solution at a concentration of at least about, at most about, or about 0.5, I 3 1.5, or 2% (w/v).

The concentration of contaminating nucleic acids in the CLL can be decreased by treating a lysate with a nuclease such as those available commercially as Benzonase® or Pulmozym® In some embodiments, cells are lysed within a tissue culture apparatus, for example a CellCube™. In some embodiments, the cells are lysed after being removed from the tissue culture apparatus. In particular embodiments, the cells are lysed and harvested using detergent(s). In other embodiments, lysis is achieved through autolysis of infected or transformed cells.

Any method of isolating the membrane bound particles from the culture preparation known to those of skill in the art is included by the present disclosure. Aspects of the method include purifying membrane bound lipid particles, such as lentiviral vector particles, from the lysate by one or more of size partitioning purification, tangential flow filtration, column chromatography, including ion exchange chromatography, such as anion exchange chromatography, or any combination thereof. In some embodiments, a size partitioning membrane is in a tangential flow filtration device. In some embodiments, the size partitioning membrane is a dialysis membrane, a porous filter, or is in a tangential flow filtration device. The filtration rate can be a circulating speed of at least about, at most about, or about 500, 750, 1000 to 1000, 1250, 1500 mL/min/fsf2 and the filtration pressure is within the range of at least about, at most about, or about 0, 1, 5, 10 to 10, 20, 30 psig, or any value or range there between. In certain aspects the filtration pressure is at least about, at most about, or about 10 psig. The size of the partitioning pores can be selected on the basis of the size of the virus to be retained, in which case a membrane having a pore or inclusion size sufficiently smaller than the virus to retain the virus and permit the passage of contaminants is selected. Similarly, if the pore or inclusion size is too small, some undesirable contaminants may be retained. Therefore, an optimal pore size is one that retains the most virus yet permits the passage of the most contaminants. In some aspects, the pore size is at most about, or about, 0.10 μm, 0.08 μm, 0.05 μm, 0.03 μm, or 0.01 μm, or any range or value there between. In some embodiments, the size partitioning purification could be carried out by gel filtration purification.

The methods may also include concentrating and diafilitering the lysate. Diafiltration can be by tangential flow filtration. In a further aspect the concentration fold may be in the range of at least about, at most about, or about 5-fold, 10-fold, 15-fold to 20-fold, or more, including any value or range there between. The feeding flow rate may be in the range of at least about, at most about, or about 500, 600, 700, 800, 900, 1000, 1100, 1200, 0.1300, 1400 or 1500 ml/min, or any range or value there between. Typically the purified lipid membrane bound particle has a purity of less than 10, 5, 1, 0.5, or 0.1 nanograms of contaminating DNA per 1 milliliter dose. In certain embodiments, a composition will comprise at least about, at most about, or about 1×10, 5×10¹², 1×10¹³, 5×10¹³, 1×10¹⁴, 5×10¹⁴, 1×10¹⁵, 5×10¹⁵, 1×10¹⁶, 5×10¹⁶ or 1×10¹⁷ lipid particles, including all values there between. In some embodiments, the membrane bound lipid particles are obtained from a single culture preparation. In some embodiments, the methods comprise a concentration step employing membrane filtration. Membrane filtration may utilize a 100 to IOOOK NMWC. regenerated cellulose, or polyether sulfone membrane.

Some embodiments of the present invention involve analysis of virus production. For example, virus production may be analyzed using HPLC. Any technique for analyzing virus production known to those of skill is contemplated by the present invention.

### 1. Titer Determination

In some embodiments, the provided methods produce pseudotyped vectors at high titer, such as compared to alternative methods in which the pH or the DO are not controlled or maintained as described herein. For instance, improved functional titer production of NiV pseudotyped vectors is shown to be achieved herein under slightly basic conditions, such as between pH 7.1 and 7.3. In addition, cell culture medium with particular dissolved oxygen concentration, such as between 40 and 60 percent air saturation, as shown herein also can impact NiV vector production.

In some embodiments, the provided methods result in a ratio of infectious titer to physical titer. In some embodiments, this is expressed as a ratio of transducing units (TU) to the concentration of p24 (i.e., as determined via ELISA as described above). In some embodiments, the ratio of infectious titer to physical titer of the lipid membrane bound particles or lentiviral vector particles is at or about or lower than 10,000: 1, 5,000:1, 2,500:1, 1,000:1, or 100:1. In some embodiments, the ratio of infectious titer to physical titer of the lipid membrane bound particles (e.g. viral vector, such as lentiviral vector particles) is between 10,000:1 and 100:1, 10,000:1 and 1,1000:1, 10:000:′ and 2,500:1, 1:10,000 and 5000:1, 5,000:1 and 100:1, 5,000:1 and 1000:1, 5000:1 and 2500:1, 2500:1 and 100:1, 2500:1 and 1000:1, or 1000:1 and 100:1. In some embodiments, the ratio of infectious titer to physical titer of the lipid membrane bound particles (e.g. viral vector, such as lentiviral vector particles) is between 10,000:1 and 1000:1. In some embodiments, the ratio of infectious titer to physical titer of the lipid membrane bound particles (e.g. viral vector, such as lentiviral vector particles) is between 10,000:1 and 5000:1.

In some embodiments, the provided methods can be conducted in scalable stirred tank bioreactors. Thus, the provided methods can be adapted for scale to provide an optimized means to produce large quantities (i.e., high titers) of NiV pseudotyped viral vectors. The provided embodiments provide for a simple and scalable method to produce Nipah Virus pseudotyped lentiviral vectors with enhanced quality (infectivity) and quantity (functional titer) compared to traditional methods.

In some embodiments, an “infectious” virus or viral particle is one that comprises a polynucleotide component which it is capable of delivering into a cell for which the viral species is trophic (i.e., natural affinity via cognate receptor expression). The term does not necessarily imply any replication capacity of the virus. For example, a replication deficient vector can deliver a polynucleotide and is therefore considered “infectious”. In some aspects, methods for determining infectious titer are known in the art.

For example, infectious titer can be determined via an assay using cells that express a cellular receptor to which the viral protein can bind. In an exemplary high-throughput titering assay, an array of culture wells each comprising an aliquot of mammalian cells and an aliquot of viral vector preparation is established. Control conditions comprising cells alone, vector alone and media are also used. The array of culture wells may, for example, be in the form of a microtiter vessel. Typically, aliquots (i.e., serially diluted aliquots) of the vector preparation to be titered are added to the cells, and then the cells and vector virus are incubated under conditions that allow for replication of the virus (i.e., culture conditions as described in Section I.D). Following a period of infection, PFUs (plaque forming units) can be determined by quantification of the number of “plaques” (i.e., foci of cell death within the culture monolayer), wherein the number of plaques under consideration of the dilution and viral concentration provide information about the infectious titer (i.e., PFU/mL).

Alternatively, cell transduction assays can be used to determine infectious titer in transducing units (TU) for those viruses which may have a less pronounced cytopathic effect in a traditional plaque assay format. In some aspects, a fluorophore can be used to tag the vector sequence such that successfully transduced cells can be quantified via flow cytometry.

In some aspects, the specific infectivity of viral preparations is defined by the ratio of physical viral particles to infectious viral particles

Titer of a produced lentiviral vector particle can also be expressed as a function of physical titer. In some aspects, the physical titer is defined as the concentration of viral particles containing viral genomes. Methods for determining physical titer include methods of quantification of viral genomes, including by use of specific primers in qPCR or other similar DNA based methods (including digital droplet PCR, for example). In some aspects, determination of physical titer can include quantification of a viral protein (i.e., a capsid protein). For example, physical titer can be determined by assaying for the presence of p24 via any known method of protein quantification. In some aspects, physical titer is determined via ELISA for p24 and then scaling for the preparation by multiplying by a known number of vector particles per unit of p24. In some embodiments, physical titer is determined via ELISA for p24 and then multiplying the measured pg/mL of p24 by 12,500.

### II. Cell Culture System

In some embodiments, the methods as described are performed in a cell culture system. In some embodiments, the cultivation of permissive host cells is performed in a closed system, such as a closed cell culture system. In certain embodiments, the cultivation is performed in a closed system under sterile conditions.

The operating conditions of the cell culture may be monitored or measured by any technique known to those of skill in the art, e.g., monitoring the pH of the media and dissolved oxygen tension of the media as is disclosed in Section I.A.C. Growth medium can be inoculated to an initial population of host cells of at least about, at most about, or about 1×10⁴ cells/ml to about 1×10⁶ cells/ml, including any value or range of values there between. In another aspect the initial population of host cells are at a concentration of at least about, at most about, or about 1×10⁴, 5×10⁴, 1×10⁵, 5×10⁵, or 1×10⁶ cells/ml, or any value or range there between.

The host cells may be grown at least part of the time in a perfusion chamber, a bioreactor, a flexible bed platform, or by fed batch. The cells may be grown as a cell suspension culture or alternatively as an anchorage-dependent culture. In other embodiments, media used during any of the growth, inoculating, harvesting, and/or production phases does not contain protein and/or animal-derived products. Alternatively, host cells may be stable in serum-free and/or protein-free media.

Any bioreactor known to those of skill in the art that is capable of supporting host cell growth is contemplated for use. Any size of bioreactor is contemplated by the present invention, e.g., a bioreactor may be at least about, at most about, or about 1 L, 5 L, 10 L, 20 L up to 200 L or larger bioreactor, all volumes inclusive of any value there between. In some embodiments a bioreactor is a bag, such as a cell bag bioreactor having a volume of at least about, at most about, or about 1, 5, 10, 20, 50, 100, 500 to 1000 L cell bag or any volume there between.

In some embodiments, a bioreactor used for cell culture is typically sterile or is sterilized. In some embodiments, the bioreactor is equipped with various probes as well as connections for supplemental gas supply and introduction of additional feeds. Temperature probes, pH detectors, dissolved oxygen probes and dissolved CO2 probes or sensors are used to monitor the temperature, pH, dissolved oxygen and dissolved CO2 levels of the cell medium or solution in real time. In addition, cell culture medium or solution samples can be withdrawn from the bioreactor at selected intervals to determine cell density and cell viability, as well as to analyze other characteristics such as metabolites and osmolality. Based on such analytical results, additional feed or other additives can be added to the cell culture medium or solution in an effort to prolong the cell viability and increase production of biological products.

In some embodiments, at least a portion of the culture is carried out with mixing. In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. A bioreactor can comprise a bioreactor that uses axial rocking of a planar platform to induce wave motions inside of the bioreactor. In some embodiments, wave motions are induced inside of a sterilized polyethylene bag wherein the host cells are located. In further embodiments, the bioreactor is a disposable bioreactor. The bioreactor may be a commercially-available bioreactor, e.g., a Wave Bioreactor® (Wave Biotech, LLC, Bedminster, NJ).

In certain embodiments, at least a portion of the cultivation of permissive host cells is performed with perfusion. In some embodiments, the at least a portion of the cultivation step of permissive host cells is performed under constant perfusion, optionally wherein the perfusion at a slow steady rate. In some embodiments, the perfusion is or include an outflow of liquid e.g., conditioned (used) media, and an inflow of freshly prepared media. In certain embodiments, the perfusion replaces conditioned media with fresh media. In particular embodiments, culture of permissive host cells is started under conditions with no perfusion, and perfusion is started after a set and/or predetermined amount of time, such as or as about or at least 1 hour, 6 hours, 12 hours, 24 hours, 36 hours, 48 hours, 60 hours, 72 hours, or more than 72 hours after the start or initiation of the culture. In particular embodiments, perfusion is started when the density or concentration of the cells reaches a set or predetermined density or concentration. In particular embodiments, the perfusion is performed at different speeds during the culture. In some embodiments, the rate of the perfusion optionally depends on the density and/or concentration of the cultivated cells. In certain embodiments, the rate of perfusion is increased when the cells reach a set or predetermined density and/or concentration.

In some embodiments, a composition of host cells, such as host cells transfected with at least one plasmid for the production of a lentiviral vector particle, is cultivated in the presence of a surfactant. In particular embodiments, cultivating the cells of the composition reduces the amount of shear stress that may occur during the cultivation, e.g., due to mixing, rocking, motion, and/or perfusion.

In particular embodiments, the cultivation ends, such as by harvesting cells, when cells achieve a threshold amount, concentration, and/or expansion or after a fixed period of time. In some embodiments, the cultivation ends, such as by harvesting cells, when the cells achieve a threshold total amount of cells, e.g., threshold cell count. In some embodiments, the cultivation ends when the cells achieve a threshold viable amount of cells, e.g., threshold viable cell count.

In some of any embodiments, the threshold amount is 2.0×10⁶, 2.5×10⁶, 3.0×10⁶, 3.5×10⁶, or 4.0×10⁶. In some of any embodiments, the threshold amount is between 2.0×10⁶ and 2.5×10⁶, 2.0×10⁶ and 3.0×10⁶, 2.0×10⁶ and 3.5×10⁶, 2.0×10⁶ and 4.0×10⁶, 2.5×10⁶ and 3.0×10⁶, 2.5×10⁶ and 3.5×10⁶, 2.5×10⁶ and 4.0×10⁶, 3.0×10⁶ and 3.5×10⁶, 3.0×10⁶ and 4.0×10⁶, or 4.0×10⁶ and 4.5×10⁶, each inclusive.

In some embodiments, the threshold cell count is or is about or is at least of 3.0×10⁶ cells, or any of the foregoing threshold of viable cells. In particular embodiments, the cultivation ends when the cells achieve a threshold cell count. In some embodiments, the cultivation ends at, at about, or within 24 hours or more days, after the threshold cell count is achieved.

In some embodiments, the cultivation step is performed for the amount of time required for the cells to achieve a threshold amount, density, and/or expansion. In some embodiments, the cultivation is performed for or for about, or for less than, 24 hours.

In some embodiments, the cultivation is performed for at least a minimum amount of time, such as 1 day, 2 days, 3, days, 4, days, 5, days, 6, days, 7 days, 8 days, 9 days, or 10 days. In some embodiments, the cultivation is performed for at least a minimum amount of 11 days or more.

In some embodiments, the cells are harvested at or about 12 hours, at or about 24 hours, at or about 48 hours, at or about 72 hours or at or about 96 hours, at or about 4 days, at or about 5 days, at or about 6 days, or at or about 7 days after of initiation the introduction of the one more nucleic acids into the host cells. In some embodiments, the cells are harvested between at or about 12 hours and 96 hours, such as between at or about 12 hours and 72 hours, 12 hours and 48 hours, 12 hours and 24 hours, 24 hours and 96 hours, 24 hours and 72 hours, 24 hours and 48 hours, 48 hours and 96 hours, 48 hours and 72 hours or 72 hours and 96 hours, each inclusive, after initiation of the introduction of the one or more nucleic acids into the host cells. In some embodiments, the cells are harvested at or about 48 hours±12 hours after of initiation the introduction of the one more nucleic acids into the host cells. In some embodiments, the cells are harvested at or about 48 hours±6 hours after of initiation the introduction of the one more nucleic acids into the host cells. In some embodiments, the cells are harvested at or about 48 hours after of initiation the introduction of the one more nucleic acids into the host cells.

In some embodiments, the cells are monitored during the cultivation step. Monitoring may be performed, for example, to ascertain (e.g., measure, quantify) cell morphology, cell viability, cell death, and/or cell concentration (e.g., viable cell concentration). Monitoring may also be performed to ascertain media conditions, such as temperature, pH, saturated oxygen, and glucose. In some embodiments, the monitoring is performed manually, such as by a human operator. In some embodiments, the monitoring is performed by an automated system. In some embodiments, the monitoring is performed by a module, such as is consistent with the bioreactor used. The automated system may require minimal or no manual input to monitor the cultivated cells. In some embodiments, the monitoring is performed both manually and by an automated system.

In certain embodiments, the cells and/or media conditions are monitored by an automated system, such as system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the cells and/or media conditions are monitored by a module, such as an automated module, such that cells undergoing cultivation are not removed from the bioreactor until the end of the culture period.

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every hour for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 12 hours for the duration of the cultivating step. In some embodiments, the monitoring is performed at least once a day for the duration of the cultivating step. In some embodiments, the monitoring is performed at least once during the cultivating step.

In some embodiments, cell viability is characterized or determined. In some embodiments, cell concentration, density and/or number is characterized or determined. In some embodiments, viable cell concentration, viable cell number and/or viable cell density is characterized or determined.

In some embodiments, the cultivated cells are harvested, such as by automatic or manual methods, for example, by a human operator. In some embodiments, the cell are harvested when they reach a threshold of expansion. The threshold of expansion may depend on the total concentration, density and/or number of cultured cells determined by the automated system. Alternatively, the threshold of expansion may depend on the viable cell concentration, density and/or number.

In some embodiments, the harvested cells are formulated as described, such as in the presence of a pharmaceutically acceptable carrier, such as is described below in Section III. In some embodiments, the harvested cells are formulated in the presence of a cryoprotectant.

### III. Lipid Membrane Bound Particle Compositions and Methods of Use

The present disclosure also provides, in some aspects, a pharmaceutical composition comprising the lipid membrane bound particle composition described herein and pharmaceutically acceptable carrier. The pharmaceutical compositions can include any of the described non-cell particles. In particular embodiments, the lipid membrane bound particles are produced by methods provided herein.

In some embodiments, provided herein are the use of pharmaceutical compositions of the lipid membrane bound particle or salts thereof to practice the disclosed methods. Such a pharmaceutical composition may consist of at least one compound or conjugate of the invention or a salt thereof in a form suitable for administration to a subject, or the pharmaceutical composition may comprise at least one compound or conjugate of the invention or a salt thereof, and one or more pharmaceutically acceptable carriers, one or more additional ingredients, or some combination of these. In some embodiments, the compound or conjugate of the invention may be present in the pharmaceutical composition in the form of a physiologically acceptable salt, such as in combination with a physiologically acceptable cation or anion, as is well known in the art.

In some embodiments, the relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. In some embodiments, the composition may comprise between 0.1% and 100% (w/w) active ingredient.

In some embodiments, pharmaceutical compositions that are useful in the methods of the invention may be suitably developed for oral, rectal, vaginal, parenteral, topical, pulmonary, intranasal, buccal, ophthalmic, or another route of administration. In some embodiments, a composition useful within the methods of the invention may be directly administered to the skin, vagina or any other tissue of a mammal. In some embodiments, formulations include liposomal preparations, resealed erythrocytes containing the active ingredient, and immunologically based formulations. In some embodiments, the route(s) of administration will be readily apparent to the skilled artisan and will depend upon any number of factors including the type and severity of the disease being treated, the type and age of the veterinary or human subject being treated, and the like.

In some embodiments, the lipid membrane bound particles (e.g. paramyxovirus lipid particles) provided herein can be administered to a subject, e.g. a mammal, e.g. a human. In such embodiments, the subject may be at risk of, may have a symptom of, or may be diagnosed with or identified as having, a particular disease or condition.

In one embodiment, the subject has cancer. In one embodiment, the subject has an infectious disease. In some embodiments, the lipid membrane bound particle, such as a Nipah protein lipid particle, contains nucleic acid sequences encoding an exogenous agent for treating the disease or condition in the subject. For example, the exogenous agent is one that targets or is specific for a protein of a neoplastic cells and the lipid particle is administered to a subject for treating a tumor or cancer in the subject. In another example, the exogenous agent is an inflammatory mediator or immune molecule, such as a cytokine, and lipid membrane bound particle is administered to a subject for treating any condition in which it is desired to modulate (e.g. increase) the immune response, such as a cancer or infectious disease.

In some embodiments, the lipid membrane bound particle or composition comprising the same is administered in an effective amount or dose to effect treatment of the disease, condition or disorder. Provided herein are uses of any of the provided lipid membrane bound particles or composition comprising the same in such methods and treatments, and in the preparation of a medicament in order to carry out such therapeutic methods. In some embodiments, the methods are carried out by administering the lipid membrane bound particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent (i.e., exogenous agents such as is provided in Section I.B).

In some embodiments, the provided methods or uses involve administration of a pharmaceutical composition comprising oral, inhaled, transdermal or parenteral (including intravenous, intratumoral, intraperitoneal, intramuscular, intranodal, intracavity, and subcutaneous) administration. In some embodiments, the lipid membrane bound particle may be administered alone or formulated as a pharmaceutical composition. In some embodiments, the lipid membrane bound particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the lipid membrane bound particles may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, the lipid membrane bound particle composition comprising an exogenous agent, may be used to deliver such exogenous agent to a cell tissue or subject. In some embodiments, delivery of such exogenous agent by administration of a lipid membrane bound particle composition described herein may modify cellular protein expression levels. In certain embodiments, the administered composition directs upregulation of (via expression in the cell, delivery in the cell, or induction within the cell) of one or more exogenous agent (e.g., a polypeptide or gene) that provide a functional activity which is substantially absent or reduced in the cell in which the polypeptide is delivered. In some embodiments, the missing functional activity may be enzymatic, structural, or regulatory in nature.

In some of any embodiments, the lipid membrane bound particle composition mediates an effect on a target cell, and the effect lasts for at least 1, 2, 3, 4, 5, 6, or 7 days, 2, 3, or 4 weeks, or 1, 2, 3, 6, or 12 months. In some embodiments, the effect lasts for less than 1, 2, 3, 4, 5, 6, or 7 days, 2, 3, or 4 weeks, or 1, 2, 3, 6, or 12 months.

In some of any embodiments, the lipid membrane bound particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the lipid membrane bound particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the lipid membrane bound particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid membrane bound particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the lipid membrane bound particle particles is co-administered with an inhibitor of sypressyn, e.g., a siRNA or inhibitory antibody.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Examples of lipid particles include solid particles such as nanoparticles, viral-derived particles or cell-derived particles. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity and include 10%-150% or more of the activity of a full-length or wild-type F protein or G protein. Examples of biologically active portions of F and G proteins include truncations of the cytoplasmic domain, e.g. truncations of up to 1, 2, 3, 4, 5, 6, 7, 8, 9, 10, 11, 12, 13, 14, 15, 20, 25, 30, 35 or more contiguous amino acids, see e.g. Khetawat and Broder 2010 Virology Journal 7:312; Witting et al. 2013 Gene Therapy 20:997-1005; published international; patent application No. WO/2013/148327.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell.

As used herein, the term “specifically binds” to a target molecule, such as an antigen, means that a binding molecule, such as a an antibody or antigen-binding fragment thereof), reacts or associates more frequently, more rapidly, with greater duration and/or with greater affinity with a particular target molecule than it does with alternative molecules. A binding molecule, such as an antibody or antigen-binding fragment thereof, “specifically binds” to a target molecule if it binds with greater affinity, avidity, more readily, and/or with greater duration than it binds to other molecules. It is understood that a binding molecule, such as an antibody or antigen-binding fragment thereof), that specifically binds to a first target may or may not specifically bind to a second target. As such, “specific binding” does not necessarily require (although it can include) exclusive binding.

As used herein, the term “neutralizing,” or “neutralization” or grammatical variations thereof in relation to the binding agents refer to those that contain a binding domain (e.g. antibody or antigen-binding fragment thereof) that blocks or reduces at least one activity of a virus having exposed on its surface a viral surface protein to which the binding domain (e.g. antibody or antigen-binding fragment thereof) specifically binds. For example, neutralization can be achieved by inhibiting the attachment or adhesion of the virus to a target cell surface, e.g., by a binding domain (e.g. an antibody or antigen-binding fragment thereof) that binds directly to, or close by, the site responsible for the attachment or adhesion of the virus. Neutralizing activity includes activity to inhibit a virus from replication. A neutralizing activity may be measured in vitro and/or in vivo.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Amino acid substitutions may be introduced into an antibody of interest and the products screened for a desired activity, for example, retained/improved binding. Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a targeted lipid particle, refers to an agent that is neither comprised by nor encoded in the corresponding wild-type virus. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used here, “maintains pH” or wherein the “pH is maintained” refers to the pH being held within a specified range, e.g., ±0.05, ±0.10, ±0.20, or ±0.50 pH units over time after the pH has been equilibrated. To maintain a fixed pH, a solution such as sodium carbonate and/or CO₂ can be used to maintain the pH of the solution within the prescribed range.

As used here, “maintains DO” or wherein the “DO is maintained” refers to the level of dissolved oxygen (DO) being held within a narrow range, e.g., ±0.10%, ±1.0%, or ±10% over time after the DO has been equilibrated. To maintain a fixed DO, a gas such as air, nitrogen, and/or O₂ is used to maintain the desired level of dissolved oxygen.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

As used herein, a “promoter” refers to a cis- regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a “vehicle” refers to a biological carrier for delivering genes or proteins to cells to facilitate their recognition or uptake by cells. Examples of delivery vehicles include, but are not limited to, lipid and non-lipid particles, such as virus or virus like particles, liposomes, microparticles, nanoparticles, nanogels, dendrimer or dendrisomes.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of any of the provided embodiments with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### V. Exemplary Embodiments

Among the provided embodiments are:

1. A method of producing a lipid membrane bound particle, said method comprising culturing host cells comprising one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the pH of the medium during the culturing is maintained at a culture pH of 6.7 to 7.7,
wherein the lipid membrane bound particle produced by the method comprises a lipid bilayer and the one or more Paramyxovirus envelope protein or biologically active portion thereof are embedded in the lipid bilayer.

2. A method of producing a lipid membrane bound particle, said method comprising culturing host cells transfected with one or more nucleic acids for the production of a lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the pH of the medium during the culturing is maintained at a culture pH that is slightly basic,
wherein the lipid membrane bound particle produced by the method comprises a lipid bilayer enclosing a lumen and the one or more Paramyxovirus envelope protein or biologically active portion thereof are embedded in the lipid bilayer.

3. The method of embodiment 2, wherein at least one of the one or more nucleic acids for the production of the lipid membrane bound particle derived from a virus are stably expressed in the host cell, optionally wherein the one or more nucleic acids are integrated into the chromosome of the host cell.

4. The method of embodiment 1 or embodiment 2, wherein the nucleic acid encoding the one or more Paramyxovirus envelope protein or a biologically active portion thereof is introduced into the cell, optionally by transfection of the one or more nucleic acids.

5. A method of producing a lipid membrane bound particle, said method comprising culturing host cells transfected with one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the culturing the pH of the medium during the culturing is maintained at a culture pH of 6.7 to 7.7,
wherein the lipid membrane bound particle comprises a lipid bilayer and the one or more Paramyxovirus envelope protein or biologically active portion thereof are embedded in the lipid bilayer.

6. A method of producing a lipid membrane bound particle, said method comprising culturing host cells transfected with one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the culturing the pH of the medium during the culturing is maintained at a culture that is slightly basic,
wherein the lipid membrane bound particle comprises a lipid bilayer and the one or more Paramyxovirus envelope protein or biologically active portion thereof are embedded in the lipid bilayer.

7. A method of culturing a cell, said method comprising culturing host cells comprising one or more nucleic acids for the production of the lipid membrane bound particle derived from a virus, wherein one of the one or more nucleic acids further encodes one or more Paramyxovirus envelope protein or a biologically active portion thereof, and wherein the pH of the medium during the culturing is maintained at a culture pH of 6.7 to 7.7.

8. A method of culturing a cell, said method comprising culturing host cost cells comprising one or more nucleic acids for the production of the lipid membrane bound particle derived from a virus, wherein one of the one or more nucleic acids further encodes one or more Paramyxovirus envelope protein or a biologically active portion thereof, and wherein the pH of the medium during the culturing is maintained at a culture pH that is slightly basic.

9. The method of embodiment 7 or embodiment 8, wherein the one or more nucleic acids for the production of the lipid membrane bound particle derived from a virus are stably expressed in the host cell, optionally wherein the one or more nucleic acids are integrated into the chromosome of the host cell.

10. The method of any of embodiments 7-9, wherein the nucleic acid encoding the one or more Paramyxovirus envelope protein or a biologically active portion thereof is introduced into the cell, optionally by transfection of the one or more nucleic acids.

11. The method of any of embodiments 1-4, 6, 8-10, wherein the pH of the medium during the culturing is maintained at a culture pH of 6.8 to 7.5

12. The method of any of embodiments 1-4, 6, 8-10, wherein the pH of the medium during the culturing is maintained at a culture pH of 6.9 to 7.4

13. The method of any of embodiments 1-12, wherein the pH of the medium during the culturing is maintained at a culture pH of 7 to 7.3.

14. The method of any one of embodiment 1-13, wherein the lipid membrane bound particle is a viral-like particle (VLP), or vector particle derived from a retrovirus.

15. The method of any one of embodiments 1-14, wherein the lipid membrane bound particle is a VLP or vector particle derived from a lentivirus.

16. A method of producing a lentiviral vector particle, said method comprising culturing host cells transfected with one or more nucleic acids for the production of a lentiviral lipid membrane bound particle, wherein one of the one or more nucleic acids further encodes one or more Paramyxovirus envelope protein or a biologically active portion thereof, and wherein the culturing is carried out in slightly basic medium under conditions for producing the lentiviral lipid membrane bound particle by the host cells.

17. The method of any one of embodiments 1-16, wherein the host cell is a mammalian cell, optionally wherein the host cell is selected from the group comprising HEK293 or 293T cells.

18. The method of embodiments 16 or 17, wherein the culturing is carried out in medium with a culture pH of 7.05-7.7.

19. The method of any one of embodiments 16-18, wherein the culturing is carried out in medium with a culture pH of 7.1-7.3.

20. The method of any of embodiments 1-19, wherein the culture pH is allowed to change at a pH set point with a deadband from 0.05, 0.10, 0.15, 0.20, 0.25, 0.30, 0.35, 0.40, 0.45 or 0.50 or any value between any of the foregoing.

21. The method of any of embodiments 1-20, wherein the culture pH is selected from: (i) pH 7.05±0.05; (ii) pH 7.15±0.05; (iii) pH 7.20±0.05; (iv) pH 7.35±0.05; (v) pH 7.05±0.10; (vi) pH 7.15±0.10; (vii) pH 7.20±0.10; (viii) pH 7.35±0.10; (ix) pH 7.05±0.15; (x) pH 7.15±0.15; (xi) pH 7.20±0.15; or (xii) pH 7.35±0.15.

22. The method of any of embodiments 1-21, wherein the culture pH is at or about 7.05, at or about 7.1, at or about 7.15, at or about 7.2, at or about 7.3, at or about 7.35.

23. The method of any of embodiments 1-22, wherein the culture pH is at or about 7.1

24. The method of any of embodiments 1-22, wherein the culture pH is at or about 7.2.

25. The method of any of embodiments 1-22, wherein the culture pH is at or about 7.3.

26. The method of any of embodiments 1-25, wherein the medium has a dissolved oxygen concentration between 30 and 60 percent saturation.

27. The method of any of embodiment 1-26, wherein the medium has a dissolved oxygen concentration of 20, 30, 40, 50, or 60 percent saturation, or any value between any of the foregoing.

28. The method of any of embodiments 1-27, wherein the culturing is carried out in a bioreactor.

29. The method of embodiment 28, wherein the bioreactor is a stirred-tank bioreactor.

30. The method of any of embodiment 1-29, wherein the culturing is carried out in a volume of at least 1 L.

31. The method of any of embodiment 1-30, wherein the culturing is carried out in a volume of at least 5 L.

32. The method of any of embodiments 1-30, wherein the culturing is carried out in a volume between at or about 1 L-5 L, between at or about 5 L-10 L, between at or about 10 L-20 L, between at or about 20 L-50 L, between at or about 50-100 L, or between at or about 100-200 L.

33. The method of any of embodiments 1-28, wherein the method further comprises monitoring the pH of the medium and, optionally adjusting the pH to maintain the culture pH of the medium.

34. The method of any of embodiments 28-33, wherein the bioreactor comprises a pH adjustment module, wherein the pH adjustment module monitors the pH of the medium during the culturing.

35. The method of any of embodiments 28-34, wherein the bioreactor comprises a pH adjustment module, wherein the pH adjustment module maintains the pH of the medium during the culturing.

36. The method of embodiments 1-35, wherein the one or more Paramyxovirus envelope proteins have fusogenic activity.

37. The method of embodiments 1-36, wherein the native binding tropism of the one or more of the Paramyxovirus envelope proteins is reduced.

38. The method of embodiment 1-37, wherein the one or more Paramyxovirus envelope proteins is derived from an H protein molecule or a biologically active portion thereof from a Paramyxovirus and/or an HN protein molecule or a biologically active portion thereof from a Paramyxovirus.

39. The method of embodiment 1-38, wherein the one or more Paramyxovirus envelope proteins comprises an F protein molecule or a biologically active portion thereof and/or a glycoprotein G (G protein) or a biologically active portion thereof.

40. The method of embodiment 1-37, wherein the one or more Paramyxovirus envelope proteins comprises an F protein molecule or a biologically active portion thereof from a Paramyxovirus and a glycoprotein G (G protein) or a biologically active portion thereof from a Paramyxovirus.

41. The method of any of embodiments 1-40, wherein the paramyxovirus is a henipavirus.

42. The method of any of embodiments 1-41, wherein the paramyxovirus is Measles morbillivirus.

43. The method of any of embodiments 1-41, wherein the paramyxovirus is a Hendra virus.

44. The method of any of embodiments 1-41, wherein the paramyxovirus is Nipah virus.

45. The method of any of embodiments 39-41 and 44, wherein the F protein or the biologically active portion thereof is a wild-type Nipah virus F (NiV-F) protein or is a functionally active variant or biologically active portion thereof.

46. The method of any of embodiments 39-41, 44 or 45 wherein the F protein molecule or a biologically active portion thereof is a NiV-F protein that has the amino acid sequence set forth in SEQ ID NO: 4 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

47. The method of any of embodiments 39-41 and 44-46, wherein the NiV-F protein is a biologically active portion that is truncated and lacks up to 40 contiguous amino acid residues at or near the C-terminus of the wild-type NiV-F protein (SEQ ID NO:4).

48. The method of any of embodiments 39-41 and 44-47, wherein the NiV-F protein is a biologically active portion that is truncated at the C-terminus of wild-type NiV-F and has the sequence set forth in any of SEQ ID NOS: 5-15 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 5-15.

49. The method of any of embodiments 39-41 and 44-48, wherein the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:4.

50. The method of any of embodiments 39-41 and 44-49, wherein the F protein is a NiV-F protein that is a biologically active portion that has a 20 amino acid truncation at or near the C-terminus of the wild-type NiV-F protein (SEQ ID NO:4).

51. The method of embodiment 50, wherein the NiV-F protein has an amino acid sequence set forth in SEQ ID NO:37 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 37.

52. The method of any of embodiments 39-41 and 44-49, wherein the F protein is a NiV-F protein that is a biologically active portion thereof that has a 22 amino acid truncation at or near the C-terminus of the wild-type NiV-F protein (SEQ ID NO:4).

53. The method of embodiment 52, wherein the NiV-F protein has an amino acid sequence set forth in SEQ ID NO:32 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

54. The method of embodiment 52 or embodiment 53, wherein the NiV-F protein has an amino acid sequence set forth in SEQ ID NO:33 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 33.

55. The method of any of embodiments 38-41 and 44-54, wherein the F protein or the biologically active portion thereof is a NiV-F protein that is a functionally active variant that comprises a point mutation on an N-linked glycosylation site of the wild-type NiV-F protein (SEQ ID NO:4) or a biologically active potion thereof.

56. The method of any of embodiments 38-41 and 44-54, wherein the F protein or the biologically active portion thereof is a NiV-F protein that is a functionally active variant that comprises:
i) a 20 amino acid truncation at or near the C-terminus of the wild-type NiV-F protein (SEQ ID NO:4); and/or
ii) a point mutation on an N-linked glycosylation site.

57. The method of embodiment 56, wherein the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

58. The method of any of embodiments 39-41 and 43-57, wherein the G protein or the biologically active portion thereof is a wild-type Nipah virus G (NiV-G) protein or a Hendra virus G protein or is a functionally active variant or biologically active portion thereof.

59. The method of any of embodiments 39-41 and 44-58, wherein the G protein or the biologically active portion thereof is a wild-type NiV-G protein or a functionally active variant or biologically active portion thereof.

60. The method of any of embodiments 39-41 and 44-59, wherein the G protein or the biologically active portion thereof is a mutant NiV-G protein that is modified to exhibit reduced native binding tropism.

61. The method of any of embodiments 39-41 and 44-60, wherein the G protein or the biologically active portion thereof is a mutant NiV-G protein that exhibits reduced binding to Ephrin B2 or Ephrin B3.

62. The method of any of embodiments 39-41 and 44-61, wherein the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:1.

63. The method of any of embodiments 39-41 and 44-62, wherein the NiV-G protein is a biologically active portion that is truncated and lacks up to 40 contiguous amino acid residues at or near the N-terminus of the wild-type NiV-G protein (SEQ ID NO:1).

64. The method of any of embodiments 39-41 and 44-63, wherein the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:1, SEQ ID NO:2, or SEQ ID NO: 5.

65. The method of any of embodiments 39-41 and 44-64, wherein the NiV-G protein is a biologically active portion that is truncated at the N-terminus of wild-type NiV-G and has the sequence set forth in any of SEQ ID NOS: 2, 5, or 6 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs 1, 2, or 5.

66. The method of any of embodiments 39-41 and 44-65, wherein the G protein molecule or a biologically active portion thereof NiV-G protein has the amino acid sequence set forth in SEQ ID NO: 1 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1.

67. The method of any of embodiments 39-41 and 44-65, wherein the G protein or the biologically active portion thereof is a mutant NiV-G protein having the amino acid sequence set forth in SEQ ID NO: 2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:2.

68. The method of any of embodiments 39-41 and 44-65, wherein the G protein or the biologically active portion thereof is a mutant NiV-G protein having the amino acid sequence set forth in SEQ ID NO: 5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5.

69. The method of any of embodiments 39-41, 44-67, wherein the F protein comprises the sequence set forth in SEQ ID NO. 32 and the G protein comprises the sequence set forth in SEQ ID NO. 34.

70. The method of any of embodiments 1-69, wherein at least one of the one or more Paramyxovirus envelope proteins are linked to a secondary moiety that is a targeting domain or a functional domain.

71. The method of embodiment 70, wherein the at least one of the one or more Paramyxovirus is a glycoprotein G (G protein) or a biologically active portion thereof and the G protein or biologically active portion thereof is linked to the secondary moiety.

72. The method of embodiment 70 or embodiment 71, wherein the secondary moiety is a functional domain and the functional domain is selected from a cytokine, growth factor, hormone, neurotransmitter, receptor, or apoptosis ligand.

73. The method of embodiment 70 or embodiment 71, wherein the secondary moiety is a targeting domain and the targeting domain is specific for a cell surface receptor on a target cell.

74. The method of any of embodiment 70, wherein the targeting domain is a Design ankyrin repeat proteins (DARPin), a single domain antibody (sdAb), a single chain variable fragment (scFv), or an antigen-binding fibronectin type III (Fn3) scaffold.

75. The method of any one of embodiments 70-74, wherein the at least one of the one or more Paramyxovirus envelope proteins and the secondary moiety are directly linked.

76. The method of any one of embodiments 70-74, wherein the at least one of the one or more Paramyxovirus envelope proteins and secondary moiety are indirectly linked via a linker.

77. The method of embodiment 76, wherein the linker is a peptide linker.

78. The method of embodiment 77, wherein the peptide linker is (GmS)n (SEQ ID NO: 11), wherein each of m and n is an integer between 1 to 4, inclusive.

79. The method of any of embodiments 1-78, wherein one of the one or more nucleic acids for the production of the lipid membrane bound particle derived from a virus encodes an expression cassette comprising at least one retroviral gene, optionally wherein the at least one retroviral gene is a lentiviral gene.

80. The method of embodiment 79, wherein the at least one retroviral gene is a lentiviral gene selected from the group comprising gag, rev, and/or pol.

81. The method of embodiment 79 or embodiment 80, wherein the at least one retroviral gene is stably expressed in the host cell, optionally wherein the one or more nucleic acids are integrated into the chromosome of the host cell.

82. The method of any of embodiments 1-81, wherein one of the one or more nucleic acids for the production of the lipid membrane bound particle derived from a virus further encodes a transgene.

83. The method of any of embodiments 1-82, wherein one of the one or more nucleic acids for the production of the lipid membrane bound particle derived from a virus does not encode VSV-G.

84. The method of embodiment 15-83, wherein the lipid membrane bound particle derived from a lentivirus or the lentiviral lipid membrane bound particle is a VLP or vector particle derived from HIV-1.

85. The method of any of embodiments 15-83, wherein the lipid membrane bound particle derived from a lentivirus or the lentiviral lipid membrane bound particle is a VLP or vector particle derived from gamma-retrovirus (GaL-V).

86. The method of any of embodiments 1-6, 11-15, 23-85, wherein the method further comprises purifying the lipid membrane bound particle comprising the one or more Paramyxovirus envelope protein or biologically active portion thereof.

87. The method of any of embodiments 7-10, 16-19, wherein the method further comprises collecting the supernatant from the cell culture, said supernatant containing the lipid membrane bound particle produced by the host cells.

88. The method of embodiment 87, further comprising clarification and/or concentration of lipid membrane bound particles.

89. The method of any of embodiments 1-6, 11-15, 23-86, wherein the method further comprises purifying the lentiviral vector particles comprising the one or more Paramyxovirus envelope protein or biologically active portion thereof.

90. The method of embodiment 7-10, 16-19, 87-88, wherein the method further comprises collecting the supernatant from the cell culture, said supernatant containing the lentiviral vector particles produced by the host cells.

91. The method of embodiment 90, further comprising clarification and/or concentration of lentiviral vector particle.

92. The method of any of embodiments 88 or 91, wherein the clarification and/or concentration is by centrifugation.

93. The method of any of embodiments 88 or 91, wherein the clarification and/or concentration is by dialysis and/or filtration.

94. A composition comprising lipid membrane bound particles produced by the method of any of embodiments 1-93.

95. A composition comprising lentiviral vector particles produced by the method of any of embodiments 16-93.

96. The composition of any of embodiments 94-95, wherein the ratio of infectious titer to physical titer of the lipid membrane bound particles or lentiviral vector particles is 10,000:1, 5,000:1, 2,500:1, 1,000:1, 100:1 or lower.

97. The composition of any of embodiments 94-96, wherein the ratio of infectious titer to physical titer of the lipid membrane bound particles or lentiviral vector particles is between 10,000:1 and 100:1, 10,000:1 and 1,1000:1, 10:000:′ and 2,500:1, 1:10,000 and 5000:1, 5,000:1 and 100:1, 5,000:1 and 1000:1, 5000:1 and 2500:1, 2500:1 and 100:1, 2500:1 and 1000:1, or 1000:1 and 100:1.

98. The composition of any of embodiments 94-97, wherein the ratio of infectious titer to physical titer of the lipid membrane bound particles or lentiviral vector particles is between 10,000:1 and 1000:1.

99. The composition of any of embodiments 94-98, wherein the ratio of infectious titer to physical titer of the lipid membrane bound particles or lentiviral vector particles is between 10,000:1 and 5000:1.

100. A cell culture system, wherein said system comprises a vessel comprising cell culture medium and host cells, said vessel further comprising a pH monitoring module contacting cell culture medium comprising host cells transfected with one or more nucleic acids for the production of a lipid membrane bound particle derived from a virus, wherein at least one the one of the one or more nucleic acids further encodes an envelope protein derived from a Paramyxovirus.

101. The cell culture system of embodiment 100, wherein the lipid membrane bound particle is a retroviral vector particle, optionally a lentiviral vector particle.

102. A cell culture system, wherein said system comprises a vessel comprising cell culture medium and host cells, said vessel further comprising a pH monitoring module contacting the cell culture medium comprising host cells transfected with one or more nucleic acids for the production of a lentiviral vector particle, wherein the one or more nucleic acids further encodes an envelope protein derived from a Paramyxovirus.

103. A cell culture system, wherein said system comprises a vessel comprising at least 5 L of cell culture medium comprising host cells transfected with one or more nucleic acids for the production of a lipid membrane bound particle derived from a virus, wherein the at least one of the one or more nucleic acids further encodes an envelope protein derived from a Paramyxovirus.

104. The cell culture system of any of embodiments 100-103, wherein said vessel comprises a bioreactor.

105. The cell culture system of any of embodiments 100-104, wherein the cell culture system is for culturing the host cells under conditions for producing the lipid membrane bound particle by the host cells.

106. The cell culture system of any of embodiments 100-105, wherein the system further comprises a pH adjustment module, wherein the pH adjustment module monitors the pH of the medium during the culturing.

107. The cell culture system of any of embodiments 100-105, wherein the system further comprises a pH adjustment module, wherein the pH adjustment module maintains the pH of the medium during the culturing.

108. The cell culture system of embodiments 106 or 107, wherein the medium is maintained at a pH of 6.7 to 7.7.

109. The cell culture system of embodiments 106 or 107, wherein the medium is maintained at a pH of 7.1-7.3.

110. The cell culture system of any of embodiments 106-109, wherein the medium is maintained at a pH with a deadband from 0.05, 0.10, 0.15, 0.20, 0.25, 0.30, 0.35, 0.40, 0.45 or 0.50 or any value between any of the foregoing.

111. The cell culture system of any of embodiments 106-110, wherein the medium is maintained at a pH selected from: (i) pH 7.05±0.05; (ii) pH 7.15±0.05; (iii) pH 7.20±0.05; (iv) pH 7.35±0.05; (v) pH 7.05±0.10; (vi) pH 7.15±0.10; (vii) pH 7.20±0.10; (viii) pH 7.35±0.10; (ix) pH 7.05±0.15; (x) pH 7.15±0.15; (xi) pH 7.20±0.15; or (xii) pH 7.35±0.15.

112. The cell culture system of any of embodiments 106-111, wherein the culture pH is at or about 7.05, at or about 7.1, at or about 7.15, at or about 7.2, at or about 7.3, at or about 7.35.

113. The cell culture system of embodiments 106-112, wherein the medium has a pH of at or about 7.1

114. The cell culture system of embodiments 106-112, wherein the medium has a pH of at or about 7.2.

115. The cell culture system of embodiments 106-112, wherein the medium has a pH of at or about 7.3.

116. The cell culture system of embodiments 100-115, wherein the medium has a dissolved oxygen concentration between 40 and 60 percent saturation.

117. The cell culture system of embodiments 100-116, wherein the system further comprises a glucose monitor.

118. The cell culture system of embodiments 100-117, wherein the system further comprises a module for controlling stirring speed.

119. The cell culture system of embodiments 100-118, wherein the one or more Paramyxovirus envelope proteins have fusogenic activity.

120. The cell culture system of embodiments 100-119, wherein the native binding tropism of the one or more of the Paramyxovirus envelope proteins is reduced.

121. The cell culture system of embodiments 100-120, wherein the one or more Paramyxovirus envelope proteins is derived from an H protein molecule or a biologically active portion thereof from a Paramyxovirus and/or an HN protein molecule or a biologically active portion thereof from a Paramyxovirus.

122. The cell culture system of embodiments 100-120, wherein the one or more Paramyxovirus envelope proteins comprises an F protein molecule or a biologically active portion thereof and/or a glycoprotein G (G protein) or a biologically active portion thereof.

123. The cell culture system of embodiments 100-120, wherein the one or more Paramyxovirus envelope proteins comprises an F protein molecule or a biologically active portion thereof from a Paramyxovirus and a glycoprotein G (G protein) or a biologically active portion thereof from a Paramyxovirus.

124. The cell culture system of embodiments 100-123, wherein the paramyxovirus is a henipavirus.

125. The cell culture system of embodiments 100-123, wherein the paramyxovirus is Measles morbillivirus.

126. The cell culture system of embodiments 100-124, wherein the paramyxovirus is a Hendra virus.

127. The cell culture system of embodiments 100-124, wherein the paramyxovirus is Nipah virus.

128. The cell culture system of embodiments 100-124, 127, wherein the F protein or the biologically active portion thereof is a wild-type Nipah virus F (NiV-F) protein or is a functionally active variant or biologically active portion thereof

129. The cell culture system of embodiments 100-124, 127, or 128, wherein the F protein molecule or a biologically active portion thereof is a NiV-F protein that has the amino acid sequence set forth in SEQ ID NO: 4 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

130. The cell culture system of embodiments 100-124, 127-129, wherein the NiV-F protein is a biologically active portion that is truncated and lacks up to 40 contiguous amino acid residues at or near the C-terminus of the wild-type NiV-F protein (SEQ ID NO:4).

131. The cell culture system of embodiments 100-124, 127-130, wherein the NiV-F protein is a biologically active portion that is truncated at the C-terminus of wild-type NiV-F and has the sequence set forth in any of SEQ ID NO. 32 or SEQ ID NO. 33 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO. 32 or SEQ ID NO. 33.

132. The cell culture system of embodiments 100-124, 127-131, wherein the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:4.

133. The cell culture system of embodiments 100-124, 127-132, wherein the F protein is a NiV-F protein that is a biologically active portion that has a 20 amino acid truncation at or near the C-terminus of the wild-type NiV-F protein (SEQ ID NO:4).

134. The cell culture system of embodiment 133, wherein the NiV-F protein has an amino acid sequence set forth in SEQ ID NO:37 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 37.

135. The cell culture system of embodiments 100-124, 127-132, wherein the F protein is a NiV-F protein that is a biologically active portion thereof that has a 22 amino acid truncation at or near the C-terminus of the wild-type NiV-F protein (SEQ ID NO:4).

136. The cell culture system of embodiment 135, wherein the NiV-F protein has an amino acid sequence set forth in SEQ ID NO:38 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 38.

137. The cell culture system of embodiment 135, wherein the NiV-F protein has an amino acid sequence set forth in SEQ ID NO:36 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36.

138. The cell culture system of any of embodiments 100-124, 127-137, wherein the F protein or the biologically active portion thereof is a NiV-F protein that is a functionally active variant that comprises a point mutation on an N-linked glycosylation site of the wild-type NiV-F protein (SEQ ID NO:4) or a biologically active potion thereof.

139. The cell culture system of any of embodiments 100-124, 127-137, wherein the F protein or the biologically active portion thereof is a NiV-F protein that is a functionally active variant that comprises:
i) a 20 amino acid truncation at or near the C-terminus of the wild-type NiV-F protein (SEQ ID NO:4); and/or
ii) a point mutation on an N-linked glycosylation site.

140. The cell culture system of embodiment 139, wherein the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

141. The cell culture system of any of embodiments 100-124, 127-140, wherein the G protein or the biologically active portion thereof is a wild-type Nipah virus G (NiV-G) protein or a Hendra virus G protein or is a functionally active variant or biologically active portion thereof.

142. The cell culture system of any of embodiments 100-124, 127-140, wherein the G protein or the biologically active portion thereof is a wild-type NiV-G protein or a functionally active variant or biologically active portion thereof.

143. The cell culture system of any of embodiments 100-124, 127-142, wherein the G protein or the biologically active portion thereof is a mutant NiV-G protein that is modified to exhibit reduced native binding tropism.

144. The cell culture system of any of embodiments 100-124, 127-143, wherein the G protein or the biologically active portion thereof is a mutant NiV-G protein that exhibits reduced binding to Ephrin B2 or Ephrin B3.

145. The cell culture system of any of embodiments 100-124, 127-144, wherein the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:1.

146. The cell culture system of any of embodiments 100-124, 127-145, wherein the NiV-G protein is a biologically active portion that is truncated and lacks up to 40 contiguous amino acid residues at or near the N-terminus of the wild-type NiV-G protein (SEQ ID NO:1).

147. The cell culture system of any of embodiments 100-124, 127-146, wherein the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:1, SEQ ID NO:2, or SEQ ID NO: 5.

148. The cell culture system of any of embodiments 100-124, 127-147, wherein the NiV-G protein is a biologically active portion that is truncated at the N-terminus of wild-type NiV-G and has the sequence set forth in any of SEQ ID NOS: 1, 2, or 5 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 1, 2, or 5.

149. The cell culture system of any of embodiments 100-124, 127-148, wherein the G protein molecule or a biologically active portion thereof NiV-G protein has the amino acid sequence set forth in SEQ ID NO: 1 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1.

150. The cell culture system of any of embodiments 100-124, 127-148, wherein the G protein or the biologically active portion thereof is a mutant NiV-G protein having the amino acid sequence set forth in SEQ ID NO: 2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:2.

151. The cell culture system of any of embodiments 100-124, 127-148, wherein the G protein or the biologically active portion thereof is a mutant NiV-G protein having the amino acid sequence set forth in SEQ ID NO: 5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5.

152. The cell culture system of any of embodiments 100-124, 122-124, 127-131, 135-148, 150 wherein the F protein comprises the sequence set forth in SEQ ID NO. 32 and the G protein comprises the sequence set forth in SEQ ID NO. 34.

153. The cell culture system of any of embodiments 100-124, wherein at least one of the one or more Paramyxovirus envelope proteins are linked to a secondary moiety that is a targeting domain or a functional domain.

154. The cell culture system of embodiment 153, wherein the at least one of the one or more Paramyxovirus is a glycoprotein G (G protein) or a biologically active portion thereof and the G protein or biologically active portion thereof is linked to the secondary moiety.

155. The cell culture system of embodiment 153 or embodiment 154, wherein the secondary moiety is a functional domain and the functional domain is selected from a cytokine, growth factor, hormone, neurotransmitter, receptor, or apoptosis ligand.

156. The cell culture system of embodiment 153 or embodiment 155, wherein the secondary moiety is a targeting domain and the targeting domain is specific for a cell surface receptor on a target cell.

157. The cell culture system of any of embodiment 153, wherein the targeting domain is a Design ankyrin repeat proteins (DARPin), a single domain antibody (sdAb), a single chain variable fragment (scFv), a VHH antibody (nanobody), or an antigen-binding fibronectin type III (Fn3) scaffold.

158. The cell culture system of any one of embodiments 153-157, wherein the at least one of the one or more Paramyxovirus envelope proteins and the secondary moiety are directly linked.

159. The cell culture system of any one of embodiments 153-157, wherein the at least one of the one or more Paramyxovirus envelope proteins and secondary moiety are indirectly linked via a linker.

160. The cell culture system of embodiment 159, wherein the linker is a peptide linker.

161. The cell culture system of embodiment 160, wherein the peptide linker is (GₘS)ₙ (SEQ ID NO: 11), wherein each of m and n is an integer between 1 to 4, inclusive.

162. A method of producing a lipid membrane bound particle, said method comprising culturing host cost cells in the cell culture system of any of embodiments 157, wherein said host cells comprise one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid bilayer.

163. A method of producing a lipid membrane bound particle, said method comprising culturing host cost cells in the cell culture system of any of embodiments 96-157, wherein said host cells are transfected with one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid bilayer.

164. A method of culturing a cell, said method comprising culturing host cells in the cell culture system of any of embodiments 100-161, wherein said host cells comprise one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid

165. A method of culturing a cell, said method comprising culturing host cells in the cell culture system of any of embodiments 100-161, wherein said host cells are transfected with one or more nucleic acids for the production of a lipid membrane bound particle derived from a virus, wherein one of the one or more nucleic acids further encodes one or more Paramyxovirus envelope protein or a biologically active portion thereof, and wherein the culturing is carried out in medium with a culture pH that is slightly basic under conditions for producing the lipid membrane bound particle by the host cells, wherein the lipid membrane bound particle comprises a lipid bilayer enclosing a lumen and the one or more Paramyxovirus envelope protein or biologically active portion thereof are embedded in the lipid bilayer.

### VI. EXAMPLES

The following examples are included for illustrative purposes only and are not intended to limit the scope of the invention.

### Example 1—Effect of pH and Dissolved Oxygen on Functional Titer of Pseudotyped Lentiviral Vectors

To determine the effect of pH and dissolved oxygen concentration (DO) on production of VSV-G and retargeted Nipah pseudotyped lentiviral vectors, a response surface experiment was designed for each pseudotype. For VSV-G pseudotype, a central composite design experiment was performed in which pH was varied from 6.82 to 7.38, and DO was varied from 4.5% to 55.5%. For Nipah pseudotype, a central composite design experiment was performed in which pH was varied from 6.75 to 7.45, and DO was varied from 4.5% to 55.5%. Exemplary conditions for these experiments are shown in Table 3 below. By varying pH and DO independently using the design of experiments (DOE) techniques of response surface methodology, the effects of each parameter can be assayed independently, as well as interactions between the parameters.

TABLE 3
Exemplary conditions for
Composite Design Experiments

| | | |
|---|---|---|
| Reactor | pH Set Point | DO Setpoint |

VSV-G Pseudotype

| | | |
|---|---|---|
| R101 | 7.10 | 30.0 |
| R102 | 7.30 | 48.0 |
| R103 | 6.82 | 30.0 |
| R104 | 7.10 | 55.5 |
| R105 | 7.38 | 30.0 |
| R106 | 6.90 | 48.0 |
| R107 | 7.10 | 4.5 |
| R108 | 7.10 | 30.0 |
| R201 | 7.30 | 12.0 |
| R202 | 6.90 | 12.0 |

Nipah Pseudotype

| | | |
|---|---|---|
| R101 | 6.75 | 30.0 |
| R102 | 6.85 | 12.0 |
| R103 | 7.45 | 30.0 |
| R104 | 7.10 | 30.0 |
| R105 | 7.10 | 4.5 |
| R106 | 7.10 | 55.5 |
| R107 | 7.10 | 30.0 |
| R108 | 7.35 | 12.0 |
| R201 | 7.35 | 48.0 |
| R202 | 6.85 | 48.0 |

For viral vector production, HEK293 producer cells were grown in 3 L bioreactors (Mobius 3 L Single-use Bioreactor, EMD Millipore) and transfected with plasmids expressing viral vector proteins (gag/pol, rev) and a GFP transgene transfer plasmid. Envelope proteins were provided as plasmids expressing VSV-G or Nipah F protein and a CD8-retargeted Nipah G protein (see US 2019/0144885, incorporated by reference herein). The CD8-retargered Nipah G (NiV-G) protein contained an anti-CD8 scFv as a fusion with the exemplary NiV-G sequence GcA34 (Bender et al. 2016 PLoS Pathol 12(6):e1005641; set forth in SEQ ID NO:34), and the Nipah F (NiV-F) protein was the exemplary NiV-F sequence NivFdel22 (SEQ ID NO:33; or SEQ ID NO:38 without a signal sequence, i.e. see also SEQ ID NO:32; Bender et al. 2016 PLoS). pH and DO were monitored by using probes inserted into the bioreactor. pH control was provided by sodium carbonate and CO2, and DO control was provided by 02, air, and nitrogen. Cell growth and viability were monitored during production. Following viral vector production, the cell culture was centrifuged to pellet the cells and the supernatant containing crude virus was collected. The crude viral supernatant was tested for functional titer on SupT1 cells, which are permissive to transduction by both VSV-G and CD8-retargeted pseudotyped vectors.

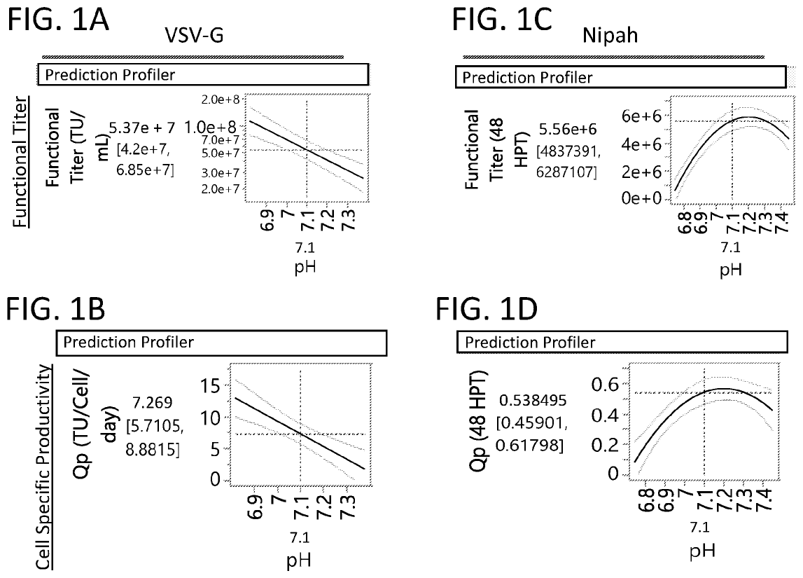

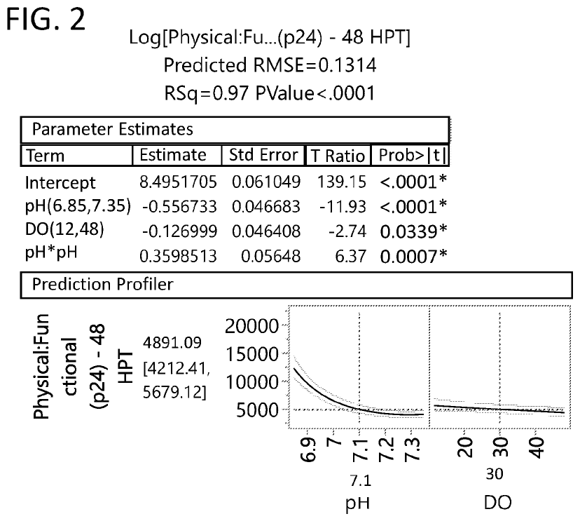

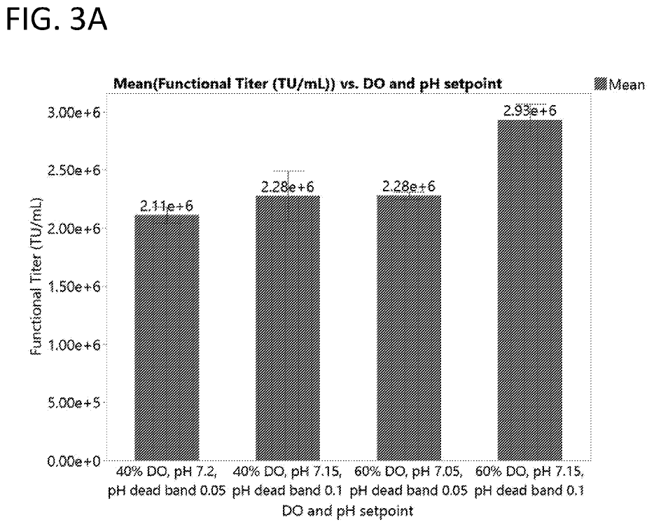

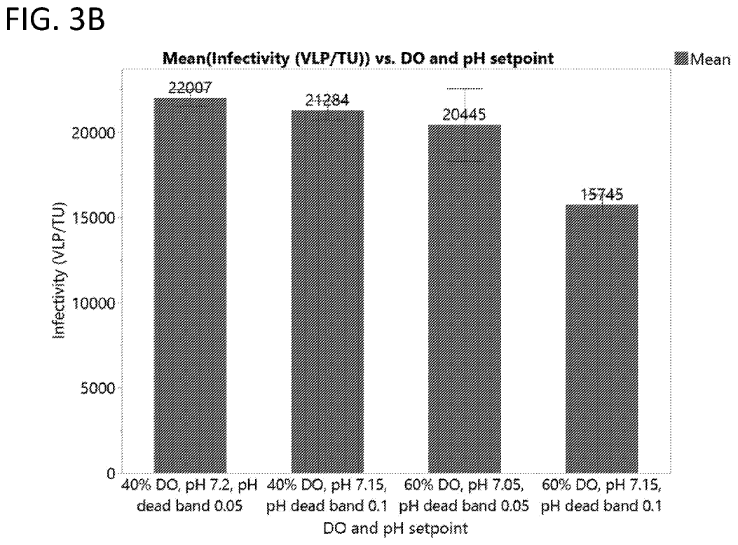

Regression analysis was performed to determine the effects of DO and pH on viral production. For both VSV-G and CD8-retargeted pseudotyped vectors, functional titer increased with increasing DO concentration, reaching a predicted maximum at ˜40%. Similarly, cell growth was observed to increase with increasing pH for both pseudotypes. However, viral production of the two pseudotypes responded very differently to pH. Functional titer and cell-specific productivity of VSV-G pseudotyped vectors improved with decreasing pH, with the greatest effect observed at pH 6.82 (the lowest pH tested) (FIGS. 1A-1B). This is consistent with previous reports (see, e.g., U.S. Pat. No. 10,125,352). For CD8-retargeted Nipah pseudotyped vector, functional titer and cell-specific productivity (Qp) were surprisingly found to increase with increasing pH, to a maximum at ˜pH 7.2 (FIGS. 1C-1D). Additionally, the ratio of non-infectious (measured by p24) to infectious particles (TU) decreased with increasing pH to 7.2 (FIG. 2). Similar results of increased production and decreased non-infectious:infectious particle ratio at about pH 7.0-7.2 were seen in follow-up experiments using other retargeted vectors with VHH or additional scFv binding domains. These additional observations included additional CD8 binding domain, ASGR1 binding domains, and vectors carrying transgenes other than GFP, such as chimeric antigen receptors. Other Henipavirus and Paramyxovirus family members are predicted to have similar pH responses.

This example demonstrates that improved production of lentiviral vectors pseudotyped with Paramyxovirus fusogens can be achieved by culturing producer cells at a neutral to slightly basic pH.

### Example 2—Production of Pseudotyped Lentiviral Vectors

This example sets forth an exemplary process for the production of pseudotyped lentiviral vectors.

HEK293 producer cells are grown in a various sized bioreactors. For example, cells are grown in either a 3 L or 200 L bioreactor (Single-use Bioreactor, EMD Millipore) such that they are an appropriate density for transfection, e.g., the cells are allowed to grow to a target cell density range of 2.5-3.5e⁶ cells/mL of media. Cells are then transfected with plasmids expressing viral vector proteins (gag/pol, rev) and a GFP transgene transfer plasmid. Envelope proteins are provided as plasmids expressing VSV-G or Nipah F protein and a CD8-retargeted Nipah G protein (see US 2019/0144885, incorporated by reference herein). The CD8-retargered Nipah G (NiV-G) protein contains an anti-CD8 scFv as a fusion with the exemplary NiV-G sequence GcΔ34 (set forth in SEQ ID NO:34), and the Nipah F (NiV-F) protein is the exemplary NiV-F sequence NivFdel22 (SEQ ID NO:33; or SEQ ID NO:38 without a signal sequence) as described in Example 1 above.

pH and DO are monitored by using probes inserted into the bioreactor. pH control is provided by sodium carbonate and CO2, and DO control is provided by O2, air, and nitrogen. For cells grown in 3 L bioreactors, pH is controlled from Day 0 and pH is controlled from the time of cell seeding when grown in bioreactors of 200 L. Cell growth and viability are monitored during production, with glucose and sodium butyrate supplementation on Day 2 (i.e., 24 hours post transfection).

Following viral vector production, the cell culture is harvested on Day 3 (i.e., 48 hours post transfection) via centrifuge to pellet the cells. The supernatant containing crude virus is collected for downstream processing.

### Example 3—Effect of pH and Dissolved Oxygen on Functional Titer of Pseudotyped Lentiviral Vectors

To determine the effect of pH and dissolved oxygen concentration (DO) on production of retargeted Nipah pseudotyped lentiviral vectors, a response surface experiment was designed as described in Example 1 and 2. An experiment was performed in which pH set points of 7.05, 7.20, and 7.35 with a deadband of 0.05 were tested, and DO ranged from 20-60%. In a complementary experiment, DO was maintained at 40% while pH was assessed at two setpoints: 7.15 with a 0.15 deadband and 7.20 with a 0.05 deadband.

Viral vectors were produced similar to methods described in Example 1 and 2, in which pH and DO were monitored by using probes inserted into the bioreactor. pH control was provided by sodium carbonate and CO₂, and DO control was provided by O2, air, and nitrogen. Cell growth and viability were monitored during production. Following viral vector production, the cell culture was centrifuged to pellet the cells and the supernatant containing crude virus was collected. The crude viral supernatant was tested for functional titer on SupT1 cells as described in Example 1.

In this experiment, no significant change from control (pH 7.2) was observed at the other pH set points tested. Further, no significant change from control (pH 7.2, 0.05 dead band) was observed with other pH control strategies including different pH dead bands. This result confirms control of improved production of lentiviral vectors pseudotyped with Paramyxovirus fusogens can be achieved by culturing producer cells at an approximately neutral or slightly basic pH.

### Example 4—Effect of pH Drift on Functional Titer of Pseudotyped Lentiviral Vectors

To determine the effect of using a control strategy with a dead band set to allow pH to drift over time, several such control strategies were assessed in an exemplary process for the production of pseudotyped lentiviral vectors. Table 4 below sets forth pH strategies that were assessed in this experiment.

TABLE 4
Exemplary conditions for Design Experiments

| Reactor | pH | DO | |
|---|---|---|---|
| R101 | 7.20 | 40 | 0.05 |
| R102 | 7.20 | 40 | 0.05 |
| R103 | 7.05 | 60 | 0.05 |
| R104 | 7.05 | 60 | 0.05 |
| R105 | 7.15 | 40 | 0.10 |
| R106 | 7.15 | 40 | 0.10 |
| R107 | 7.15 | 60 | 0.10 |
| R108 | 7.15 | 60 | 0.10 |

Viral vectors were produced similar to methods described in Example 1 and 2, in which pH and DO were monitored by using probes inserted into the bioreactor. pH control was provided by sodium carbonate and CO₂, and DO control was provided by O2, air, and nitrogen as set forth in Table 4 above. Cell growth and viability were also monitored during production. Following viral vector production, the cell culture was centrifuged to pellet the cells and the supernatant containing crude virus was collected. The crude viral supernatant was tested for functional titer on SupT1 cells as described in Example 1.

The results showed that the tested conditions exhibit similar functional titer across DO setpoints, pH setpoints, and pH deadbands. Mean functional titer as TU/mL for various exemplary conditions is shown in FIG. 3A, while mean infectivity as VLP/TU is shown for the exemplary conditions in FIG. 3B. As shown, functional titer and infectivity were slightly better with 60% DO, pH 7.15 and a dead band of 0.10.

This result confirms control of improved production of lentiviral vectors pseudotyped with Paramyxovirus fusogens can be achieved by culturing producer cells under conditions with a neutral or slightly basic pH across different DO set points and pH setpoints.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### SEQUENCES

| SEQ ID | Sequence | Annotation |
|---|---|---|
| 1 | MPAENKKVRFENTTSDKGKIPSKVIKSYYGTMDIKKINEGLLD | wild-type NiV-G protein |
| | SKILSAFNTVIALLGSIVIIVMNIMIIQNYTRSTDNQAVIKDALQ | |
| | GIQQQIKGLADKIGTEIGPKVSLIDTSSTITIPANIGLLGSKISQST | |
| | ASINENVNEKCKFTLPPLKIHECNISCPNPLPFREYRPQTEGVSN | |
| | LVGLPNNICLQKTSNQILKPKLISYTLPVVGQSGTCITDPLLAM | |
| | DEGYFAYSHLERIGSCSRGVSKQRIIGVGEVLDRGDEVPSLFM | |
| | TNVWTPPNPNTVYHCSAVYNNEFYYVLCAVSTVGDPILNSTY | |
| | WSGSLMMTRLAVKPKSNGGGYNQHQLALRSIEKGRYDKVMP | |
| | YGPSGIKQGDTLYFPAVGFLVRTEFKYNDSNCPITKCQYSKPE | |
| | NCRLSMGIRPNSHYILRSGLLKYNLSDGENPKVVFIEISDQRLSI | |
| | GSPSKIYDSLGQPVFYQASFSWDTMIKFGDVLTVNPLVVNWR | |
| | NNTVISRPGQSQCPRFNTCPEICWEGVYNDAFLIDRINWISAGV | |
| | FLDSNQTAENPVFTVFKDNEILYRAQLASEDTNAQKTITNCFL | |
| | LKNKIWCISLVEIYDTGDNVIRPKLFAVKIPEQCT | |
| 2 | PAENKKVRFENTTSDKGKIPSKVIKSYYGTMDIKKINEGLLDS | Nipah Virus G Protein |
| | KILSAFNTVIALLGSIVIIVMNIMIIQNYTRSTDNQAVIKDALQGI | (No Met) |
| | QQQIKGLADKIGTEIGPKVSLIDTSSTITIPANIGLLGSKISQSTA | |
| | SINENVNEKCKFTLPPLKIHECNISCPNPLPFREYRPQTEGVSNL | |
| | VGLPNNICLQKTSNQILKPKLISYTLPVVGQSGTCITDPLLAMD | |
| | EGYFAYSHLERIGSCSRGVSKQRIIGVGEVLDRGDEVPSLFMT | |
| | NVWTPPNPNTVYHCSAVYNNEFYYVLCAVSTVGDPILNSTYW | |
| | SGSLMMTRLAVKPKSNGGGYNQHQLALRSIEKGRYDKVMPY | |
| | GPSGIKQGDTLYFPAVGFLVRTEFKYNDSNCPITKCQYSKPEN | |
| | CRLSMGIRPNSHYILRSGLLKYNLSDGENPKVVFIEISDQRLSIG | |
| | SPSKIYDSLGQPVFYQASFSWDTMIKFGDVLTVNPLVVNWRN | |
| | NTVISRPGQSQCPRFNTCPEICWEGVYNDAFLIDRINWISAGVF | |
| | LDSNQTAENPVFTVFKDNEILYRAQLASEDTNAQKTITNCFLL | |
| | KNKIWCISLVEIYDTGDNVIRPKLFAVKIPEQCT | |
| 3 | MVVILDKRCYCNLLILILMISECSVGILHYEKLSKIGLVKGVTR | Nipah virus F Protein |
| | KYKIKSNPLTKDIVIKMIPNVSNMSQCTGSVMENYKTRLNGIL | |
| | TPIKGALEIYKNNTHDLVGDVRLAGVIMAGVAIGIATAAQITA | |
| | GVALYEAMKNADNINKLKSSIESTNEAVVKLQETAEKTVYVL | |
| | TALQDYINTNLVPTIDKISCKQTELSLDLALSKYLSDLLFVFGP | |
| | NLQDPVSNSMTIQAISQAFGGNYETLLRTLGYATEDFDDLLES | |
| | DSITGQIIYVDLSSYYIIVRVYFPILTEIQQAYIQELLPVSFNNDN | |
| | SEWISIVPNFILVRNTLISNIEIGFCLITKRSVICNQDYATPMTNN | |
| | MRECLTGSTEKCPRELVVSSHVPRFALSNGVLFANCISVTCQC | |
| | QTTGRAISQSGEQTLLMIDNTTCPTAVLGNVIISLGKYLGSVNY | |
| | NSEGIAIGPPVFTDKVDISSQISSMNQSLQQSKDYIKEAQRLLD | |
| | TVNPSLISMLSMIILYVLSIASLCIGLITFISFIIVEKKRNTYSRLE | |
| | DRRVRPTSSGDLYYIGT | |
| 4 | ILHYEKLSKIGLVKGVTRKYKIKSNPLTKDIVIKMIPNVSNMSQ | Nipah virus F Protein, |
| | CTGSVMENYKTRLNGILTPIKGALEIYKNNTHDLVGDVRLAG | without signal sequence |
| | VIMAGVAIGIATAAQITAGVALYEAMKNADNINKLKSSIESTN | |
| | EAVVKLQETAEKTVYVLTALQDYINTNLVPTIDKISCKQTELS | |
| | LDLALSKYLSDLLFVFGPNLQDPVSNSMTIQAISQAFGGNYET | |
| | LLRTLGYATEDFDDLLESDSITGQIIYVDLSSYYIIVRVYFPILTE | |
| | IQQAYIQELLPVSFNNDNSEWISIVPNFILVRNTLISNIEIGFCLIT | |
| | KRSVICNQDYATPMTNNMRECLTGSTEKCPRELVVSSHVPRF | |
| | ALSNGVLFANCISVTCQCQTTGRAISQSGEQTLLMIDNTTCPTA | |
| | VLGNVIISLGKYLGSVNYNSEGIAIGPPVFTDKVDISSQISSMNQ | |
| | SLQQSKDYIKEAQRLLDTVNPSLISMLSMIILYVLSIASLCIGLIT | |
| | FISFIIVEKKRNTYSRLEDRRVRPTSSGDLYYIGT | |
| 5 | MGPAENKKVR FENTTSDKGK IPSKVIKSYY | NiVG protein attachment |
| | GTMDIKKINE GLLDSKILSA FNTVIALLGS IVIIVMNIMI | glycoprotein (602 aa) |
| | IQNYTRSTDN QAVIKDALQG IQQQIKGLAD | |
| | KIGTEIGPKV SLIDTSSTIT IPANIGLLGS KISQSTASIN | |
| | ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRENTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QC | |
| 6 | MMADSKLVSLNNNLSGKIKDQGKVIKNYYGTMDIKKIN | Hendra Virus G Protein |
| | DGLLDSKILGAFNTVIALLGSIIIIVMNIMIIQNYTRTTDNQ | |
| | ALIKESLQSVQQQIKALTDKIGTEIGPKVSLIDTSSTITIPA | |
| | NIGLLGSKISQSTSSINENVNDKCKFTLPPLKIHECNISCP | |
| | NPLPFREYRPISQGVSDLVGLPNQICLQKTTSTILKPRLIS | |
| | YTLPINTREGVCITDPLLAVDNGFFAYSHLEKIGSCTRGI | |
| | AKQRIIGVGEVLDRGDKVPSMFMTNVWTPPNPSTIHHCS | |
| | STYHEDFYYTLCAVSHVGDPILNSTSWTESLSLIRLAVRP | |
| | KSDSGDYNQKYIAITKVERGKYDKVMPYGPSGIKQGDT | |
| | LYFPAVGFLPRTEFQYNDSNCPIIHCKYSKAENCRLSMG | |
| | VNSKSHYILRSGLLKYNLSLGGDIILQFIEIADNRLTIGSPS | |
| | KIYNSLGQPVFYQASYSWDTMIKLGDVDTVDPLRVQW | |
| | RNNSVISRPGQSQCPRFNVCPEVCWEGTYNDAFLIDRLN | |
| | WVSAGVYLNSNQTAENPVFAVFKDNEILYQVPLAEDDT | |
| | NAQKTITDCFLLENVIWCISLVEIYDTGDSVIRPKLFAVKI | |
| | PAQCSES | |
| 7 | MADSKLVSLNNNLSGKIKDQGKVIKNYYGTMDIKKIND | Hendra Virus G Protein |
| | GLLDSKILGAFNTVIALLGSIIIIVMNIMIIQNYTRTTDNQA | without Met |
| | LIKESLQSVQQQIKALTDKIGTEIGPKVSLIDTSSTITIPANI | |
| | GLLGSKISQSTSSINENVNDKCKFTLPPLKIHECNISCPNP | |
| | LPFREYRPISQGVSDLVGLPNQICLQKTTSTILKPRLISYT | |
| | LPINTREGVCITDPLLAVDNGFFAYSHLEKIGSCTRGIAK | |
| | QRIIGVGEVLDRGDKVPSMFMTNVWTPPNPSTIHHCSST | |
| | YHEDFYYTLCAVSHVGDPILNSTSWTESLSLIRLAVRPKS | |
| | DSGDYNQKYIAITKVERGKYDKVMPYGPSGIKQGDTLY | |
| | FPAVGFLPRTEFQYNDSNCPIIHCKYSKAENCRLSMGVN | |
| | SKSHYILRSGLLKYNLSLGGDIILQFIEIADNRLTIGSPSKI | |
| | YNSLGQPVFYQASYSWDTMIKLGDVDTVDPLRVQWRN | |
| | NSVISRPGQSQCPRFNVCPEVCWEGTYNDAFLIDRLNW | |
| | VSAGVYLNSNQTAENPVFAVFKDNEILYQVPLAEDDTN | |
| | AQKTITDCFLLENVIWCISLVEIYDTGDSVIRPKLFAVKIP | |
| | AQCSES | |
| 8 | MPAENKKVRFENTTSDKGKIPSKVIKSYYGTMDIKKINE | Nipah Virus G Protein |
| | GLLDSKILSAFNTVIALLGSIVIIVMNIMIIQNYTRSTDNQ | |
| | AVIKDALQGIQQQIKGLADKIGTEIGPKVSLIDTSSTITIPA | |
| | NIGLLGSKISQSTASINENVNEKCKFTLPPLKIHECNISCP | |
| | NPLPFREYRPQTEGVSNLVGLPNNICLQKTSNQILKPKLI | |
| | SYTLPVVGQSGTCITDPLLAMDEGYFAYSHLERIGSCSR | |
| | GVSKQRIIGVGEVLDRGDEVPSLFMTNVWTPPNPNTVY | |
| | HCSAVYNNEFYYVLCAVSTVGDPILNSTYWSGSLMMTR | |
| | LAVKPKSNGGGYNQHQLALRSIEKGRYDKVMPYGPSGI | |
| | KQGDTLYFPAVGFLVRTEFKYNDSNCPITKCQYSKPENC | |
| | RLSMGIRPNSHYILRSGLLKYNLSDGENPKVVFIEISDQR | |
| | LSIGSPSKIYDSLGQPVFYQASFSWDTMIKFGDVLTVNPL | |
| | VVNWRNNTVISRPGQSQCPRFNTCPEICWEGVYNDAFLI | |
| | DRINWISAGVFLDSNQTAENPVFTVFKDNEILYRAQLAS | |
| | EDTNAQKTITNCFLLKNKIWCISLVEIYDTGDNVIRPKLF | |
| | AVKIPEQCT | |
| 9 | PAENKKVRFENTTSDKGKIPSKVIKSYYGTMDIKKINEG | Nipah Virus G Protein |
| | LLDSKILSAFNTVIALLGSIVIIVMNIMIIQNYTRSTDNQA | (No Met) |
| | VIKDALQGIQQQIKGLADKIGTEIGPKVSLIDTSSTITIPAN | |
| | IGLLGSKISQSTASINENVNEKCKFTLPPLKIHECNISCPNP | |
| | LPFREYRPQTEGVSNLVGLPNNICLQKTSNQILKPKLISY | |
| | TLPVVGQSGTCITDPLLAMDEGYFAYSHLERIGSCSRGV | |
| | SKQRIIGVGEVLDRGDEVPSLFMTNVWTPPNPNTVYHCS | |
| | AVYNNEFYYVLCAVSTVGDPILNSTYWSGSLMMTRLA | |
| | VKPKSNGGGYNQHQLALRSIEKGRYDKVMPYGPSGIKQ | |
| | GDTLYFPAVGFLVRTEFKYNDSNCPITKCQYSKPENCRL | |
| | SMGIRPNSHYILRSGLLKYNLSDGENPKVVFIEISDQRLSI | |
| | GSPSKIYDSLGQPVFYQASFSWDTMIKFGDVLTVNPLVV | |
| | NWRNNTVISRPGQSQCPRFNTCPEICWEGVYNDAFLIDR | |
| | INWISAGVFLDSNQTAENPVFTVFKDNEILYRAQLASED | |
| | TNAQKTITNCFLLKNKIWCISLVEIYDTGDNVIRPKLFAV | |
| | KIPEQCT | |
| 10 | MLSQLQKNYLDNSNQQGDKMNNPDKKLSVNFNPLELD | Cedar Virus G Protein |
| | KGQKDLNKSYYVKNKNYNVSNLLNESLHDIKFCIYCIFS | |
| | LLIIITIINIITISIVITRLKVHEENNGMESPNLQSIQDSLSSL | |
| | TNMINTEITPRIGILVTATSVTLSSSINYVGTKTNQLVNEL | |
| | KDYITKSCGFKVPELKLHECNISCADPKISKSAMYSTNA | |
| | YAELAGPPKIFCKSVSKDPDFRLKQIDYVIPVQQDRSICM | |
| | NNPLLDISDGFFTYIHYEGINSCKKSDSFKVLLSHGEIVD | |
| | RGDYRPSLYLLSSHYHPYSMQVINCVPVTCNQSSFVFCH | |
| | ISNNTKTLDNSDYSSDEYYITYFNGIDRPKTKKIPINNMT | |
| | ADNRYIHFTFSGGGGVCLGEEFIIPVTTVINTDVFTHDYC | |
| | ESFNCSVQTGKSLKEICSESLRSPTNSSRYNLNGIMIISQN | |
| | NMTDFKIQLNGITYNKLSFGSPGRLSKTLGQVLYYQSSM | |
| | SWDTYLKAGFVEKWKPFTPNWMNNTVISRPNQGNCPR | |
| | YHKCPEICYGGTYNDIAPLDLGKDMYVSVILDSDQLAE | |
| | NPEITVFNSTTILYKERVSKDELNTRSTTTSCFLFLDEPW | |
| | CISVLETNRFNGKSIRPEIYSYKIPKYC | |
| 11 | LSQLQKNYLDNSNQQGDKMNNPDKKLSVNFNPLELDK | Cedar Virus G Protein |
| | GQKDLNKSYYVKNKNYNVSNLLNESLHDIKFCIYCIFSL | (No Met) |
| | LIIITIINIITISIVITRLKVHEENNGMESPNLQSIQDSLSSLT | |
| | NMINTEITPRIGILVTATSVTLSSSINYVGTKTNQLVNELK | |
| | DYITKSCGFKVPELKLHECNISCADPKISKSAMYSTNAY | |
| | AELAGPPKIFCKSVSKDPDFRLKQIDYVIPVQQDRSICMN | |
| | NPLLDISDGFFTYIHYEGINSCKKSDSFKVLLSHGEIVDR | |
| | GDYRPSLYLLSSHYHPYSMQVINCVPVTCNQSSFVFCHI | |
| | SNNTKTLDNSDYSSDEYYITYFNGIDRPKTKKIPINNMTA | |
| | DNRYIHFTFSGGGGVCLGEEFIIPVTTVINTDVFTHDYCE | |
| | SFNCSVQTGKSLKEICSESLRSPTNSSRYNLNGIMIISQNN | |
| | MTDFKIQLNGITYNKLSFGSPGRLSKTLGQVLYYQSSMS | |
| | WDTYLKAGFVEKWKPFTPNWMNNTVISRPNQGNCPRY | |
| | HKCPEICYGGTYNDIAPLDLGKDMYVSVILDSDQLAENP | |
| | EITVFNSTTILYKERVSKDELNTRSTTTSCFLFLDEPWCIS | |
| | VLETNRFNGKSIRPEIYSYKIPKYC | |
| 12 | MPQKTVEFINMNSPLERGVSTLSDKKTLNQSKITKQGYF | Bat Paramyxovirus G |
| | GLGSHSERNWKKQKNQNDHYMTVSTMILEILVVLGIMF | Protein |
| | NLIVLTMVYYQNDNINQRMAELTSNITVLNLNLNQLTN | |
| | KIQREIIPRITLIDTATTITIPSAITYILATLTTRISELLPSINQ | |
| | KCEFKTPTLVLNDCRINCTPPLNPSDGVKMSSLATNLVA | |
| | HGPSPCRNFSSVPTIYYYRIPGLYNRTALDERCILNPRLTI | |
| | SSTKFAYVHSEYDKNCTRGFKYYELMTFGEILEGPEKEP | |
| | RMFSRSFYSPTNAVNYHSCTPIVTVNEGYFLCLECTSSDP | |
| | LYKANLSNSTFHLVILRHNKDEKIVSMPSFNLSTDQEYV | |
| | QIIPAEGGGTAESGNLYFPCIGRLLHKRVTHPLCKKSNCS | |
| | RTDDESCLKSYYNQGSPQHQVVNCLIRIRNAQRDNPTW | |
| | DVITVDLTNTYPGSRSRIFGSFSKPMLYQSSVSWHTLLQ | |
| | VAEITDLDKYQLDWLDTPYISRPGGSECPFGNYCPTVCW | |
| | EGTYNDVYSLTPNNDLFVTVYLKSEQVAENPYFAIFSRD | |
| | QILKEFPLDAWISSARTTTISCFMFNNEIWCIAALEITRLN | |
| | DDIIRPIYYSFWLPTDCRTPYPHTGKMTRVPLRSTYNY | |
| 13 | PQKTVEFINMNSPLERGVSTLSDKKTLNQSKITKQGYFG | Bat Paramyxovirus G |
| | LGSHSERNWKKQKNQNDHYMTVSTMILEILVVLGIMEN | Protein (No Met) |
| | LIVLTMVYYQNDNINQRMAELTSNITVLNLNLNQLTNKI | |
| | QREIIPRITLIDTATTITIPSAITYILATLTTRISELLPSINQKC | |
| | EFKTPTLVLNDCRINCTPPLNPSDGVKMSSLATNLVAHG | |
| | PSPCRNFSSVPTIYYYRIPGLYNRTALDERCILNPRLTISST | |
| | KFAYVHSEYDKNCTRGFKYYELMTFGEILEGPEKEPRM | |
| | FSRSFYSPTNAVNYHSCTPIVTVNEGYFLCLECTSSDPLY | |
| | KANLSNSTFHLVILRHNKDEKIVSMPSFNLSTDQEYVQII | |
| | PAEGGGTAESGNLYFPCIGRLLHKRVTHPLCKKSNCSRT | |
| | DDESCLKSYYNQGSPQHQVVNCLIRIRNAQRDNPTWDV | |
| | ITVDLTNTYPGSRSRIFGSFSKPMLYQSSVSWHTLLQVAE | |
| | ITDLDKYQLDWLDTPYISRPGGSECPFGNYCPTVCWEGT | |
| | YNDVYSLTPNNDLFVTVYLKSEQVAENPYFAIFSRDQIL | |
| | KEFPLDAWISSARTTTISCFMFNNEIWCIAALEITRLNDDI | |
| | IRPIYYSFWLPTDCRTPYPHTGKMTRVPLRSTYNY | |
| 14 | MATNRDNTITSAEVSQEDKVKKYYGVETAEKVADSISG | Mojiang virus, Tongguan |
| | NKVFILMNTLLILTGAIITITLNITNLTAAKSQQNMLKIIQ | 1 G Protein |
| | DDVNAKLEMFVNLDQLVKGEIKPKVSLINTAVSVSIPGQ | |
| | ISNLQTKFLQKYVYLEESITKQCTCNPLSGIFPTSGPTYPP | |
| | TDKPDDDTTDDDKVDTTIKPIEYPKPDGCNRTGDHFTM | |
| | EPGANFYTVPNLGPASSNSDECYTNPSFSIGSSIYMFSQEI | |
| | RKTDCTAGEILSIQIVLGRIVDKGQQGPQASPLLVWAVP | |
| | NPKIINSCAVAAGDEMGWVLCSVTLTAASGEPIPHMFD | |
| | GFWLYKLEPDTEVVSYRITGYAYLLDKQYDSVFIGKGG | |
| | GIQKGNDLYFQMYGLSRNRQSFKALCEHGSCLGTGGGG | |
| | YQVLCDRAVMSFGSEESLITNAYLKVNDLASGKPVIIGQ | |
| | TFPPSDSYKGSNGRMYTIGDKYGLYLAPSSWNRYLRFGI | |
| | TPDISVRSTTWLKSQDPIMKILSTCTNTDRDMCPEICNTR | |
| | GYQDIFPLSEDSEYYTYIGITPNNGGTKNFVAVRDSDGHI | |
| | ASIDILQNYYSITSATISCFMYKDEIWCIAITEGKKQKDNP | |
| | QRIYAHSYKIRQMCYNMKSATVTVGNAKNITIRRY | |
| 15 | ATNRDNTITSAEVSQEDKVKKYYGVETAEKVADSISGN | Mojiang virus, Tongguan |
| | KVFILMNTLLILTGAIITITLNITNLTAAKSQQNMLKIIQD | 1 G (No Met) |
| | DVNAKLEMFVNLDQLVKGEIKPKVSLINTAVSVSIPGQIS | |
| | NLQTKFLQKYVYLEESITKQCTCNPLSGIFPTSGPTYPPT | |
| | DKPDDDTTDDDKVDTTIKPIEYPKPDGCNRTGDHFTMEP | |
| | GANFYTVPNLGPASSNSDECYTNPSFSIGSSIYMFSQEIR | |
| | KTDCTAGEILSIQIVLGRIVDKGQQGPQASPLLVWAVPN | |
| | PKIINSCAVAAGDEMGWVLCSVTLTAASGEPIPHMFDGF | |
| | WLYKLEPDTEVVSYRITGYAYLLDKQYDSVFIGKGGGIQ | |
| | KGNDLYFQMYGLSRNRQSFKALCEHGSCLGTGGGGYQ | |
| | VLCDRAVMSFGSEESLITNAYLKVNDLASGKPVIIGQTFP | |
| | PSDSYKGSNGRMYTIGDKYGLYLAPSSWNRYLRFGITPD | |
| | ISVRSTTWLKSQDPIMKILSTCTNTDRDMCPEICNTRGYQ | |
| | DIFPLSEDSEYYTYIGITPNNGGTKNFVAVRDSDGHIASI | |
| | DILQNYYSITSATISCFMYKDEIWCIAITEGKKQKDNPQR | |
| | IYAHSYKIRQMCYNMKSATVTVGNAKNITIRRY | |
| 16 | MATQEVRLKCLLCGIIVLVLSLEGLGILHYEKLSKIGLVK | Hendra virus F Protein |
| | GITRKYKIKSNPLTKDIVIKMIPNVSNVSKCTGTVMENY | |
| | KSRLTGILSPIKGAIELYNNNTHDLVGDVKLAGVVMAGI | |
| | AIGIATAAQITAGVALYEAMKNADNINKLKSSIESTNEA | |
| | VVKLQETAEKTVYVLTALQDYINTNLVPTIDQISCKQTE | |
| | LALDLALSKYLSDLLFVFGPNLQDPVSNSMTIQAISQAF | |
| | GGNYETLLRTLGYATEDFDDLLESDSIAGQIVYVDLSSY | |
| | YIIVRVYFPILTEIQQAYVQELLPVSFNNDNSEWISIVPNF | |
| | VLIRNTLISNIEVKYCLITKKSVICNQDYATPMTASVREC | |
| | LTGSTDKCPRELVVSSHVPRFALSGGVLFANCISVTCQC | |
| | QTTGRAISQSGEQTLLMIDNTTCTTVVLGNIIISLGKYLGS | |
| | INYNSESIAVGPPVYTDKVDISSQISSMNQSLQQSKDYIK | |
| | EAQKILDTVNPSLISMLSMIILYVLSIAALCIGLITFISFVIV | |
| | EKKRGNYSRLDDRQVRPVSNGDLYYIGT | |
| 17 | ILHYEKLSKIGLVKGITRKYKIKSNPLTKDIVIKMIPNVSN | Hendra virus F Protein, |
| | VSKCTGTVMENYKSRLTGILSPIKGAIELYNNNTHDLVG | Without signal sequence |
| | DVKLAGVVMAGIAIGIATAAQITAGVALYEAMKNADNI | |
| | NKLKSSIESTNEAVVKLQETAEKTVYVLTALQDYINTNL | |
| | VPTIDQISCKQTELALDLALSKYLSDLLFVFGPNLQDPVS | |
| | NSMTIQAISQAFGGNYETLLRTLGYATEDFDDLLESDSIA | |
| | GQIVYVDLSSYYIIVRVYFPILTEIQQAYVQELLPVSENN | |
| | DNSEWISIVPNFVLIRNTLISNIEVKYCLITKKSVICNQDY | |
| | ATPMTASVRECLTGSTDKCPRELVVSSHVPRFALSGGVL | |
| | FANCISVTCQCQTTGRAISQSGEQTLLMIDNTTCTTVVL | |
| | GNIIISLGKYLGSINYNSESIAVGPPVYTDKVDISSQISSM | |
| | NQSLQQSKDYIKEAQKILDTVNPSLISMLSMIILYVLSIAA | |
| | LCIGLITFISFVIVEKKRGNYSRLDDRQVRPVSNGDLYYI | |
| | GT | |
| 18 | MVVILDKRCYCNLLILILMISECSVGILHYEKLSKIGLVK | Nipah virus F Protein |
| | GVTRKYKIKSNPLTKDIVIKMIPNVSNMSQCTGSVMENY | |
| | KTRLNGILTPIKGALEIYKNNTHDLVGDVRLAGVIMAGV | |
| | AIGIATAAQITAGVALYEAMKNADNINKLKSSIESTNEA | |
| | VVKLQETAEKTVYVLTALQDYINTNLVPTIDKISCKQTE | |
| | LSLDLALSKYLSDLLFVFGPNLQDPVSNSMTIQAISQAFG | |
| | GNYETLLRTLGYATEDFDDLLESDSITGQIIYVDLSSYYII | |
| | VRVYFPILTEIQQAYIQELLPVSFNNDNSEWISIVPNFILV | |
| | RNTLISNIEIGFCLITKRSVICNQDYATPMTNNMRECLTG | |
| | STEKCPRELVVSSHVPRFALSNGVLFANCISVTCQCQTT | |
| | GRAISQSGEQTLLMIDNTTCPTAVLGNVIISLGKYLGSVN | |
| | YNSEGIAIGPPVFTDKVDISSQISSMNQSLQQSKDYIKEA | |
| | QRLLDTVNPSLISMLSMIILYVLSIASLCIGLITFISFIIVEK | |
| | KRNTYSRLEDRRVRPTSSGDLYYIGT | |
| 19 | ILHYEKLSKIGLVKGVTRKYKIKSNPLTKDIVIKMIPNVS | Nipah virus F Protein, |
| | NMSQCTGSVMENYKTRLNGILTPIKGALEIYKNNTHDL | without signal sequence |
| | VGDVRLAGVIMAGVAIGIATAAQITAGVALYEAMKNAD | |
| | NINKLKSSIESTNEAVVKLQETAEKTVYVLTALQDYINT | |
| | NLVPTIDKISCKQTELSLDLALSKYLSDLLFVFGPNLQDP | |
| | VSNSMTIQAISQAFGGNYETLLRTLGYATEDFDDLLESD | |
| | SITGQIIYVDLSSYYIIVRVYFPILTEIQQAYIQELLPVSEN | |
| | NDNSEWISIVPNFILVRNTLISNIEIGFCLITKRSVICNQDY | |
| | ATPMTNNMRECLTGSTEKCPRELVVSSHVPRFALSNGV | |
| | LFANCISVTCQCQTTGRAISQSGEQTLLMIDNTTCPTAVL | |
| | GNVIISLGKYLGSVNYNSEGIAIGPPVFTDKVDISSQISSM | |
| | NQSLQQSKDYIKEAQRLLDTVNPSLISMLSMIILYVLSIA | |
| | SLCIGLITFISFIIVEKKRNTYSRLEDRRVRPTSSGDLYYIG | |
| | T | |
| 20 | MSNKRTTVLIIISYTLFYLNNAAIVGFDFDKLNKIGVVQG | Cedar Virus F Protein |
| | RVLNYKIKGDPMTKDLVLKFIPNIVNITECVREPLSRYNE | |
| | TVRRLLLPIHNMLGLYLNNTNAKMTGLMIAGVIMGGIAI | |
| | GIATAAQITAGFALYEAKKNTENIQKLTDSIMKTQDSIDK | |
| | LTDSVGTSILILNKLQTYINNQLVPNLELLSCRQNKIEFDL | |
| | MLTKYLVDLMTVIGPNINNPVNKDMTIQSLSLLFDGNY | |
| | DIMMSELGYTPQDFLDLIESKSITGQIIYVDMENLYVVIR | |
| | TYLPTLIEVPDAQIYEFNKITMSSNGGEYLSTIPNFILIRGN | |
| | YMSNIDVATCYMTKASVICNQDYSLPMSQNLRSCYQGE | |
| | TEYCPVEAVIASHSPRFALTNGVIFANCINTICRCQDNGK | |
| | TITQNINQFVSMIDNSTCNDVMVDKFTIKVGKYMGRKDI | |
| | NNINIQIGPQIIIDKVDLSNEINKMNQSLKDSIFYLREAKRI | |
| | LDSVNISLISPSVQLFLIIISVLSFIILLIIIVYLYCKSKHSYK | |
| | YNKFIDDPDYYNDYKRERINGKASKSNNIYYVGD | |
| 21 | TVLIIISYTLFYLNNAAIVGFDFDKLNKIGVVQGRVLNYK | Cedar Virus F Protein, |
| | IKGDPMTKDLVLKFIPNIVNITECVREPLSRYNETVRRLL | without signal sequence |
| | LPIHNMLGLYLNNTNAKMTGLMIAGVIMGGIAIGIATAA | |
| | QITAGFALYEAKKNTENIQKLTDSIMKTQDSIDKLTDSV | |
| | GTSILILNKLQTYINNQLVPNLELLSCRQNKIEFDLMLTK | |
| | YLVDLMTVIGPNINNPVNKDMTIQSLSLLFDGNYDIMMS | |
| | ELGYTPQDFLDLIESKSITGQIIYVDMENLYVVIRTYLPTL | |
| | IEVPDAQIYEFNKITMSSNGGEYLSTIPNFILIRGNYMSNI | |
| | DVATCYMTKASVICNQDYSLPMSQNLRSCYQGETEYCP | |
| | VEAVIASHSPRFALTNGVIFANCINTICRCQDNGKTITQNI | |
| | NQFVSMIDNSTCNDVMVDKFTIKVGKYMGRKDINNINI | |
| | QIGPQIIIDKVDLSNEINKMNQSLKDSIFYLREAKRILDSV | |
| | NISLISPSVQLFLIIISVLSFIILLIIIVYLYCKSKHSYKYNKFI | |
| | DDPDYYNDYKRERINGKASKSNNIYYVGD | |
| 22 | MALNKNMFSSLFLGYLLVYATTVQSSIHYDSLSKVGVIK | Mojiang virus, Tongguan |
| | GLTYNYKIKGSPSTKLMVVKLIPNIDSVKNCTQKQYDEY | 1 F Protein |
| | KNLVRKALEPVKMAIDTMLNNVKSGNNKYRFAGAIMA | |
| | GVALGVATAATVTAGIALHRSNENAQAIANMKSAIQNT | |
| | NEAVKQLQLANKQTLAVIDTIRGEINNNIIPVINQLSCDTI | |
| | GLSVGIRLTQYYSEIITAFGPALQNPVNTRITIQAISSVEN | |
| | GNFDELLKIMGYTSGDLYEILHSELIRGNIIDVDVDAGYI | |
| | ALEIEFPNLTLVPNAVVQELMPISYNIDGDEWVTLVPRF | |
| | VLTRTTLLSNIDTSRCTITDSSVICDNDYALPMSHELIGCL | |
| | QGDTSKCAREKVVSSYVPKFALSDGLVYANCLNTICRC | |
| | MDTDTPISQSLGATVSLLDNKRCSVYQVGDVLISVGSYL | |
| | GDGEYNADNVELGPPIVIDKIDIGNQLAGINQTLQEAED | |
| | YIEKSEEFLKGVNPSIITLGSMVVLYIFMILIAIVSVIALVL | |
| | SIKLTVKGNVVRQQFTYTQHVPSMENINYVSH | |
| 23 | IHYDSLSKVGVIKGLTYNYKIKGSPSTKLMVVKLIPNIDS | Mojiang virus, Tongguan |
| | VKNCTQKQYDEYKNLVRKALEPVKMAIDTMLNNVKSG | 1 F Protein, without |
| | NNKYRFAGAIMAGVALGVATAATVTAGIALHRSNENA | signal sequence |
| | QAIANMKSAIQNTNEAVKQLQLANKQTLAVIDTIRGEIN | |
| | NNIIPVINQLSCDTIGLSVGIRLTQYYSEIITAFGPALQNPV | |
| | NTRITIQAISSVENGNFDELLKIMGYTSGDLYEILHSELIR | |
| | GNIIDVDVDAGYIALEIEFPNLTLVPNAVVQELMPISYNI | |
| | DGDEWVTLVPRFVLTRTTLLSNIDTSRCTITDSSVICDND | |
| | YALPMSHELIGCLQGDTSKCAREKVVSSYVPKFALSDGL | |
| | VYANCLNTICRCMDTDTPISQSLGATVSLLDNKRCSVYQ | |
| | VGDVLISVGSYLGDGEYNADNVELGPPIVIDKIDIGNQLA | |
| | GINQTLQEAEDYIEKSEEFLKGVNPSIITLGSMVVLYIFMI | |
| | LIAIVSVIALVLSIKLTVKGNVVRQQFTYTQHVPSMENIN | |
| | YVSH | |
| 24 | MKKKTDNPTISKRGHNHSRGIKSRALLRETDNYSNGLIV | Bat Paramyxovirus F |
| | ENLVRNCHHPSKNNLNYTKTQKRDSTIPYRVEERKGHY | Protein |
| | PKIKHLIDKSYKHIKRGKRRNGHNGNIITIILLLILILKTQM | |
| | SEGAIHYETLSKIGLIKGITREYKVKGTPSSKDIVIKLIPNV | |
| | TGLNKCTNISMENYKEQLDKILIPINNIIELYANSTKSAPG | |
| | NARFAGVIIAGVALGVAAAAQITAGIALHEARQNAERIN | |
| | LLKDSISATNNAVAELQEATGGIVNVITGMQDYINTNLV | |
| | PQIDKLQCSQIKTALDISLSQYYSEILTVFGPNLQNPVTTS | |
| | MSIQAISQSFGGNIDLLLNLLGYTANDLLDLLESKSITGQI | |
| | TYINLEHYFMVIRVYYPIMTTISNAYVQELIKISFNVDGS | |
| | EWVSLVPSYILIRNSYLSNIDISECLITKNSVICRHDFAMP | |
| | MSYTLKECLTGDTEKCPREAVVTSYVPRFAISGGVIYAN | |
| | CLSTTCQCYQTGKVIAQDGSQTLMMIDNQTCSIVRIEEIL | |
| | ISTGKYLGSQEYNTMHVSVGNPVFTDKLDITSQISNINQS | |
| | IEQSKFYLDKSKAILDKINLNLIGSVPISILFIIAILSLILSIIT | |
| | FVIVMIIVRRYNKYTPLINSDPSSRRSTIQDVYIIPNPGEHS | |
| | IRSAARSIDRDRD | |
| 25 | SRALLRETDNYSNGLIVENLVRNCHHPSKNNLNYTKTQ | Bat Paramyxovirus F |
| | KRDSTIPYRVEERKGHYPKIKHLIDKSYKHIKRGKRRNG | Protein, without signal |
| | HNGNIITIILLLILILKTQMSEGAIHYETLSKIGLIKGITREY | sequence |
| | KVKGTPSSKDIVIKLIPNVTGLNKCTNISMENYKEQLDKI | |
| | LIPINNIIELYANSTKSAPGNARFAGVIIAGVALGVAAAA | |
| | QITAGIALHEARQNAERINLLKDSISATNNAVAELQEAT | |
| | GGIVNVITGMQDYINTNLVPQIDKLQCSQIKTALDISLSQ | |
| | YYSEILTVFGPNLQNPVTTSMSIQAISQSFGGNIDLLLNLL | |
| | GYTANDLLDLLESKSITGQITYINLEHYFMVIRVYYPIMT | |
| | TISNAYVQELIKISFNVDGSEWVSLVPSYILIRNSYLSNIDI | |
| | SECLITKNSVICRHDFAMPMSYTLKECLTGDTEKCPREA | |
| | VVTSYVPRFAISGGVIYANCLSTTCQCYQTGKVIAQDGS | |
| | QTLMMIDNQTCSIVRIEEILISTGKYLGSQEYNTMHVSVG | |
| | NPVFTDKLDITSQISNINQSIEQSKFYLDKSKAILDKINLN | |
| | LIGSVPISILFIIAILSLILSIITFVIVMIIVRRYNKYTPLINSD | |
| | PSSRRSTIQDVYIIPNPGEHSIRSAARSIDRDRD | |
| 26 | MVVILDKRCY CNLLILILMI SECSVG | signal sequence |
| 27 | ILHYEKLSKIGLVKGVTRKYKIKSNPLTKDIVIKMIPNVS | Nipah virus NiV-F F2 (aa |
| | NMSQCTGSVMENYKTRLNGILTPIKGALEIYKNNTHDL | 27-109) |
| | VGDVR | |
| 28 | LAGVIMAGVAIGIATAAQITAGVALYEAMKNADNINKL | Nipah virus NiV F F1 (aa |
| | KSSIESTNEAVVKLQETAEKTVYVLTALQDYINTNLVPTI | 110-546) |
| | DKISCKQTELSLDLALSKYLSDLLFVFGPNLQDPVSNSM | |
| | TIQAISQAFGGNYETLLRTLGYATEDFDDLLESDSITGQII | |
| | YVDLSSYYIIVRVYFPILTEIQQAYIQELLPVSFNNDNSE | |
| | WISIVPNFILVRNTLISNIEIGFCLITKRSVICNQDYATPMT | |
| | NNMRECLTGSTEKCPRELVVSSHVPRFALSNGVLFANCI | |
| | SVTCQCQTTGRAISQSGEQTLLMIDNTTCPTAVLGNVIIS | |
| | LGKYLGSVNYNSEGIAIGPPVFTDKVDISSQISSMNQSLQ | |
| | QSKDYIKEAQRLLDTVNPSLISMLSMIILYVLSIASLCIGLI | |
| | TFISFIIVEKKRNTYSRLEDRRVRPTSSGDLYYIGT | |
| 29 | MKVR FENTTSDKGK IPSKVIKSYY GTMDIKKINE | NiVG protein attachment |
| | GLLDSKILSA FNTVIALLGS IVIIVMNIMI IQNYTRSTDN | glycoprotein |
| | QAVIKDALQG IQQQIKGLAD KIGTEIGPKV SLIDTSSTIT | Truncated Δ5 |
| | IPANIGLLGS KISQSTASIN ENVNEKCKFT LPPLKIHECN | |
| | ISCPNPLPFR EYRPQTEGVS NLVGLPNNIC | |
| | LQKTSNQILK PKLISYTLPV VGQSGTCITD | |
| | PLLAMDEGYF AYSHLERIGS CSRGVSKQRI | |
| | IGVGEVLDRG DEVPSLFMTN VWTPPNPNTV | |
| | YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF | |
| | LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRFNTC | |
| | PEICWEGVYN DAFLIDRINW ISAGVFLDSN | |
| | QTAENPVFTV FKDNEILYRA QLASEDTNAQ | |
| | KTITNCFLLK NKIWCISLVE IYDTGDNVIR PKLFAVKIPE | |
| | QCT | |
| 30 | MSKVIKSYY GTMDIKKINE GLLDSKILSA FNTVIALLGS | NiVG protein attachment |
| | IVIIVMNIMI IQNYTRSTDN QAVIKDALQG | glycoprotein |
| | IQQQIKGLAD KIGTEIGPKV SLIDTSSTIT IPANIGLLGS | Truncated Δ20 |
| | KISQSTASIN ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRENTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QCT | |
| 31 | MSYY GTMDIKKINE GLLDSKILSA FNTVIALLGS | NiVG protein attachment |
| | IVIIVMNIMI IQNYTRSTDN QAVIKDALQG | glycoprotein |
| | IQQQIKGLAD KIGTEIGPKV SLIDTSSTIT IPANIGLLGS | Truncated Δ25 |
| | KISQSTASIN ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRENTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QCT | |
| 32 | ILHY EKLSKIGLVK GVTRKYKIKS NPLTKDIVIK | Truncated NiV fusion |
| | MIPNVSNMSQ CTGSVMENYK TRLNGILTPI | glycoprotein (FcDelta22) |
| | KGALEIYKNN THDLVGDVRL AGVIMAGVAI | at cytoplasmic tail |
| | GIATAAQITA GVALYEAMKN ADNINKLKSS | (without signal sequence) |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 33 | MVVILDKRCY CNLLILILMI SECSVGILHY | Truncated NiV fusion |
| | EKLSKIGLVK GVTRKYKIKS NPLTKDIVIK | glycoprotein (FcDelta22) |
| | MIPNVSNMSQ CTGSVMENYK TRLNGILTPI | at cytoplasmic tail |
| | KGALEIYKNN THDLVGDVRL AGVIMAGVAI | (with signal sequence) |
| | GIATAAQITA GVALYEAMKN ADNINKLKSS | |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 34 | MKKINEGLLDSKILSA FNTVIALLGS IVIIVMNIMI | NiVG protein attachment |
| | IQNYTRSTDN QAVIKDALQG IQQQIKGLAD | glycoprotein |
| | KIGTEIGPKV SLIDTSSTIT IPANIGLLGS KISQSTASIN | Truncated and mutated |
| | ENVNEKCKFT LPPLKIHECN ISCPNPLPFR | (E501 A, W504A, |
| | EYRPQTEGVS NLVGLPNNIC LQKTSNQILK | Q530A, E533A) NiV G |
| | PKLISYTLPV VGQSGTCITD PLLAMDEGYF | protein (Gc Δ 34) |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRENTC PAICAEGVYN | |
| | DAFLIDRINW ISAGVFLDSN ATAANPVFTV | |
| | FKDNEILYRA QLASEDTNAQ KTITNCFLLK | |
| | NKIWCISLVE IYDTGDNVIR PKLFAVKIPE QCT | |
| 35 | KKINEGLLDSKILSA FNTVIALLGS IVIIVMNIMI | NiVG protein attachment |
| | IQNYTRSTDN QAVIKDALQG IQQQIKGLAD | glycoprotein |
| | KIGTEIGPKV SLIDTSSTIT IPANIGLLGS KISQSTASIN | Truncated and mutated |
| | ENVNEKCKFT LPPLKIHECN ISCPNPLPFR | (E501 A, W504A, |
| | EYRPQTEGVS NLVGLPNNIC LQKTSNQILK | Q530A, E533A) NiV G |
| | PKLISYTLPV VGQSGTCITD PLLAMDEGYF | protein (Gc Δ 34) |
| | AYSHLERIGS CSRGVSKQRI IGVGEVLDRG | Without N-terminal |
| | DEVPSLFMTN VWTPPNPNTV YHCSAVYNNE | methionine |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PAICAEGVYN | |
| | DAFLIDRINW ISAGVFLDSN ATAANPVFTV | |
| | FKDNEILYRA QLASEDTNAQ KTITNCFLLK | |
| | NKIWCISLVE IYDTGDNVIR PKLFAVKIPE QCT | |
| 36 | MVVILDKRCY CNLLILILMI SECSVGILHY | Truncated NiV fusion |
| | EKLSKIGLVK GVTRKYKIKS NPLTKDIVIK | glycoprotein (FcDelta22) |
| | MIPNVSNMSQ CTGSVMENYK TRLNGILTPI | at cytoplasmic tail |
| | KGALEIYKNN THDLVGDVRL AGVIMAGVAI | (with signal sequence) |
| | GIATAAQITA GVALYEAMKN ADNINKLKSS | |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 37 | ILHY EKLSKIGLVK GVTRKYKIKS NPLTKDIVIK | Nipah virus NiV-F F0 |
| | MIPNVSNMSQ CTGSVMENYK TRLNGILTPI | T234 truncation (aa 525- |
| | KGALEIYKNN THDLVGDVRL AGVIMAGVAI | 544) |
| | GIATAAQITA GVALYEAMKN ADNINKLKSS | |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 38 | ILHY EKLSKIGLVK GVTRKYKIKS NPLTKDIVIK | Truncated mature NiV |
| | MIPNVSNMSQ CTGSVMENYK TRLNGILTPI | fusion glycoprotein |
| | KGALEIYKNN THDLVGDVRL AGVIMAGVAI | (FcDelta22) at |
| | GIATAAQITA GVALYEAMKN ADNINKLKSS | cytoplasmic tail |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 39 | FNTVIALLGS IVIIVMNIMI IQNYTRSTDN | NivG protein attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | Without cytoplasmic tail |
| | ISCPNPLPFR EYRPQTEGVS NLVGLPNNIC | Uniprot Q9IH62 |
| | LQKTSNQILK PKLISYTLPV VGQSGTCITD | |
| | PLLAMDEGYF AYSHLERIGS CSRGVSKQRI | |
| | IGVGEVLDRG DEVPSLFMTN VWTPPNPNTV | |
| | YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF | |
| | LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRENTC | |
| | PEICWEGVYN DAFLIDRINW ISAGVFLDSN | |
| | QTAENPVFTV FKDNEILYRA QLASEDTNAQ | |
| | KTITNCFLLK NKIWCISLVE IYDTGDNVIR PKLFAVKIPE | |
| | QC | |
| 40 | MMADSKLVSL NNNLSGKIKD QGKVIKNYYG | Hendra virus G protein |
| | TMDIKKINDG LLDSKILGAF | Uniprot O89343 |
| | NTVIALLGSI IIIVMNIMII QNYTRTTDNQ ALIKESLQSV | |
| | QQQIKALTDK IGTEIGPKVS LIDTSSTITI PANIGLLGSK | |
| | ISQSTSSINE NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD | |
| | KVPSMFMTNV WTPPNPSTIH HCSSTYHEDF | |
| | YYTLCAVSHV | |
| | GDPILNSTSW TESLSLIRLA VRPKSDSGDY | |
| | NQKYIAITKV ERGKYDKVMP | |
| | YGPSGIKQGD TLYFPAVGFL PRTEFQYNDS | |
| | NCPIIHCKYS KAENCRLSMG | |
| | VNSKSHYILR SGLLKYNLSL GGDIILQFIE IADNRLTIGS | |
| | PSKIYNSLGQ PVFYQASYSW DTMIKLGDVD | |
| | TVDPLRVQWR NNSVISRPGQ SQCPRFNVCP | |
| | EVCWEGTYND AFLIDRLNWV SAGVYLNSNQ | |
| | TAENPVFAVF KDNEILYQVP LAEDDTNAQK | |
| | TITDCFLLEN VIWCISLVEI YDTGDSVIRP KLFAVKIPAQ | |
| | CSES | |
| 41 | MADSKLVSL NNNLSGKIKD QGKVIKNYYG | Hendra virus G protein |
| | TMDIKKINDG LLDSKILGAF | Uniprot O89343 Without |
| | NTVIALLGSI IIIVMNIMII QNYTRTTDNQ ALIKESLQSV | N-terminal methionine |
| | QQQIKALTDK IGTEIGPKVS LIDTSSTITI PANIGLLGSK | |
| | ISQSTSSINE NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD | |
| | KVPSMFMTNV WTPPNPSTIH HCSSTYHEDF | |
| | YYTLCAVSHV | |
| | GDPILNSTSW TESLSLIRLA VRPKSDSGDY | |
| | NQKYIAITKV ERGKYDKVMP | |
| | YGPSGIKQGD TLYFPAVGFL PRTEFQYNDS | |
| | NCPIIHCKYS KAENCRLSMG | |
| | VNSKSHYILR SGLLKYNLSL GGDIILQFIE IADNRLTIGS | |
| | PSKIYNSLGQ PVFYQASYSW DTMIKLGDVD | |
| | TVDPLRVQWR NNSVISRPGQ SQCPRFNVCP | |
| | EVCWEGTYND AFLIDRLNWV SAGVYLNSNQ | |
| | TAENPVFAVF KDNEILYQVP LAEDDTNAQK | |
| | TITDCFLLEN VIWCISLVEI YDTGDSVIRP KLFAVKIPAQ | |
| | CSES | |
| 42 | FNTVIALLGSI IIIVMNIMII QNYTRTTDNQ ALIKESLQSV | Hendra virus G protein |
| | QQQIKALTDK | Uniprot O89343 |
| | IGTEIGPKVS LIDTSSTITI PANIGLLGSK ISQSTSSINE | Without cytoplasmic tail |
| | NVNDKCKFTL | |
| | PPLKIHECNI SCPNPLPFRE YRPISQGVSD LVGLPNQICL | |
| | QKTTSTILKP | |
| | RLISYTLPIN TREGVCITDP LLAVDNGFFA YSHLEKIGSC | |
| | TRGIAKQRII | |
| | GVGEVLDRGD KVPSMFMTNV WTPPNPSTIH | |
| | HCSSTYHEDF YYTLCAVSHV | |
| | GDPILNSTSW TESLSLIRLA VRPKSDSGDY | |
| | NQKYIAITKV ERGKYDKVMP | |
| | YGPSGIKQGD TLYFPAVGFL PRTEFQYNDS | |
| | NCPIIHCKYS KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR | |
| | NNSVISRPGQ SQCPRFNVCP EVCWEGTYND | |
| | AFLIDRLNWV SAGVYLNSNQ TAENPVFAVF | |
| | KDNEILYQVP LAEDDTNAQK TITDCFLLEN | |
| | VIWCISLVEI YDTGDSVIRP KLFAVKIPAQ CSES | |
| 44 | MGPAENKKVR FENTTSDKGK IPSKVIKSYY | NiVG protein attachment |
| | GTMDIKKINE GLLDSKILSA FNTVIALLGS IVIIVMNIMI | glycoprotein (602 aa) |
| | IQNYTRSTDN QAVIKDALQG IQQQIKGLAD | |
| | KIGTEIGPKV SLIDTSSTIT IPANIGLLGS KISQSTASIN | |
| | ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRENTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QC | |
| 45 | (GₘS)ₙ | peptide linker |
| 46 | QIPRDRLSNIGVIVDEGKSLKIAGSHESRYIVLSLVP | Sendai F protein |
| | GVDFENGCGTAQVIQYKSLLNRLLIPLRDALDLQEA | |
| | LITVINDTTQNAGAPQSRFFGAVIGTIALGVATSAQI | |
| | TAGIALAEAREAKRDIALIKESMTKTHKSIELLQNA | |
| | VGEQILALKTLQDFVNDEIKPAISELGCETAALRLGI | |
| | KLTQHYSELLTAFGSNFGTIGEKSLTLQALSSLYSA | |
| | NITEIMTTIKTGQSNIYDVIYTEQIKGTVIDVDLERY | |
| | MVTLSVKIPILSEVPGVLIHKASSISYNIDGEEWYVT | |
| | VPSHILSRASFLGGADITDCVESRLTYICPRDPAQLIP | |
| | DSQQKCILGDTTRCPVTKVVDSLIPKFAFVNGGVV | |
| | ANCIASTCTCGTGRRPISQDRSKGVVFLTHDNCGLI | |
| | GVNGVELYANRRGHDATWGVQNLTVGPAIAIRPID | |
| | ISLNLADATNFLQDSKAE | |
| | LEKARKILSEVGRWYNSRETVITIIVVMVVILVVIIVI | |
| | IIVLYRLRRSMLMGNPDDRIPRDTYTLEPKIRHMYT | |
| | NGGFDAMAEKR | |
| 47 | MDGDRGKRDSYWSTSPSGSTTKPASGWERSSKADTWL | Sendai HN protein |
| | LILSFTQWALSIATVIICIIISARQGYSMKEYSMTVEALNM | |
| | SSREVKESLTSLIRQEVIARAVNIQSSVQTGIPVLLNKNSR | |
| | DVIQMIDKSCSRQELTQHCESTIAVHHADGIAPLEPHSF | |
| | WRCPVGEPYLSSDPEISLLPGPSLLSGSTTISGCVRLPSLSI | |
| | GEAIYAYSSNLITQGCADIGKSYQVLQLGYISLNSDMFP | |
| | DLNPVVSHTYDINDNRKSCSVVATGTRGYQLCSMPTVD | |
| | ERTDYSSDGIEDLVLDVLDLKGRTKSHRYRNSEVDLDH | |
| | PFSALYPSVGNGIATEGSLIFLGYGGLTTPLQGDTKCRTQ | |
| | GCQQVSQDTCNEALKITWLGGKQVVSVIIQVNDYLSER | |
| | PKIRVTTIPITQNYLGAEGRLLKLGDRVYIYTRSSGWHSQ | |
| | LQIGVLDVSHPLTINWTPHEALSRPGNKECNWYNKCPK | |
| | ECISGVYTDAYPLSPDAANVATVTLYANTSRVNPTIMYS | |
| | NTTNIINMLRIKDVQLEAAYTTTSCITHFGKGYCFHIIEIN | |
| | QKSLNTLQPMLFKTSIPKLCKAES | |
| 48 | MWSELKIRSNDGGEGPEDANDPRGKGVQHIHIQPSLPVG | Sendai HN protein |
| | QRVRMDGDRGKRDSYWSTSPSGSTTKPASGWERSSKA | (modified CTD) |
| | DTWLLILSFTQWALSIATVIICIIISARQGYSMKEYSMTVE | |
| | ALNMSSREVKESLTSLIRQEVIARAVNIQSSVQTGIPVLL | |
| | NKNSRDVIQMIDKSCSRQELTQHCESTIAVHHADGIAPL | |
| | EPHSFWRCPVGEPYLSSDPEISLLPGPSLLSGSTTISGCVR | |
| | LPSLSIGEAIYAYSSNLITQGCADIGKSYQVLQLGYISLNS | |
| | DMFPDLNPVVSHTYDINDNRKSCSVVATGTRGYQLCSM | |
| | PTVDERTDYSSDGIEDLVLDVLDLKGRTKSHRYRNSEV | |
| | DLDHPFSALYPSVGNGIATEGSLIFLGYGGLTTPLQGDTK | |
| | CRTQGCQQVSQDTCNEALKITWLGGKQVVSVIIQVNDY | |
| | LSERPKIRVTTIPITQNYLGAEGRLLKLGDRVYIYTRSSG | |
| | WHSQLQIGVLDVSHPLTINWTPHEALSRPGNKECNWYN | |
| | KCPKECISGVYTDAYPLSPDAANVATVTLYANTSRVNPT | |
| | IMYSNTTNIINMLRIKDVQLEAAYTTTSCITHFGKGYCFH | |
| | IIEINQKSLNTLQPMLFKTSIPKLCKAES | |

What is claimed:

1. A method of producing a lipid membrane bound particle, said method comprising culturing host cells comprising one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the pH of the medium during the culturing is maintained at a culture pH of 6.7 to 7.7,
wherein the lipid membrane bound particle produced by the method comprises a lipid bilayer and the one or more Paramyxovirus envelope protein or biologically active portion thereof are embedded in the lipid bilayer.

2. A method of producing a lipid membrane bound particle, said method comprising culturing host cells transfected with one or more nucleic acids for the production of a lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the pH of the medium during the culturing is maintained at a culture pH that is slightly basic,
wherein the lipid membrane bound particle produced by the method comprises a lipid bilayer enclosing a lumen and the one or more Paramyxovirus envelope protein or biologically active portion thereof are embedded in the lipid bilayer.

3. The method of claim 2, wherein at least one of the one or more nucleic acids for the production of the lipid membrane bound particle derived from a virus are stably expressed in the host cell, optionally wherein the one or more nucleic acids are integrated into the chromosome of the host cell.

4. The method of claim 1 or claim 2, wherein the nucleic acid encoding the one or more Paramyxovirus envelope protein or a biologically active portion thereof is introduced into the cell, optionally by transfection of the one or more nucleic acids.

5. A method of producing a lipid membrane bound particle, said method comprising culturing host cells transfected with one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the culturing the pH of the medium during the culturing is maintained at a culture pH of 6.7 to 7.7,
wherein the lipid membrane bound particle comprises a lipid bilayer and the one or more Paramyxovirus envelope protein or biologically active portion thereof are embedded in the lipid bilayer.

6. A method of producing a lipid membrane bound particle, said method comprising culturing host cells transfected with one or more nucleic acids for the production of the lipid membrane bound particle derived from a virus under conditions for producing the lipid membrane bound particle by the host cells, wherein one of the one or more nucleic acids further encodes one or more Paramyxovirus envelope protein or a biologically active portion thereof, and wherein the culturing the pH of the medium during the culturing is maintained at a culture that is slightly basic,
wherein the lipid membrane bound particle comprises a lipid bilayer and the one or more Paramyxovirus envelope protein or biologically active portion thereof are embedded in the lipid bilayer.

7. A method of culturing a cell, said method comprising culturing host cells comprising one or more nucleic acids for the production of the lipid membrane bound particle derived from a virus, wherein one of the one or more nucleic acids further encodes one or more Paramyxovirus envelope protein or a biologically active portion thereof, and wherein the pH of the medium during the culturing is maintained at a culture pH of 6.7 to 7.7.

8. A method of culturing a cell, said method comprising culturing host cost cells comprising one or more nucleic acids for the production of the lipid membrane bound particle derived from a virus, wherein one of the one or more nucleic acids further encodes one or more Paramyxovirus envelope protein or a biologically active portion thereof, and wherein the pH of the medium during the culturing is maintained at a culture pH that is slightly basic.

9. The method of claim 7 or claim 8, wherein the one or more nucleic acids for the production of the lipid membrane bound particle derived from a virus are stably expressed in the host cell, optionally wherein the one or more nucleic acids are integrated into the chromosome of the host cell.

10. The method of any of claims 7-9, wherein the nucleic acid encoding the one or more Paramyxovirus envelope protein or a biologically active portion thereof is introduced into the cell, optionally by transfection of the one or more nucleic acids.

11. The method of any of claims 1-4, 6, 8-10, wherein the pH of the medium during the culturing is maintained at a culture pH of 6.8 to 7.5.

12. The method of any of claims 1-4, 6, 8-10, wherein the pH of the medium during the culturing is maintained at a culture pH of 6.9 to 7.4.

13. The method of any of claims 1-12, wherein the pH of the medium during the culturing is maintained at a culture pH of 7 to 7.3.

14. The method of any one of claim 1-13, wherein the lipid membrane bound particle is a viral-like particle (VLP), or vector particle derived from a retrovirus.

15. The method of any one of claims 1-14, wherein the lipid membrane bound particle is a VLP or vector particle derived from a lentivirus.

16. A method of producing a lentiviral vector particle, said method comprising culturing host cells transfected with one or more nucleic acids for the production of a lentiviral lipid membrane bound particle, wherein one of the one or more nucleic acids further encodes one or more Paramyxovirus envelope protein or a biologically active portion thereof, and wherein the culturing is carried out in slightly basic medium under conditions for producing the lentiviral lipid membrane bound particle by the host cells.

17. The method of any one of claims 1-16, wherein the host cell is a mammalian cell, optionally wherein the host cell is selected from the group comprising HEK293 or 293T cells.

18. The method of claims 16 or 17, wherein the culturing is carried out in medium with a culture pH of 7.05-7.7.

19. The method of any one of claims 16-18, wherein the culturing is carried out in medium with a culture pH of 7.1-7.3.

20. The method of any of claims 1-19, wherein the culture pH is allowed to change at a pH set point with a deadband from 0.05, 0.10, 0.15, 0.20, 0.25, 0.30, 0.35, 0.40, 0.45 or 0.50 or any value between any of the foregoing.

21. The method of any of claims 1-20, wherein the culture pH is selected from: (i) pH 7.05±0.05; (ii) pH 7.15±0.05; (iii) pH 7.20±0.05; (iv) pH 7.35±0.05; (v) pH 7.05±0.10; (vi) pH 7.15±0.10; (vii) pH 7.20±0.10; (viii) pH 7.35±0.10; (ix) pH 7.05±0.15; (x) pH 7.15±0.15; (xi) pH 7.20±0.15; or (xii) pH 7.35±0.15.

22. The method of any of claims 1-21, wherein the culture pH is at or about 7.05, at or about 7.1, at or about 7.15, at or about 7.2, at or about 7.3, at or about 7.35.

23. The method of any of claims 1-22, wherein the culture pH is at or about 7.1.

24. The method of any of claims 1-22, wherein the culture pH is at or about 7.2.

25. The method of any of claims 1-22, wherein the culture pH is at or about 7.3.

26. The method of any of claims 1-25, wherein the medium has a dissolved oxygen concentration between 30 and 60 percent saturation.

27. The method of any of claim 1-26, wherein the medium has a dissolved oxygen concentration of 20, 30, 40, 50, or 60 percent saturation, or any value between any of the foregoing.

28. The method of any of claims 1-27, wherein the culturing is carried out in a bioreactor.

29. The method of claim 28, wherein the bioreactor is a stirred-tank bioreactor.

30. The method of any of claim 1-29, wherein the culturing is carried out in a volume of at least 1 L.

31. The method of any of claim 1-30, wherein the culturing is carried out in a volume of at least 5 L.

32. The method of any of claims 1-30, wherein the culturing is carried out in a volume between at or about 1 L-5 L, between at or about 5 L-10 L, between at or about 10 L-20 L, between at or about 20 L-50 L, between at or about 50-100 L, or between at or about 100-200 L.

33. The method of any of claims 1-28, wherein the method further comprises monitoring the pH of the medium and, optionally adjusting the pH to maintain the culture pH of the medium.

34. The method of any of claims 28-33, wherein the bioreactor comprises a pH adjustment module, wherein the pH adjustment module monitors the pH of the medium during the culturing.

35. The method of any of claims 28-34, wherein the bioreactor comprises a pH adjustment module, wherein the pH adjustment module maintains the pH of the medium during the culturing.

36. The method of claims 1-35, wherein the one or more Paramyxovirus envelope proteins have fusogenic activity.

37. The method of claims 1-36, wherein the native binding tropism of the one or more of the Paramyxovirus envelope proteins is reduced.

38. The method of claim 1-37, wherein the one or more Paramyxovirus envelope proteins is derived from an H protein molecule or a biologically active portion thereof from a Paramyxovirus and/or an HN protein molecule or a biologically active portion thereof from a Paramyxovirus.

39. The method of claim 1-38, wherein the one or more Paramyxovirus envelope proteins comprises an F protein molecule or a biologically active portion thereof and/or a glycoprotein G (G protein) or a biologically active portion thereof.

40. The method of claim 1-37, wherein the one or more Paramyxovirus envelope proteins comprises an F protein molecule or a biologically active portion thereof from a Paramyxovirus and a glycoprotein G (G protein) or a biologically active portion thereof from a Paramyxovirus.

41. The method of any of claims 1-40, wherein the paramyxovirus is a henipavirus.

42. The method of any of claims 1-41, wherein the paramyxovirus is Measles morbillivirus.

43. The method of any of claims 1-41, wherein the paramyxovirus is a Hendra virus.

44. The method of any of claims 1-41, wherein the paramyxovirus is Nipah virus.

45. The method of any of claims 39-41 and 44, wherein the F protein or the biologically active portion thereof is a wild-type Nipah virus F (NiV-F) protein or is a functionally active variant or biologically active portion thereof.

46. The method of any of claims 39-41, 44 or 45 wherein the F protein molecule or a biologically active portion thereof is a NiV-F protein that has the amino acid sequence set forth in SEQ ID NO: 4 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

47. The method of any of claims 39-41 and 44-46, wherein the NiV-F protein is a biologically active portion that is truncated and lacks up to 40 contiguous amino acid residues at or near the C-terminus of the wild-type NiV-F protein (SEQ ID NO:4).

48. The method of any of claims 39-41 and 44-47, wherein the NiV-F protein is a biologically active portion that is truncated at the C-terminus of wild-type NiV-F and has the sequence set forth in any of SEQ ID NOS: 5-15 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 5-15.

49. The method of any of claims 39-41 and 44-48, wherein the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:4.

50. The method of any of claims 39-41 and 44-49, wherein the F protein is a NiV-F protein that is a biologically active portion that has a 20 amino acid truncation at or near the C-terminus of the wild-type NiV-F protein (SEQ ID NO:4).

51. The method of claim 50, wherein the NiV-F protein has an amino acid sequence set forth in SEQ ID NO:37 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 37.

52. The method of any of claims 39-41 and 44-49, wherein the F protein is a NiV-F protein that is a biologically active portion thereof that has a 22 amino acid truncation at or near the C-terminus of the wild-type NiV-F protein (SEQ ID NO:4).

53. The method of claim 52, wherein the NiV-F protein has an amino acid sequence set forth in SEQ ID NO:32 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

54. The method of claim 52 or claim 53, wherein the NiV-F protein has an amino acid sequence set forth in SEQ ID NO:33 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 33.

55. The method of any of claims 38-41 and 44-54, wherein the F protein or the biologically active portion thereof is a NiV-F protein that is a functionally active variant that comprises a point mutation on an N-linked glycosylation site of the wild-type NiV-F protein (SEQ ID NO:4) or a biologically active potion thereof.

56. The method of any of claims 38-41 and 44-54, wherein the F protein or the biologically active portion thereof is a NiV-F protein that is a functionally active variant that comprises:
i) a 20 amino acid truncation at or near the C-terminus of the wild-type NiV-F protein (SEQ ID NO:4); and/or
ii) a point mutation on an N-linked glycosylation site.

57. The method of claim 56, wherein the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

58. The method of any of claims 39-41 and 43-57, wherein the G protein or the biologically active portion thereof is a wild-type Nipah virus G (NiV-G) protein or a Hendra virus G protein or is a functionally active variant or biologically active portion thereof.

59. The method of any of claims 39-41 and 44-58, wherein the G protein or the biologically active portion thereof is a wild-type NiV-G protein or a functionally active variant or biologically active portion thereof.

60. The method of any of claims 39-41 and 44-59, wherein the G protein or the biologically active portion thereof is a mutant NiV-G protein that is modified to exhibit reduced native binding tropism.

61. The method of any of claims 39-41 and 44-60, wherein the G protein or the biologically active portion thereof is a mutant NiV-G protein that exhibits reduced binding to Ephrin B2 or Ephrin B3.

62. The method of any of claims 39-41 and 44-61, wherein the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:1.

63. The method of any of claims 39-41 and 44-62, wherein the NiV-G protein is a biologically active portion that is truncated and lacks up to 40 contiguous amino acid residues at or near the N-terminus of the wild-type NiV-G protein (SEQ ID NO:1).

64. The method of any of claims 39-41 and 44-63, wherein the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:1, SEQ ID NO:2, or SEQ ID NO: 5.

65. The method of any of claims 39-41 and 44-64, wherein the NiV-G protein is a biologically active portion that is truncated at the N-terminus of wild-type NiV-G and has the sequence set forth in any of SEQ ID NOS: 2, 5, or 6 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs 1, 2, or 5.

66. The method of any of claims 39-41 and 44-65, wherein the G protein molecule or a biologically active portion thereof NiV-G protein has the amino acid sequence set forth in SEQ ID NO: 1 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1.

67. The method of any of claims 39-41 and 44-65, wherein the G protein or the biologically active portion thereof is a mutant NiV-G protein having the amino acid sequence set forth in SEQ ID NO: 2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:2.

68. The method of any of claims 39-41 and 44-65, wherein the G protein or the biologically active portion thereof is a mutant NiV-G protein having the amino acid sequence set forth in SEQ ID NO: 5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5.

69. The method of any of claims 39-41, 44-67, wherein the F protein comprises the sequence set forth in SEQ ID NO. 32 and the G protein comprises the sequence set forth in SEQ ID NO. 34.

70. The method of any of claims 1-69, wherein at least one of the one or more Paramyxovirus envelope proteins are linked to a secondary moiety that is a targeting domain or a functional domain.

71. The method of claim 70, wherein the at least one of the one or more Paramyxovirus is a glycoprotein G (G protein) or a biologically active portion thereof and the G protein or biologically active portion thereof is linked to the secondary moiety.

72. The method of claim 70 or claim 71, wherein the secondary moiety is a functional domain and the functional domain is selected from a cytokine, growth factor, hormone, neurotransmitter, receptor, or apoptosis ligand.

73. The method of claim 70 or claim 71, wherein the secondary moiety is a targeting domain and the targeting domain is specific for a cell surface receptor on a target cell.

74. The method of any of claim 70, wherein the targeting domain is a Design ankyrin repeat proteins (DARPin), a single domain antibody (sdAb), a single chain variable fragment (scFv), or an antigen-binding fibronectin type III (Fn3) scaffold.

75. The method of any one of claims 70-74, wherein the at least one of the one or more Paramyxovirus envelope proteins and the secondary moiety are directly linked.

76. The method of any one of claims 70-74, wherein the at least one of the one or more Paramyxovirus envelope proteins and secondary moiety are indirectly linked via a linker.

77. The method of claim 76, wherein the linker is a peptide linker.

78. The method of claim 77, wherein the peptide linker is (GₘS)ₙ(SEQ ID NO: 11), wherein each of m and n is an integer between 1 to 4, inclusive.

79. The method of any of claims 1-78, wherein one of the one or more nucleic acids for the production of the lipid membrane bound particle derived from a virus encodes an expression cassette comprising at least one retroviral gene, optionally wherein the at least one retroviral gene is a lentiviral gene.

80. The method of claim 79, wherein the at least one retroviral gene is a lentiviral gene selected from the group comprising gag, rev, and/or pol.

81. The method of claim 79 or claim 80, wherein the at least one retroviral gene is stably expressed in the host cell, optionally wherein the one or more nucleic acids are integrated into the chromosome of the host cell.

82. The method of any of claims 1-81, wherein one of the one or more nucleic acids for the production of the lipid membrane bound particle derived from a virus further encodes a transgene.

83. The method of any of claims 1-82, wherein one of the one or more nucleic acids for the production of the lipid membrane bound particle derived from a virus does not encode VSV-G.

84. The method of claim 15-83, wherein the lipid membrane bound particle derived from a lentivirus or the lentiviral lipid membrane bound particle is a VLP or vector particle derived from HIV-1.

85. The method of any of claims 15-83, wherein the lipid membrane bound particle derived from a lentivirus or the lentiviral lipid membrane bound particle is a VLP or vector particle derived from gamma-retrovirus (GaL-V).

86. The method of any of claims 1-6, 11-15, 23-85, wherein the method further comprises purifying the lipid membrane bound particle comprising the one or more Paramyxovirus envelope protein or biologically active portion thereof.

87. The method of any of claims 7-10, 16-19, wherein the method further comprises collecting the supernatant from the cell culture, said supernatant containing the lipid membrane bound particle produced by the host cells.

88. The method of claim 87, further comprising clarification and/or concentration of lipid membrane bound particles.

89. The method of any of claims 1-6, 11-15, 23-86, wherein the method further comprises purifying the lentiviral vector particles comprising the one or more Paramyxovirus envelope protein or biologically active portion thereof.

90. The method of claim 7-10, 16-19, 87-88, wherein the method further comprises collecting the supernatant from the cell culture, said supernatant containing the lentiviral vector particles produced by the host cells.

91. The method of claim 90, further comprising clarification and/or concentration of lentiviral vector particle.

92. The method of any of claims 88 or 91, wherein the clarification and/or concentration is by centrifugation.

93. The method of any of claims 88 or 91, wherein the clarification and/or concentration is by dialysis and/or filtration.

94. A composition comprising lipid membrane bound particles produced by the method of any of claims 1-93.

95. A composition comprising lentiviral vector particles produced by the method of any of claims 16-93.

96. The composition of any of claims 94-95, wherein the ratio of infectious titer to physical titer of the lipid membrane bound particles or lentiviral vector particles is 10,000:1, 5,000:1, 2,500:1, 1,000:1, 100:1 or lower.

97. The composition of any of claims 94-96, wherein the ratio of infectious titer to physical titer of the lipid membrane bound particles or lentiviral vector particles is between 10,000:1 and 100:1, 10,000:1 and 1,1000:1, 10:000:′ and 2,500:1, 1:10,000 and 5000:1, 5,000:1 and 100:1, 5,000:1 and 1000:1, 5000:1 and 2500:1, 2500:1 and 100:1, 2500:1 and 1000:1, or 1000:1 and 100:1.

98. The composition of any of claims 94-97, wherein the ratio of infectious titer to physical titer of the lipid membrane bound particles or lentiviral vector particles is between 10,000:1 and 1000:1.

99. The composition of any of claims 94-98, wherein the ratio of infectious titer to physical titer of the lipid membrane bound particles or lentiviral vector particles is between 10,000:1 and 5000:1.

100. A cell culture system, wherein said system comprises a vessel comprising cell culture medium and host cells, said vessel further comprising a pH monitoring module contacting cell culture medium comprising host cells transfected with one or more nucleic acids for the production of a lipid membrane bound particle derived from a virus, wherein at least one the one of the one or more nucleic acids further encodes an envelope protein derived from a Paramyxovirus.

101. The cell culture system of claim 100, wherein the lipid membrane bound particle is a retroviral vector particle, optionally a lentiviral vector particle.

102. A cell culture system, wherein said system comprises a vessel comprising cell culture medium and host cells, said vessel further comprising a pH monitoring module contacting the cell culture medium comprising host cells transfected with one or more nucleic acids for the production of a lentiviral vector particle, wherein the one or more nucleic acids further encodes an envelope protein derived from a Paramyxovirus.

103. A cell culture system, wherein said system comprises a vessel comprising at least 5 L of cell culture medium comprising host cells transfected with one or more nucleic acids for the production of a lipid membrane bound particle derived from a virus, wherein the at least one of the one or more nucleic acids further encodes an envelope protein derived from a Paramyxovirus.

104. The cell culture system of any of claims 100-103, wherein said vessel comprises a bioreactor.

105. The cell culture system of any of claims 100-104, wherein the cell culture system is for culturing the host cells under conditions for producing the lipid membrane bound particle by the host cells.

106. The cell culture system of any of claims 100-105, wherein the system further comprises a pH adjustment module, wherein the pH adjustment module monitors the pH of the medium during the culturing.

107. The cell culture system of any of claims 100-105, wherein the system further comprises a pH adjustment module, wherein the pH adjustment module maintains the pH of the medium during the culturing.

108. The cell culture system of claims 106 or 107, wherein the medium is maintained at a pH of 6.7 to 7.7.

109. The cell culture system of claims 106 or 107, wherein the medium is maintained at a pH of 7.1-7.3.

110. The cell culture system of any of claims 106-109, wherein the medium is maintained at a pH with a deadband from 0.05, 0.10, 0.15, 0.20, 0.25, 0.30, 0.35, 0.40, 0.45 or 0.50 or any value between any of the foregoing.

111. The cell culture system of any of claims 106-110, wherein the medium is maintained at a pH selected from: (i) pH 7.05±0.05; (ii) pH 7.15±0.05; (iii) pH 7.20±0.05;
(iv) pH 7.35±0.05; (v) pH 7.05±0.10; (vi) pH 7.15±0.10; (vii) pH 7.20±0.10; (viii) pH 7.35±0.10; (ix) pH 7.05±0.15; (x) pH 7.15±0.15; (xi) pH 7.20±0.15; or (xii) pH 7.35±0.15.

112. The cell culture system of any of claims 106-111, wherein the culture pH is at or about 7.05, at or about 7.1, at or about 7.15, at or about 7.2, at or about 7.3, at or about 7.35.

113. The cell culture system of claims 106-112, wherein the medium has a pH of at or about 7.1.

114. The cell culture system of claims 106-112, wherein the medium has a pH of at or about 7.2.

115. The cell culture system of claims 106-112, wherein the medium has a pH of at or about 7.3.

116. The cell culture system of claims 100-115, wherein the medium has a dissolved oxygen concentration between 40 and 60 percent saturation.

117. The cell culture system of claims 100-116, wherein the system further comprises a glucose monitor.

118. The cell culture system of claims 100-117, wherein the system further comprises a module for controlling stirring speed.

119. The cell culture system of claims 100-118, wherein the one or more Paramyxovirus envelope proteins have fusogenic activity.

120. The cell culture system of claims 100-119, wherein the native binding tropism of the one or more of the Paramyxovirus envelope proteins is reduced.

121. The cell culture system of claims 100-120, wherein the one or more Paramyxovirus envelope proteins is derived from an H protein molecule or a biologically active portion thereof from a Paramyxovirus and/or an HN protein molecule or a biologically active portion thereof from a Paramyxovirus.

122. The cell culture system of claims 100-120, wherein the one or more Paramyxovirus envelope proteins comprises an F protein molecule or a biologically active portion thereof and/or a glycoprotein G (G protein) or a biologically active portion thereof.

123. The cell culture system of claims 100-120, wherein the one or more Paramyxovirus envelope proteins comprises an F protein molecule or a biologically active portion thereof from a Paramyxovirus and a glycoprotein G (G protein) or a biologically active portion thereof from a Paramyxovirus.

124. The cell culture system of claims 100-123, wherein the paramyxovirus is a henipavirus.

125. The cell culture system of claims 100-123, wherein the paramyxovirus is Measles morbillivirus.

126. The cell culture system of claims 100-124, wherein the paramyxovirus is a Hendra virus.

127. The cell culture system of claims 100-124, wherein the paramyxovirus is Nipah virus.

128. The cell culture system of claims 100-124, 127, wherein the F protein or the biologically active portion thereof is a wild-type Nipah virus F (NiV-F) protein or is a functionally active variant or biologically active portion thereof.

129. The cell culture system of claims 100-124, 127, or 128, wherein the F protein molecule or a biologically active portion thereof is a NiV-F protein that has the amino acid sequence set forth in SEQ ID NO: 4 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

130. The cell culture system of claims 100-124, 127-129, wherein the NiV-F protein is a biologically active portion that is truncated and lacks up to 40 contiguous amino acid residues at or near the C-terminus of the wild-type NiV-F protein (SEQ ID NO:4).

131. The cell culture system of claims 100-124, 127-130, wherein the NiV-F protein is a biologically active portion that is truncated at the C-terminus of wild-type NiV-F and has the sequence set forth in any of SEQ ID NO. 32 or SEQ ID NO. 33 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO. 32 or SEQ ID NO. 33.

132. The cell culture system of claims 100-124, 127-131, wherein the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:4.

133. The cell culture system of claims 100-124, 127-132, wherein the F protein is a NiV-F protein that is a biologically active portion that has a 20 amino acid truncation at or near the C-terminus of the wild-type NiV-F protein (SEQ ID NO:4).

134. The cell culture system of claim 133, wherein the NiV-F protein has an amino acid sequence set forth in SEQ ID NO:37 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 37.

135. The cell culture system of claims 100-124, 127-132, wherein the F protein is a NiV-F protein that is a biologically active portion thereof that has a 22 amino acid truncation at or near the C-terminus of the wild-type NiV-F protein (SEQ ID NO:4).

136. The cell culture system of claim 135, wherein the NiV-F protein has an amino acid sequence set forth in SEQ ID NO:38 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 38.

137. The cell culture system of claim 135, wherein the NiV-F protein has an amino acid sequence set forth in SEQ ID NO:36 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36.

138. The cell culture system of any of claims 100-124, 127-137, wherein the F protein or the biologically active portion thereof is a NiV-F protein that is a functionally active variant that comprises a point mutation on an N-linked glycosylation site of the wild-type NiV-F protein (SEQ ID NO:4) or a biologically active potion thereof.

139. The cell culture system of any of claims 100-124, 127-137, wherein the F protein or the biologically active portion thereof is a NiV-F protein that is a functionally active variant that comprises:
i) a 20 amino acid truncation at or near the C-terminus of the wild-type NiV-F protein (SEQ ID NO:4); and/or
ii) a point mutation on an N-linked glycosylation site.

140. The cell culture system of claim 139, wherein the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

141. The cell culture system of any of claims 100-124, 127-140, wherein the G protein or the biologically active portion thereof is a wild-type Nipah virus G (NiV-G) protein or a Hendra virus G protein or is a functionally active variant or biologically active portion thereof.

142. The cell culture system of any of claims 100-124, 127-140, wherein the G protein or the biologically active portion thereof is a wild-type NiV-G protein or a functionally active variant or biologically active portion thereof.

143. The cell culture system of any of claims 100-124, 127-142, wherein the G protein or the biologically active portion thereof is a mutant NiV-G protein that is modified to exhibit reduced native binding tropism.

144. The cell culture system of any of claims 100-124, 127-143, wherein the G protein or the biologically active portion thereof is a mutant NiV-G protein that exhibits reduced binding to Ephrin B2 or Ephrin B3.

145. The cell culture system of any of claims 100-124, 127-144, wherein the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:1.

146. The cell culture system of any of claims 100-124, 127-145, wherein the NiV-G protein is a biologically active portion that is truncated and lacks up to 40 contiguous amino acid residues at or near the N-terminus of the wild-type NiV-G protein (SEQ ID NO:1).

147. The cell culture system of any of claims 100-124, 127-146, wherein the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:1, SEQ ID NO:2, or SEQ ID NO: 5.

148. The cell culture system of any of claims 100-124, 127-147, wherein the NiV-G protein is a biologically active portion that is truncated at the N-terminus of wild-type NiV-G and has the sequence set forth in any of SEQ ID NOS: 1, 2, or 5 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 1, 2, or 5.

149. The cell culture system of any of claims 100-124, 127-148, wherein the G protein molecule or a biologically active portion thereof NiV-G protein has the amino acid sequence set forth in SEQ ID NO: 1 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1.

150. The cell culture system of any of claims 100-124, 127-148, wherein the G protein or the biologically active portion thereof is a mutant NiV-G protein having the amino acid sequence set forth in SEQ ID NO: 2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:2.

151. The cell culture system of any of claims 100-124, 127-148, wherein the G protein or the biologically active portion thereof is a mutant NiV-G protein having the amino acid sequence set forth in SEQ ID NO: 5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5.

152. The cell culture system of any of claims 100-124, 122-124, 127-131, 135-148, 150 wherein the F protein comprises the sequence set forth in SEQ ID NO. 32 and the G protein comprises the sequence set forth in SEQ ID NO. 34.

153. The cell culture system of any of claims 100-124, wherein at least one of the one or more Paramyxovirus envelope proteins are linked to a secondary moiety that is a targeting domain or a functional domain.

154. The cell culture system of claim 153, wherein the at least one of the one or more Paramyxovirus is a glycoprotein G (G protein) or a biologically active portion thereof and the G protein or biologically active portion thereof is linked to the secondary moiety.

155. The cell culture system of claim 153 or claim 154, wherein the secondary moiety is a functional domain and the functional domain is selected from a cytokine, growth factor, hormone, neurotransmitter, receptor, or apoptosis ligand.

156. The cell culture system of claim 153 or claim 155, wherein the secondary moiety is a targeting domain and the targeting domain is specific for a cell surface receptor on a target cell.

157. The cell culture system of any of claim 153, wherein the targeting domain is a Design ankyrin repeat proteins (DARPin), a single domain antibody (sdAb), a single chain variable fragment (scFv), a VHH antibody (nanobody), or an antigen-binding fibronectin type III (Fn3) scaffold.

158. The cell culture system of any one of claims 153-157, wherein the at least one of the one or more Paramyxovirus envelope proteins and the secondary moiety are directly linked.

159. The cell culture system of any one of claims 153-157, wherein the at least one of the one or more Paramyxovirus envelope proteins and secondary moiety are indirectly linked via a linker.

160. The cell culture system of claim 159, wherein the linker is a peptide linker.

161. The cell culture system of claim 160, wherein the peptide linker is (GₘS)ₙ(SEQ ID NO: 11), wherein each of m and n is an integer between 1 to 4, inclusive.

162. A method of producing a lipid membrane bound particle, said method comprising culturing host cost cells in the cell culture system of any of claim 157, wherein said host cells comprise one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid bilayer.

163. A method of producing a lipid membrane bound particle, said method comprising culturing host cost cells in the cell culture system of any of claims 96-157, wherein said host cells are transfected with one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid bilayer.

164. A method of culturing a cell, said method comprising culturing host cells in the cell culture system of any of claims 100-161, wherein said host cells comprise one or more nucleic acids for the production of the lipid membrane bound particle derived from a virus, optionally wherein one of the one or more nucleic acids further encodes one or more Paramyxovirus envelope protein or a biologically active portion thereof, and wherein the culturing is carried out in medium with a culture pH of 6.7 to 7.7 under conditions for producing the lipid membrane bound particle by the host cells, wherein the lipid membrane bound particle comprises a lipid bilayer and the one or more Paramyxovirus envelope protein or biologically active portion thereof are embedded in the lipid.

165. A method of culturing a cell, said method comprising culturing host cells in the cell culture system of any of claims 100-161, wherein said host cells are transfected with one or more nucleic acids for the production of a lipid membrane bound particle derived from a virus, wherein one of the one or more nucleic acids further encodes one or more Paramyxovirus envelope protein or a biologically active portion thereof, and wherein the culturing is carried out in medium with a culture pH that is slightly basic under conditions for producing the lipid membrane bound particle by the host cells, wherein the lipid membrane bound particle comprises a lipid bilayer enclosing a lumen and the one or more Paramyxovirus envelope protein or biologically active portion thereof are embedded in the lipid bilayer.


SEQUENCE LISTING (48 sequences)

SEQ ID NO: 1 - protein, 602 aa, synthetic construct
  REGION 1..602: note = wild-type NiV-G protein
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 2 - protein, 601 aa, synthetic construct
  REGION 1..601: note = Nipah Virus G Protein (No Met)
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 3 - protein, 546 aa, synthetic construct
  REGION 1..546: note = Nipah virus F Protein
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 4 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 5 - protein, 602 aa, synthetic construct
  REGION 1..602: note = NiVG protein attachment glycoprotein (602 aa)
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 6 - protein, 604 aa, synthetic construct
  REGION 1..604: note = Hendra Virus G Protein
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 7 - protein, 603 aa, synthetic construct
  REGION 1..603: note = Hendra Virus G Protein without Met
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 8 - protein, 602 aa, synthetic construct; identical to SEQ ID NO: 1
  REGION 1..602: note = Nipah Virus G Protein

SEQ ID NO: 9 - protein, 601 aa, synthetic construct; identical to SEQ ID NO: 2
  REGION 1..601: note = Nipah Virus G Protein (No Met)

SEQ ID NO: 10 - protein, 622 aa, synthetic construct
  REGION 1..622: note = Cedar Virus G Protein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 11 - protein, 621 aa, synthetic construct
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 12 - protein, 632 aa, synthetic construct
  REGION 1..632: note = Bat Paramyxovirus G Protein
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 13 - protein, 631 aa, synthetic construct
  REGION 1..631: note = Bat Paramyxovirus G Protein (No Met)
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 14 - protein, 625 aa, synthetic construct
  REGION 1..625: note = Mojiang virus, Tongguan 1 G Protein
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 15 - protein, 624 aa, synthetic construct
  REGION 1..624: note = Mojiang virus, Tongguan 1 G (No Met)
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 16 - protein, 546 aa, synthetic construct
  REGION 1..546: note = Hendra virus F Protein
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 17 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 18 - protein, 546 aa, synthetic construct; identical to SEQ ID NO: 3
  REGION 1..546: note = Nipah virus F Protein

SEQ ID NO: 19 - protein, 520 aa, synthetic construct; identical to SEQ ID NO: 4
  REGION 1..520: note = Nipah virus F Protein, without signal sequence

SEQ ID NO: 20 - protein, 557 aa, synthetic construct
  REGION 1..557: note = Cedar Virus F Protein
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 21 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 22 - protein, 545 aa, synthetic construct
  REGION 1..545: note = Mojiang virus, Tongguan 1 F Protein
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 23 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 24 - protein, 662 aa, synthetic construct
  REGION 1..662: note = Bat Paramyxovirus F Protein
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 25 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 26 - protein, 26 aa, synthetic construct
  REGION 1..26: note = signal sequence
MVVILDKRCYCNLLILILMISECSVG

SEQ ID NO: 27 - protein, 83 aa, synthetic construct
  REGION 1..83: note = Nipah virus NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 28 - protein, 437 aa, synthetic construct
  REGION 1..437: note = Nipah virus NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 29 - protein, 597 aa, synthetic construct
  REGION 1..597: note = NiVG protein attachment glycoprotein Truncated 5
MKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 30 - protein, 582 aa, synthetic construct
  REGION 1..582: note = NiVG protein attachment glycoprotein Truncated 20
MSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 31 - protein, 577 aa, synthetic construct
  REGION 1..577: note = NiVG protein attachment glycoprotein Truncated 25
MSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 32 - protein, 498 aa, synthetic construct
  REGION 1..498: note = Truncated NiV fusion glycoprotein (FcDelta22) at cytoplasmic tail (without signal sequence)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 33 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 34 - protein, 569 aa, synthetic construct
  REGION 1..569: note = NiVG protein attachment glycoprotein Truncated and mutated (E501 A, W504A, Q530A, E533A) NiV G protein (Gc34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 35 - protein, 568 aa, synthetic construct
  REGION 1..568: note = NiVG protein attachment glycoprotein Truncated and mutated (E501 A, W504A, Q530A, E533A) NiV G protein (Gc34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 36 - protein, 524 aa, synthetic construct; identical to SEQ ID NO: 33
  REGION 1..524: note = Truncated NiV fusion glycoprotein (FcDelta22) at cytoplasmic tail (with signal sequence)

SEQ ID NO: 37 - protein, 500 aa, synthetic construct
  REGION 1..500: note = Nipah virus NiV-F F0 T234 truncation (aa 525-544)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 38 - protein, 498 aa, synthetic construct; identical to SEQ ID NO: 32
  REGION 1..498: note = Truncated mature NiV fusion glycoprotein (FcDelta22) at cytoplasmic tail

SEQ ID NO: 39 - protein, 552 aa, synthetic construct
  REGION 1..552: note = NivG protein attachment glycoprotein Without cytoplasmic tail Uniprot Q9IH62
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 40 - protein, 604 aa, synthetic construct; identical to SEQ ID NO: 6
  REGION 1..604: note = Hendra virus G protein Uniprot O89343

SEQ ID NO: 41 - protein, 603 aa, synthetic construct; identical to SEQ ID NO: 7
  REGION 1..603: note = Hendra virus G protein Uniprot O89343 Without N-terminal methionine

SEQ ID NO: 42 - protein, 555 aa, synthetic construct
  REGION 1..555: note = Hendra virus G protein Uniprot O89343 Without cytoplasmic tail
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 43 - skipped

SEQ ID NO: 44 - protein, 602 aa, synthetic construct; identical to SEQ ID NO: 5
  REGION 1..602: note = NiVG protein attachment glycoprotein (602 aa)

SEQ ID NO: 45 - skipped

SEQ ID NO: 46 - protein, 540 aa, synthetic construct
  REGION 1..540: note = Sendai F protein
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAEKR

SEQ ID NO: 47 - protein, 575 aa, synthetic construct
  REGION 1..575: note = Sendai HN protein
MDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 48 - protein, 618 aa, synthetic construct
  REGION 1..618: note = Sendai HN protein (modified CTD)
MWSELKIRSNDGGEGPEDANDPRGKGVQHIHIQPSLPVGQRVRMDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES